SEQUENCE LISTING (53 sequences)

SEQ ID NO: 1 - DNA, 333 bp, Homo sapiens
  source 1..333: mol_type = other DNA
gggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 2 - DNA, 217 bp, synthetic construct
  source 1..217: mol_type = other DNA
actaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 3 - DNA, 1017 bp, Homo sapiens
  source 1..1017: mol_type = other DNA
atggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaagggaagcctatcagactaacagcagatctctcggcagaaaccctacaagccagaagagagtgggggccaatattcaacattcttaaagaaaagaattttcaacccagaatttcatttccagccaaactaagcttcataagtgaaggagaaagaaaatactttacagacaagcaaatgctgagagattttgtcaccaccaggcctaccctaaaagagctcctgaaggaagcactaaacatggaaaggaacaaccggtaccagccgctgcaaaatcatgccaaaatgtaa

SEQ ID NO: 4 - DNA, 1104 bp, synthetic construct
  source 1..1104: mol_type = other DNA
ctagctagctagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaagggaagcctatcagactaacagcagatctctcggcagaaaccctacaagccagaagagagtgggggccaatattcaacattcttaaagaaaagaattttcaacccagaatttcatttccagccaaactaagcttcataagtgaaggagaaagaaaatactttacagacaagcaaatgctgagagattttgtcaccaccaggcctaccctaaaagagctcctgaaggaagcactaaacatggaaaggaacaaccggtaccagccgctgcaaaatcatgccaaaatggaacaaaaactcatctcagaagaggatctgaatatgcataccggtcatcatcaccatcaccattgactagctagctag

SEQ ID NO: 5 - DNA, 3828 bp, Homo sapiens
  source 1..3828: mol_type = other DNA
atgacaggatcaaattcacacataacaatattaactttaaatataaatggactaaattctgcaattaaaagacacagactggcaagttggataaagagtcaagacccatcagtgtgctgtattcaggaaacccatctcatgtgcagagacacacataggctcaaaataaaaggatggaggaagatctaccaagcaaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctgagtgacctacaaagagacttagactcccacacattaataatgggagactttaacaccccactgtcaatattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaatcgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaacaggaagaagttgaatctctgaatggaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccattgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagaggataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactaccttacttcaaactatactacaaggctacagtaaccaaaacagcatggtactggtaccaaaacagagatatagatcaatggaacagaacagagccctcagaaataatgccacatatctacaactatctgatctttgacaaacctgagaaaaacaagcaatggggaaaggattccctatttaataaatggtgctgggaaaactggctagccatatgtagaaagctgaaactggatctcttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccagaggactataaatcatgctgctataaagacacatgcactcgtatgtttattgcggcactattcacaatagcaaaaacttggaaccaacccaaatgtccaacaatgatagactggattaagaaaatgtggcacatatacaccatggaatattatgcagccataaaaaatgatgagttcatatcctttgtagggacatggatgaaattggaaaccatcattctcagtaaactatcgcaagaacaaaaaaccaaacaccgcatattctcactcataggtgggaattga

SEQ ID NO: 6 - DNA, 1115 bp, Mus musculus
  source 1..1115: mol_type = other DNA
atggcgaaaggtaaacggaggaatcttactaacaggaaccaagaccactcaccatcaccagaacccagcacacccacttcgcccagtccagggaaccccaacacacctgagaacctagacctagatttaaaagcatatctcatgatgatggtagagggcatcaagaaggactttaataaatcacttaaagaaatacaggagaacactgctaaagagttacaagtccttaaagaaaaacaggaaaacacaatcaaacaggtagaagtccttacagaaaaagaggaaaaaacatacaaacaggtgatggaaatgaacaaaaccatactagacctaaaaagggaagtagacacaataaagaaaactcaaagcgaggcaacactagagatagaaaccctaggaaagaaatctggaaccatagatttgagcatcagcaacagaatacaagagatggaagagagaatctcaggtgcagaacattccatagagaacatcggcacaacaatcaaagaaaatggaaaagcaaaaagatcctaactcaaaatatccaggaaatccaggacacaataagaagaccaaacgtacggataataggagtggatgagaatgaagattttcaactcaaaggtccagcaaacatcttcaacaaaattattgaagaaaacttcccaaatctaaagaatgagatgcatatgaacatacaagaagcctacagaactccaaatagactggaccagaaaagaaattcctcccgacacataataatcagaacatcaaatgcactaaataaagatagaatactaaaagcagtaagggaaaaaggtcaagtaacatataaaggcaagcctatcagaattacaccagatttttcaccagagactatgaaagccagaagagcctggacagatgttatacagacactaagagaacacaaactgcagcccaggctactatacccagccaaactctcaattatcatagagggagaaaccaaagtattccacgacaaaaccaaattcacgcattatctctccacgaatccagcccttcaaaggataataacagaaaaaaaccaatacaagaacgggaacaacgccctagaaaaaacaagaaggtaa

SEQ ID NO: 7 - DNA, 3846 bp, Mus musculus
  source 1..3846: mol_type = other DNA
atgccacctttaacaactaaaataacaggaagcaacaattacttttccttaatatctcttaacatcaatggtctcaactcgccaataaaaagacatagactaacaaactggctacacaaacaagacccaacattttgctgcttacaggaaactcatctcagagaaaaagatagacactacctcagaatgaaaggctggaaaacaattttccaagcaaatggtatgaagaaacaagcaggagtagccatcctaatatctgataagattgacttccaacccaaagtcatcaaaaaagacaaggagggacacttcattctcatcaaaggtaaaatcctccaagaggaactctcaattctgaatatctatgctccaaatacaagagcagccacattcactaaagaaactttagtaaagctcaaagcacacattgcgcctcacacaataatagtgggagacttcaacacaccactttcaccaatggacagatcatggaaacagaaactaaacagggacacactgaaactaacagaagtgatgaaacaaatggatctgacagatatctacagaacattttaccctaaaacaaaaggatataccttcttctcagcacctcatggtaccttctccaaaattgaccacataataggtcacaaatcaggcctcaacagattaaaaaatattgaaattgtcccatgtatcctatcagatcaccatgcactaaggctgatcttcaataacaaaataaataacagaaagccaacattcacatggaaactgaacaacactcttctcaatgataccttggtcaaggaaggaataaagaaagaaattaaagactttttagagtttaatgaaaatgaagccacaacgtacccaaacctttgggacacaatgaaagcatttctaagagggaaactcatagctatgagtgccttcaagaaaaaacgggagagagcacatactagcagcttgacaacacatctaaaagctctagaaaaaaaggaagcaaattcacccaagaggagtagacggcaggaaataatcaaactcaggggtgaaatcaaccaagtggaaacaagaagaactattcaaagaattaaccaaacgaggagttggttctttgagaaaatcaacaagatagataaacccttagctagactcactaaagggcacagggacaaaatcctaattaacaaaatcagaaatgaaaagggagacataacaacagatcctgaagaaatccaaaacaccatcagatccttctacaaaaggctatactcaacaaaactggaaaacctggacgaaatggacaaatttctggacagataccaggtaccaaagttgaatcaggatcaagttgaccttctaaacagtcccatatcccctaaagaaatagaagcagttattaatagtctcccagccaaaaaaagcccaggaccagacgggtttagtgcagagttctatcagaccttcaaagaagatctaactccagttctgcacaaactttttcacaagatagaagtagaaggtattctacccaactcattttatgaagccactattactctgatacctaaaccacagaaagatccaacaaagatagagaacttcagaccaatttctcttatgaacatcgatgcaaaaatccttaataaaattctcgctaaccgaatccaagaacacattaaagcaatcatccatcctgaccaagtaggttttattccagggatgcagggatggtttaatatacgaaaatccatcaatgtaatccattatataaacaaactcaaagacaaaaaccacatgatcatctcgttagatgcagaaaaagcatttgacaagatccaacacccattcatgataaaagttctggaaagatcaggaattcaaggccaatacctaaacatgataaaagcaatctacagcaaaccagtagccaacatcaaagtaaatggagagaagctggaagcaatcccactaaaatcagggactagacaaggctgcccactttctccctaccttttcaacatagtacttgaagtattagccagagcaattcgacaacaaaaggagatcaaggggatacaaattggaaaagaggaagtcaaaatatcactttttgcagatgatatgatagtatatataagtgaccctaaaaattccaacagagaactcctaaacctgataaacagcttcggtgaagtagctggatataaaattaactcaaacaagtcaatggcctttctctacacaaagaataaacaggctgagaaagaaattagggaaacaacacccttctcaatagccacaaataatataaaatatctcggcgtgactctaacgaaggaagtgaaagatctgtatgataaaaacttcaagtccctgaagaaagaaattaaagaagatctcagaagatggaaagatctcccatgctcatggattggcaggaccaacattgtaaaaatggctatcttgccaaaagcaatctacagattcaatgcaatccccattaaaattccaactcaattcttcaacgaattagaaggagcaatttgcaaattcatctggaataacaaaaaaccgaggatagcaaaaactcttctcaaggataaaagaacctctggtggaatcaccatgcctgacctaaagctttactacagagcaattgtgataaaaactgcatggtactggtatagagacagacaagtagaccaatggaatagaattgaagacccagaaatgaacccacacacctatggtcacttgatcttcgacaagggagccaaaaccatccagtggaagaaagacagcattttcaacaattggtgctggcacaactggttgttatcatgtagaagaatgcgaatcgatccatacttatctccttgtactaaggtcaaatctaagtggatcaaggaacttcacataaaaccagagacactgaaacttatagaggagaaagtggggaaaagtcttgaagatatgggcacaggggaaaaattcctgaacagaacagcaatggcttgtgctgtaagatcgagaattgacaaatgggacctaatgaaactccaaagtttctgcaaggcaaaagacactgtctataagacaaaaagaccaccaacagactgggaaaggatctttacctatcctaaatcagataggggactaatatccaacatatataaagaactcaagaaggtggacctcagaaaatcaaataacccccttaaaaaatggggctcagaactgaacaaagaattctcacctgaggaataccgaatggcagagaagcacctgaaaaaatgttcaacatccttaatcatcagggaaatgcaaatcaaaacaaccctgagattccacctcacaccagtgagaatggctaagatcaaaaattcaggtgacagcagatgctggcgaggatgtggagaaagaggaacactcctccattgttggtgggattgcaggcttgtacaaccactctggaaatcagtctggcggttcctcagaaaattggacatagtactaccggaggatccagcaatacctctcctgggcatatatccagaagaagccccaactggtaagaaggacacatgctccactatgttcatagcagccttatttataatagccagaaactggaaagaacccagatgcccctcaacagaggaatggatacagaaaatgtggtacatctacacaatggagtactactcagctattaaaaagaatgaatttatgaaattcctagccaaatggatggacctggagagcatcatcctgagtgaggtaacacaatcacaaaggaactcacacaatatgtactcactgataagtggatactag

SEQ ID NO: 8 - DNA, 405 bp, Homo sapiens
  source 1..405: mol_type = other DNA
gggtagagattcactgccttagtctcatgtagtctcgtgtagtcttttgagtaaataacataaagtatctcaagactttttcataacttgatattattttagtcttcctgaatttttaaatattgaaaagctgagtgtcttgtctgttttcctcccccttacactatagtgacggggctagtcaagctttggcaagttgccagagggacttccgcaacaaaccctatcctgtccgagcaaagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacg

SEQ ID NO: 9 - DNA, 150 bp, synthetic construct
  source 1..150: mol_type = other DNA
aggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggagg

SEQ ID NO: 10 - DNA, 555 bp, synthetic construct
  source 1..555: mol_type = other DNA
gggtagagattcactgccttagtctcatgtagtctcgtgtagtcttttgagtaaataacataaagtatctcaagactttttcataacttgatattattttagtcttcctgaatttttaaatattgaaaagctgagtgtcttgtctgttttaggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggaggcctcccccttacactatagtgacggggctagtcaagctttggcaagttgccagagggacttccgcaacaaaccctatcctgtccgagcaaagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacg

SEQ ID NO: 11 - DNA, 17 bp, Bacteriophage T7
  source 1..17: mol_type = other DNA
taatacgactcactata

SEQ ID NO: 12 - DNA, 789 bp, synthetic construct
  source 1..789: mol_type = other DNA
taatacgactcactatagggtagagattcactgccttagtctcatgtagtctcgtgtagtcttttgagtaaataacataaagtatctcaagactttttcataacttgatattattttagtcttcctgaatttttaaatattgaaaagctgagtgtcttgtctgttttaggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggaggcctcccccttacactatagtgacggggctagtcaagctttggcaagttgccagagggacttccgcaacaaaccctatcctgtccgagcaaagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacgactaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 13 - DNA, 572 bp, synthetic construct
  source 1..572: mol_type = other DNA
taatacgactcactatagggtagagattcactgccttagtctcatgtagtctcgtgtagtcttttgagtaaataacataaagtatctcaagactttttcataacttgatattattttagtcttcctgaatttttaaatattgaaaagctgagtgtcttgtctgttttaggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggaggcctcccccttacactatagtgacggggctagtcaagctttggcaagttgccagagggacttccgcaacaaaccctatcctgtccgagcaaagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacg

SEQ ID NO: 14 - DNA, 772 bp, synthetic construct
  source 1..772: mol_type = other DNA
gggtagagattcactgccttagtctcatgtagtctcgtgtagtcttttgagtaaataacataaagtatctcaagactttttcataacttgatattattttagtcttcctgaatttttaaatattgaaaagctgagtgtcttgtctgttttaggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggaggcctcccccttacactatagtgacggggctagtcaagctttggcaagttgccagagggacttccgcaacaaaccctatcctgtccgagcaaagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacgactaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 15 - DNA, 32 bp, synthetic construct
  source 1..32: mol_type = other DNA
ctatataagcagagctgggtagagattcactg

SEQ ID NO: 16 - DNA, 32 bp, synthetic construct
  source 1..32: mol_type = other DNA
ctctagttagccagaggatctccagcagttat

SEQ ID NO: 17 - DNA, 33 bp, synthetic construct
  source 1..33: mol_type = other DNA
aataactgctggagatcctctggctaactagag

SEQ ID NO: 18 - DNA, 32 bp, synthetic construct
  source 1..32: mol_type = other DNA
cagtgaatctctacccagctctgcttatatag

SEQ ID NO: 19 - DNA, 20 bp, synthetic construct
  source 1..20: mol_type = other DNA
cactgccacccagaagactg

SEQ ID NO: 20 - DNA, 20 bp, synthetic construct
  source 1..20: mol_type = other DNA
cctgcttcaccaccttcttg

SEQ ID NO: 21 - DNA, 20 bp, synthetic construct
  source 1..20: mol_type = other DNA
gacttatccatgtgcctgtt

SEQ ID NO: 22 - DNA, 18 bp, synthetic construct
  source 1..18: mol_type = other DNA
ttggctacgaaatgcttg

SEQ ID NO: 23 - DNA, 360 bp, Homo sapiens
  source 1..360: mol_type = other DNA
ggggttcggccctgcccgtagcacagccaagccctacctctcggttatcttttctcccgtcaccacccagtaaggtcatgtgcttccacccctggtcggatgtaacgctgccactcatgtcggtccctgagatccgggctgttgttgatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttg

SEQ ID NO: 24 - DNA, 150 bp, synthetic construct
  source 1..150: mol_type = other DNA
tgactactgagattactttgacatgtcccacttattaatatcaccttaagtttgggttcgattaatattatgtaacctgtgaacgagataagattctagagatttaatcgaaccttaattctgattcggttatgtcaaaaggtgtcttga

SEQ ID NO: 25 - DNA, 510 bp, synthetic construct
  source 1..510: mol_type = other DNA
ggggttcggccctgcccgtagcacagccaagccctacctctcggttatcttttctcccgtcaccacccagtaaggtcatgtgcttccacccctggtcggatgtaacgctgccactcatgtcggtccctgagatccgggctgttgttgtgactactgagattactttgacatgtcccacttattaatatcaccttaagtttgggttcgattaatattatgtaacctgtgaacgagataagattctagagatttaatcgaaccttaattctgattcggttatgtcaaaaggtgtcttgaatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttg

SEQ ID NO: 26 - DNA, 860 bp, synthetic construct
  source 1..860: mol_type = other DNA
taatacgactcactataggggttcggccctgcccgtagcacagccaagccctacctctcggttatcttttctcccgtcaccacccagtaaggtcatgtgcttccacccctggtcggatgtaacgctgccactcatgtcggtccctgagatccgggctgttgttgtgactactgagattactttgacatgtcccacttattaatatcaccttaagtttgggttcgattaatattatgtaacctgtgaacgagataagattctagagatttaatcgaaccttaattctgattcggttatgtcaaaaggtgtcttgaatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttggggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 27 - DNA, 527 bp, synthetic construct
  source 1..527: mol_type = other DNA
taatacgactcactataggggttcggccctgcccgtagcacagccaagccctacctctcggttatcttttctcccgtcaccacccagtaaggtcatgtgcttccacccctggtcggatgtaacgctgccactcatgtcggtccctgagatccgggctgttgttgtgactactgagattactttgacatgtcccacttattaatatcaccttaagtttgggttcgattaatattatgtaacctgtgaacgagataagattctagagatttaatcgaaccttaattctgattcggttatgtcaaaaggtgtcttgaatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttg

SEQ ID NO: 28 - DNA, 843 bp, synthetic construct
  source 1..843: mol_type = other DNA
ggggttcggccctgcccgtagcacagccaagccctacctctcggttatcttttctcccgtcaccacccagtaaggtcatgtgcttccacccctggtcggatgtaacgctgccactcatgtcggtccctgagatccgggctgttgttgtgactactgagattactttgacatgtcccacttattaatatcaccttaagtttgggttcgattaatattatgtaacctgtgaacgagataagattctagagatttaatcgaaccttaattctgattcggttatgtcaaaaggtgtcttgaatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttggggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 29 - DNA, 29 bp, synthetic construct
  source 1..29: mol_type = other DNA
ctatataagcagagctggggttcggccct

SEQ ID NO: 30 - DNA, 29 bp, synthetic construct
  source 1..29: mol_type = other DNA
agggccgaaccccagctctgcttatatag

SEQ ID NO: 31 - DNA, 18 bp, synthetic construct
  source 1..18: mol_type = other DNA
ccccagtacgatagcacc

SEQ ID NO: 32 - DNA, 20 bp, synthetic construct
  source 1..20: mol_type = other DNA
gacataaccgaatcagaatt

SEQ ID NO: 33 - DNA, 400 bp, Homo sapiens
  source 1..400: mol_type = other DNA
gagattcactgccttagtctcatgtagtctcgtgtagtcttttgagtaaataacataaagtatctcaagactttttcataacttgatattattttagtcttcctgaatttttaaatattgaaaagctgagtgtcttgtctgttttcctcccccttacactatagtgacggggctagtcaagctttggcaagttgccagagggacttccgcaacaaaccctatcctgtccgagcaaagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacg

SEQ ID NO: 34 - DNA, 550 bp, synthetic construct
  source 1..550: mol_type = other DNA
gagattcactgccttagtctcatgtagtctcgtgtagtcttttgagtaaataacataaagtatctcaagactttttcataacttgatattattttagtcttcctgaatttttaaatattgaaaagctgagtgtcttgtctgttttcctcccccttacactatagtgacggggctagtcaagctttggcaagttgccagagggacttccgcaacaaaccctatcctgtccgagcaaaggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggaggagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacg

SEQ ID NO: 35 - DNA, 17 bp, Bacteriophage SP6
  source 1..17: mol_type = other DNA
atttaggtgacactata

SEQ ID NO: 36 - DNA, 243 bp, synthetic construct
  source 1..243: mol_type = other DNA
acaatgagatcacatggacacaggaaggggaatatcacactctggggactgtggtggggtcgggggaggggggaggggtagcattgggagatatacctaatgctagatgacacattagtgggtgcagcgcaccagcatggcacatgtatacatatgtaactaacctgcacaatgtgcacatgtaccctaaaacttagagtataattaaaaaaaaaaaaaaaaaaaaaaaaaaaaaaaaaaaaa

SEQ ID NO: 37 - DNA, 810 bp, synthetic construct
  source 1..810: mol_type = other DNA
atttaggtgacactatagagattcactgccttagtctcatgtagtctcgtgtagtcttttgagtaaataacataaagtatctcaagactttttcataacttgatattattttagtcttcctgaatttttaaatattgaaaagctgagtgtcttgtctgttttcctcccccttacactatagtgacggggctagtcaagctttggcaagttgccagagggacttccgcaacaaaccctatcctgtccgagcaaaggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggaggagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacgacaatgagatcacatggacacaggaaggggaatatcacactctggggactgtggtggggtcgggggaggggggaggggtagcattgggagatatacctaatgctagatgacacattagtgggtgcagcgcaccagcatggcacatgtatacatatgtaactaacctgcacaatgtgcacatgtaccctaaaacttagagtataattaaaaaaaaaaaaaaaaaaaaaaaaaaaaaaaaaaaaa

SEQ ID NO: 38 - DNA, 213 bp, Homo sapiens
  source 1..213: mol_type = other DNA
atgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttg

SEQ ID NO: 39 - DNA, 103 bp, Homo sapiens
  source 1..103: mol_type = other DNA
aaaacaaaggtgccatgatgggctgttctaacccccacccccactgccaggtaagggtgtcaggggctccagtgggtttcttggctgagtctgagccagcact

SEQ ID NO: 40 - DNA, 30 bp, synthetic construct
  source 1..30: mol_type = other DNA
ctgaccatgcttatacggactatcgattag

SEQ ID NO: 41 - DNA, 660 bp, synthetic construct
  source 1..660: mol_type = other DNA
taatacgactcactataggggttcggccctgcccgtagcacagccaagccctacctctcggttatcttttctcccgtcaccacccagtaaggtcatgtgcttccacccctggtcggatgtaacgctgccactcatgtcggtccctgagatccgggctgttgttgtgactactgagattactttgacatgtcccacttattaatatcaccttaagtttgggttcgattaatattatgtaacctgtgaacgagataagattctagagatttaatcgaaccttaattctgattcggttatgtcaaaaggtgtcttgaatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttgctgaccatgcttatacggactatcgattagaaaacaaaggtgccatgatgggctgttctaacccccacccccactgccaggtaagggtgtcaggggctccagtgggtttcttggctgagtctgagccagcact

SEQ ID NO: 42 - DNA, 250 bp, Homo sapiens
  source 1..250: mol_type = other DNA
aaaatgccactgagaactctcttaagactacctttctccaaatggtgcccttcactcaagcctgtggttttggtcttaggaactttgctgccacaatacctcggcccttctcagttcgctacgacccatacacccaaaggattgaggtcttggacaatacccagcagcttaagattttggctgattccattaacagtaagtaatttacaccttacgaggccactcggtttctcagtaatcgaagactgtc

SEQ ID NO: 43 - DNA, 250 bp, Homo sapiens
  source 1..250: mol_type = other DNA
aaaatgccactgagaactctcttaagactacctttctccaaatggtgcccttcactcaagcctgtggttttggtcttaggaactttgctgccacaatacctcggcccttctcagttccctacgacccatacacccaaaggattgaggtcttggacaatacccagcagcttaagattttggctgattccattaacagtaagtaatttacaccttacgaggccactcggtttctcagtaatcgaagactgtc

SEQ ID NO: 44 - DNA, 700 bp, synthetic construct
  source 1..700: mol_type = other DNA
aaaatgccactgagaactctcttaagactacctttctccaaatggtgcccttcactcaagcctgtggttttggtcttaggaactttgctgccacaatacctcggcccttctcagttcgctacgacccatacacccaaaggattgaggtcttggacaatacccagcagcttaagattttggctgattccattaacagtaagtaatttacaccttacgaggccactcggtttctcagtaatcgaagactgtcaaaatgccactgagaactctcttaagactacctttctccaaatggtgcccttcactcaagcctgtggttttggtcttaggaactttgctgccacaatacctcggcccttctcagttccctacgacccatacacccaaaggattgaggtcttggacaatacccagcagcttaagattttggctgattccattaacagtaagtaatttacaccttacgaggccactcggtttctcagtaatcgaagactgtctttccctaccatcgccataggaaaaataataaatttattgaaatatttaattaaggagaaaagcacctccatgtaagccatgggttcattgatggagaagaacttgacaaaaaggtcagaattacccttgtgtcctttttcctttgaccttcctagattccactccacctcctaccatcattccacctttccacacttgg

SEQ ID NO: 45 - DNA, 917 bp, synthetic construct
  source 1..917: mol_type = other DNA
aaaatgccactgagaactctcttaagactacctttctccaaatggtgcccttcactcaagcctgtggttttggtcttaggaactttgctgccacaatacctcggcccttctcagttcgctacgacccatacacccaaaggattgaggtcttggacaatacccagcagcttaagattttggctgattccattaacagtaagtaatttacaccttacgaggccactcggtttctcagtaatcgaagactgtcaaaatgccactgagaactctcttaagactacctttctccaaatggtgcccttcactcaagcctgtggttttggtcttaggaactttgctgccacaatacctcggcccttctcagttccctacgacccatacacccaaaggattgaggtcttggacaatacccagcagcttaagattttggctgattccattaacagtaagtaatttacaccttacgaggccactcggtttctcagtaatcgaagactgtctttccctaccatcgccataggaaaaataataaatttattgaaatatttaattaaggagaaaagcacctccatgtaagccatgggttcattgatggagaagaacttgacaaaaaggtcagaattacccttgtgtcctttttcctttgaccttcctagattccactccacctcctaccatcattccacctttccacacttggactaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 46 - DNA, 32 bp, synthetic construct
  source 1..32: mol_type = other DNA
ctatataagcagagctaaaatgccactgagaa

SEQ ID NO: 47 - DNA, 33 bp, synthetic construct
  source 1..33: mol_type = other DNA
gttctcagtggcattttagctctgcttatatag

SEQ ID NO: 48 - DNA, 18 bp, synthetic construct
  source 1..18: mol_type = other DNA
agggaggtgtccgtgttc

SEQ ID NO: 49 - DNA, 18 bp, synthetic construct
  source 1..18: mol_type = other DNA
gggtgtatgggtcgtagc

SEQ ID NO: 50 - DNA, 350 bp, synthetic construct
  source 1..350: mol_type = other DNA
taatacgactcactatagggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtggtggcgggcgcctgtagtcccagctactcgggaggctgaggcaggagaatggcgtgaacccgggaggcggagcttgcagtgagccgagatcacgccgctgcactccaccctgggcgacagagcgagactccgtctcaaaaaaaaaaaaaaaaaaaaaaaaaaaaaagattaataactgctggagatc

SEQ ID NO: 51 - DNA, 335 bp, Homo sapiens
  source 1..335: mol_type = other DNA
ggccgggcgcggtggctcacgcctgtaatcccagcactttgggaggccgaggcgggtggatcatgaggtcaggagatcgagaccatcctggctaacaaggtgaaaccccgtctctactaaaaatacaaaaaaaaaaaattagccgggcgcggtggcgggcgcctgtagtcccagctactcgggaggctgaggcaggagaatggcgtgaacccgggaagcggagcttgcagtgagccgagattgcgccactgcagtccgcagtccggcctaggcgacagagcgagactccgtctcaaaaaaaaaaaaaaaaaaaatgtggccaaaagtgtcagaaa

SEQ ID NO: 52 - DNA, 217 bp, synthetic construct
  source 1..217: mol_type = other DNA
aaaaatacaaaaaaaaaaaattagccgggcgcggtggcgggcgcctgtagtcccagctactcgggaggctgaggcaggagaatggcgtgaacccgggaagcggagcttgcagtgagccgagattgcgccactgcagtccgcagtccggcctaggcgacagagcgagactccgtctcaaaaaaaaaaaaaaaaaaaatgtggccaaaagtgtcagaaa

SEQ ID NO: 53 - DNA, 789 bp, synthetic construct
  source 1..789: mol_type = other DNA
taatacgactcactatagggtagagattcactgccttagtctcatgtagtctcgtgtagtcttttgagtaaataacataaagtatctcaagactttttcataacttgatattattttagtcttcctgaatttttaaatattgaaaagctgagtgtcttgtctgttttaggtgcctgcacatactgcatgtgagagtctggagacgccagactgttctgagtcctgacctgctcaggggtgaggtccctctgagcctgagcaagcatttcgtagccaaccatgaatttccggacagtggcagagcgcaggagcggaggcctcccccttacactatagtgacggggctagtcaagctttggcaagttgccagagggacttccgcaacaaaccctatcctgtccgagcaaagattacctattaccagaacacactgacagtaagtaacatctatttagagagaatcaaataaacaatgttacagtatcacttttcattttgaatttttgatagaaattaaatgcacttaaatttggatatgcttacatactcttcattgttactctaagagaacgaaaaatacaaaaaaaaaaaattagccgggcgcggtggcgggcgcctgtagtcccagctactcgggaggctgaggcaggagaatggcgtgaacccgggaagcggagcttgcagtgagccgagattgcgccactgcagtccgcagtccggcctaggcgacagagcgagactccgtctcaaaaaaaaaaaaaaaaaaaatgtggccaaaagtgtcagaaa

(19) United States
(12) Patent Application Publication
(10) Pub. No.: US 2025/0197883 A1
(43) Pub. Date: Jun. 19, 2025

(54) RNA FRAMEWORK FOR GENE EDITING AND GENE EDITING METHOD

(71) Applicant: Yunpeng SUI, Beijing (CN)

(72) Inventors: Yunpeng SUI, Beijing (CN); Shuanghong PENG, Beijing (CN)

(73) Assignee: Yunpeng SUI, Beijing (CN)

(21) Appl. No.: 18/849,052

(22) PCT Filed: Dec. 23, 2022
(86) PCT No.: PCT/CN2022/141329

§ 371 Date: Sep. 20, 2024

(30) Foreign Application Priority Data
Mar. 21, 2022 (CN) 202210278164.5

(51) Int. Cl. C12N 15/85; A61K 31/713; C07K 14/435; C12N 15/90

(52) U.S. Cl. CPC C12N 15/85; A61K 31/713; C07K 14/435; C12N 15/902

(57) ABSTRACT

The present invention provides an RNA framework and a gene editing method for gene editing. The RNA framework comprises an upstream sequence of target site, a sequence to be inserted and a downstream sequence of target site along the direction of 5′→3′. Based on the intrinsic mechanism of eukaryotes, this gene editing method uses RNP or RNA (which can be prepared in vitro) and related proteins as vectors to transfer into the cytoplasm and nucleus to achieve gene editing of specific sequences or loci on the genome of the target system, such as insertion, deletion, replacement of specific sequences and site replacement, and has high targeting accuracy. The present invention is more suitable for further clinical application than other prior art because it does not introduce exogenous systems or substances such as proteins derived from prokaryotes and does not produce double-strand breaks.

### TECHNICAL FIELD

The present invention belongs to the technical field of biology, relates to a gene editing technology, and particularly relates to an RNA framework and a gene editing method for gene editing.

### BACKGROUND

The current gene editing technologies in the field of biology mainly include TALEN, ZFN, Targetron and CRISPR/Cas9 technologies. These technologies have been used so far and have been relatively mature, but still have obvious defects.

Among them, ZFN technology can only recognize sequences with a length of 9 bp, which makes its targeting accuracy poor. At the same time, this invention is complex, has a high off target rate, and high cytotoxicity, making it difficult to apply in practice. Although TALEN technology is simpler and has a longer recognition sequence than ZFN technology, the invention is still more complex, which hinders its further application in various fields. CRISPR/Cas9 technology is currently the mainstream gene editing technology, which is relatively easy to operate, but it still has an undeniable off target probability, and the risk of DNA double strand breaks caused by it hinders its further clinical application. The Targetron technology uses class II intron to introduce exogenous sequences into specific genomic loci, but this invention introduces class II intron into the genome, resulting in “scars”, and it only performs well in the field of bacterial gene editing, but is difficult to apply to other more advanced organisms.

These three technologies all have various obstacles to their application, such as off target, technical complexity, and unknown risks caused by double chain breakage. In addition, these three technologies will inevitably introduce genetic material and proteins that do not belong to the receiving system, leading to unpredictable effects and seriously hindering the clinical application of the above technologies.

### SUMMARY

To solve the above problems, the purpose of the present invention is to provide an RNA framework for gene editing. The RNA framework can realize the insertion, deletion, sequence replacement and site replacement of DNA in any region of a genome.

Another purpose of the present invention is to provide an RNP.

A third purpose of the present invention is to provide a DNA sequence.

A fourth purpose of the present invention is to provide a DNA vector.

A fifth purpose of the present invention is to provide a gene editing method.

A sixth purpose of the present invention is to provide an application of the RNA framework for gene editing.

To achieve the above purposes, the present invention provides an RNA framework for gene editing. The RNA framework comprises an upstream sequence of target site, a sequence to be inserted and a downstream sequence of target site along the direction of 5′→3′.
wherein the upstream sequence of target site on the RNA framework or a complementary sequence of the upstream sequence of target site is used for hybridizing with the upstream sequence of target site or the complementary sequence of the upstream sequence of target site for the target site in an eukaryotic genome or a prokaryotic genome;
the downstream sequence of target site or a complementary sequence of the downstream sequence of target site on the RNA framework is used for hybridizing with the downstream sequence of target site or the complementary sequence of the downstream sequence of target site for the target site in an eukaryotic genome or a prokaryotic genome;
the upstream sequence of target site and the downstream sequence of target site on the RNA framework are directly connected in the corresponding sequence in the genome; and the target site is between the upstream sequence of target site and the downstream sequence of target site in the sequence on the genome.

As mentioned above, the RNA framework for gene editing further comprising: one or more ORF2p functional initiation parts are directly or indirectly connected downstream of the downstream sequence of target site, or the downstream sequence of target site of the RNA framework for gene editing is replaced or partially replaced with one or more ORF2p functional initiation parts; wherein the multiple ORF2p functional initiation parts are directly or indirectly connected.

As mentioned above, one or more pan-ORF1p coding sequences and/or one or more pan-ORF2p coding sequences are further inserted into the ORF2p functional initiation part(s); wherein when one pan-ORF1p coding sequence or one pan-ORF2p coding sequence is inserted into the ORF2p functional initiation part(s), the ORF2p functional initiation part(s) are directly or indirectly connected with the pan-ORF1p coding sequence or the pan-ORF2p coding sequence; when a) multiple pan-ORF1p coding sequences, or b) multiple pan-ORF2p coding sequences, or c) the sum of the number of the pan-ORF1p coding sequence(s) and the pan-ORF2p coding sequence(s) is greater than or equal to two, pan-ORF1p coding sequence(s) and pan-ORF2p coding sequence(s) are inserted into the ORF2p functional initiation part(s), the pan-ORF1p coding sequences and the pan-ORF2p coding sequences are directly or indirectly connected, the pan-ORF1p coding sequences are directly or indirectly connected, the pan-ORF2p coding sequences are directly or indirectly connected, and the ORF2p functional initiation part(s) are directly or indirectly connected with the pan-ORF1p coding sequence(s) or the pan-ORF2p coding sequence(s).

As mentioned above, the RNA framework further comprising: one or more pan-ORF1p coding sequences and/or one or more pan-ORF2p coding sequences are directly or indirectly connected upstream of the upstream sequence of target site, and/or inside the upstream sequence of target site, and/or inside the downstream sequence of target site, and/or downstream of the downstream sequence of target site.

As mentioned above, when the one or more pan-ORF1p coding sequences and/or one or more pan-ORF2p coding sequences are located upstream of the upstream sequence of target site, inside the upstream sequence of target site, inside the downstream sequence of target site, and downstream of the downstream sequence of target site, and when a) the sum of the number of multiple pan-ORF1p coding sequences is greater than or equal to two, or b) the sum of the number of multiple pan-ORF2p coding sequences is greater than or equal to two, or c) the sum of the number of pan-ORF1p coding sequences and pan-ORF2p coding sequences is greater than or equal to two in the same position, the pan-ORF1p coding sequences and the pan-ORF2p coding sequences are directly or indirectly connected, the pan-ORF1p coding sequences are directly or indirectly connected and the pan-ORF2p coding sequences are directly or indirectly connected.

As mentioned above, the RNA framework for gene editing further comprising: one or more ORF2p functional initiation parts are directly or indirectly connected downstream of the downstream sequence of target site.

As mentioned above, when the one or more pan-ORF1p coding sequences and/or one or more pan-ORF2p coding sequences are located upstream of the upstream sequence of target site, inside the upstream sequence of target site and inside the downstream sequence of target site, and when a) the sum of the number of multiple pan-ORF1p coding sequences is greater than or equal to two, or b) the sum of the number of multiple pan-ORF2p coding sequences is greater than or equal to two, or c) the sum of the number of pan-ORF1p coding sequences and pan-ORF2p coding sequences is greater than or equal to two in the same position, the pan-ORF1p coding sequences and the pan-ORF2p coding sequences are directly or indirectly connected, the pan-ORF1p coding sequences are directly or indirectly connected and the pan-ORF2p coding sequences are directly or indirectly connected.

As mentioned above, when the one or more pan-ORF1p coding sequences and/or one or more pan-ORF2p coding sequences are located downstream of the downstream sequence of target site:
a) when there is one or more ORF2p functional initiation parts and one or more pan-ORF1p coding sequences, the one or more ORF2p functional initiation parts are located before or after the one or more pan-ORF1p coding sequences, or the ORF2p functional initiation parts are spaced apart from the pan-ORF1p coding sequences, the ORF2p functional initiation parts and the pan-ORF1p coding sequences are directly or indirectly connected, the multiple pan-ORF1p coding sequences are directly or indirectly connected, and the multiple ORF2p functional initiation parts are directly or indirectly connected; or
b) when there is one or more ORF2p functional initiation parts and one or more pan-ORF2p coding sequences, the one or more ORF2p functional initiation parts are located before or after the one or more pan-ORF2p coding sequences, or the ORF2p functional initiation parts are spaced apart from the pan-ORF2p coding sequences, the ORF2p functional initiation parts and the pan-ORF2p coding sequences are directly or indirectly connected, the multiple pan-ORF2p coding sequences are directly or indirectly connected, and the multiple ORF2p functional initiation parts are directly or indirectly connected; or
c) when there is one or more ORF2p functional initiation parts, one or more pan-ORF1p coding sequences and one or more pan-ORF2p coding sequences, the ORF2p functional initiation parts are located before or after the one or more pan-ORF1p coding sequences, or before or after the one or more pan-ORF2p coding sequences, or the one or more pan-ORF1p coding sequences are located before or after the one or more pan-ORF2p coding sequences, or the ORF2p functional initiation parts, the pan-ORF1p coding sequences and/or the pan-ORF2p coding sequences are arranged at intervals; the ORF2p functional initiation parts and the pan-ORF1p coding sequences are directly or indirectly connected, the ORF2p functional initiation parts and the pan-ORF2p coding sequences are directly or indirectly connected, and the multiple pan-ORF1p coding sequences are directly or indirectly connected; the multiple pan-ORF2p coding sequences are directly or indirectly connected, the multiple ORF2p functional initiation parts are directly or indirectly connected, and the pan-ORF1p coding sequences and the pan-ORF2p coding sequences are directly or indirectly connected.

As mentioned above, when one or more pan-ORF1p coding sequences and/or one or more pan-ORF2p coding sequences are further directly or indirectly connected into a single ORF2p functional initiation part of one or more ORF2p functional initiation parts in the RNA framework, and when one pan-ORF1p coding sequence or one pan-ORF2p coding sequence is inserted into the ORF2p functional initiation parts, the ORF2p functional initiation parts are directly or indirectly connected with the pan-ORF1p coding sequence or the pan-ORF2p coding sequence;
when a) multiple pan-ORF1p coding sequences, or b) multiple pan-ORF2p coding sequences, or c) the pan-ORF1p coding sequence(s) and the pan-ORF2p coding sequence(s), the sum of the number of the pan-ORF1p coding sequences and the pan-ORF2p coding sequences is greater than or equal to two, are inserted into the ORF2p functional initiation parts, the pan-ORF1p coding sequences and the pan-ORF2p coding sequences are directly or indirectly connected, the pan-ORF1p coding sequences are directly or indirectly connected, the pan-ORF2p coding sequences are directly or indirectly connected, and the ORF2p functional initiation parts are directly or indirectly connected with the pan-ORF1p coding sequences or the pan-ORF2p coding sequences.

As mentioned above, the RNA framework for gene editing wherein the downstream sequence of target site in the RNA framework is replaced or partially replaced with one or more ORF2p functional initiation parts; wherein when there are multiple ORF2p functional initiation parts, the ORF2p functional initiation parts are directly or indirectly connected.

As mentioned above, the sequence of the ORF2p functional initiation parts is a sequence of a short interspersed element RNA, a long interspersed element RNA, a short interspersed element derivative RNA, a long interspersed element derivative RNA, or a initiating ORF2p splicing and reverse transcription functional structure.

As mentioned above, the pan-ORF1p coding sequence is a modified sequence of the ORF1p coding sequence or the ORF1p coding sequence, and the pan-ORF2p coding sequence is a modified sequence of the ORF2p coding sequence or the ORF2p coding sequence.

As mentioned above, the RNA framework is obtained by prokaryotic system transcription, eukaryotic system transcription or chemical synthesis.

As mentioned above, the RNA framework is a linear RNA or located in a linear RNA, or a circRNA or located in a circRNA.

As mentioned above, the linear RNA in which the RNA framework is located or the circRNA in which the RNA framework is located is obtained by prokaryotic system transcription, eukaryotic system transcription or chemical synthesis.

The transcription process can occur in vitro or in a prokaryote or an eukaryote, in a tissue, an organ or a cell thereof.

As mentioned above, the prokaryotic transcription is transcription by an RNA polymerase of a prokaryote; and the eukaryotic transcription is transcription by an RNA polymerase I of an eukaryote, an RNA polymerase II of an eukaryote, or an RNA polymerase III of an eukaryote.

The present invention further provides an RNP, obtained by binding of the RNA framework for gene editing with ORF1p, ORF2p, ORF1p derivative proteins and/or ORF2p derivative proteins, or obtained by binding of the linear RNA in which the RNA framework for gene editing is located or the circRNA in which the RNA framework for gene editing is located with ORF1p, ORF2p, ORF1p derivative proteins and/or ORF2p derivative proteins.

The present invention further provides a DNA sequence which transcribes the above RNA framework for gene editing.

The present invention further provides a DNA sequence which transcribes the above linear RNA or the circRNA in which the RNA framework for gene editing is located.

As mentioned above, the DNA sequence wherein a prokaryotic promoter or an eukaryotic promoter is further directly or indirectly connected upstream of, downstream of and/or inside the DNA sequence.

As mentioned above, the prokaryotic promoter is T7, T3, T71ac, Sp6, araBAD, trp, lac, Ptac, pL, LacUV5, Tac, pBAD or pR.

As mentioned above, the eukaryotic promoter is CMV, pCMV, EF1a, SV40, human PGK1, mouse PGK1, Ubc, human beta actin, CAG, EFT3, TRE, UAS, Ac5, Polyhedrin, CaMKIIa, GAL1, GAL10, GAL1 and GAL10, GAL4, GAL80, TEF1, GDS, ADH1, CaMV35S, Ubi, H1, human U6 or mouse U6 promoter.

The present invention further provides a DNA vector which has the above DNA sequence.

The present invention provides a gene editing method, comprising the following steps:
1) selecting the target site to be edited in a genome, and determining an upstream sequence of target site and a downstream sequence of target site on both sides of the target site;
2) preparing the RNA framework for gene editing as mentioned above; and/or preparing the linear RNA or the circRNA as mentioned above in which the RNA framework for gene editing is located; and/or preparing the RNP as mentioned above; and/or preparing the DNA vector as mentioned above;
3a) transforming or transfecting the RNA framework into a cell, a tissue, an organ, or an organism to achieve gene editing;
or 3b) transforming or transfecting the linear RNA or the circRNA in which the RNA framework is located into a cell, a tissue, an organ or an organism to achieve gene editing;
or 3c) transforming or transfecting the RNP into a cell, a tissue, an organ or an organism to achieve gene editing;
or 3d) transforming or transfecting the DNA vector into a cell, a tissue, an organ or an organism to achieve gene editing;
or 3e) co-transforming or co-transfecting a variety of the RNA frame, the linear RNA or the circRNA in which the RNA framework is located, the RNP and/or the DNA vector into a cell, a tissue, an organ or an organism to achieve gene editing;
or 3f) co-transforming or co-transfecting one or more the RNA frame, the linear RNA or the circRNA in which the RNA framework is located, the RNP and the DNA vector, and ORF1p, ORF2p and ORF1p derivative proteins and/or ORF2p derivative proteins into a cell, a tissue, an organ or an organism to achieve gene editing.

The present invention further provides a gene editing method, comprising the following steps:
1) selecting the target site to be edited in a genome, and determining an upstream sequence of target site and a downstream sequence of target site on both sides of the target site;
2) preparing the RNA framework for gene editing as mentioned above; and/or preparing the linear RNA or the circRNA as mentioned above in which the RNA framework for gene editing is located; and/or preparing the RNP as mentioned above; and/or preparing the DNA vector as mentioned above;
3) preparing one or more auxiliary RNAs comprising ORF2p functional initiation part sequences, one or more pan-ORF1p coding sequences and/or one or more pan-ORF2p coding sequences, and/or one or more auxiliary RNPs obtained by binding the auxiliary RNAs with ORF1p, ORF2p, ORF1p derivative proteins and/or ORF2p derivative proteins, and/or one or more auxiliary DNA vectors that transcribe the ORF2p functional initiation parts, the pan-ORF1p coding sequences and/or the pan-ORF2p coding sequences;
4a) co-transforming or co-transfecting the RNA framework, and the auxiliary RNA, the auxiliary RNP and/or the auxiliary DNA vector prepared in step 3) into a cell, a tissue, an organ or an organism to achieve gene editing;
or 4b) co-transforming or co-transfecting the linear RNA or the circRNA in which the RNA framework for gene editing is located, and the auxiliary RNA, the auxiliary RNP and/or the auxiliary DNA vector prepared in step 3) into a cell, a tissue, an organ or an organism to achieve gene editing;
or 4c) co-transforming or co-transfecting the RNP, and the auxiliary RNA, the auxiliary RNP and/or the auxiliary DNA vector prepared in step 3) into a cell, a tissue, an organ or an organism to achieve gene editing;
or 4d) co-transforming or co-transfecting the DNA vector, and the auxiliary RNA, the auxiliary RNP and/or the auxiliary DNA vector prepared in step 3) into a cell, a tissue, an organ or an organism to achieve gene editing;
or 4e) co-transforming or co-transfecting a variety of the RNA framework, the linear RNA or the circRNA in which the RNA framework for gene editing is located, the RNP and/or the DNA vector, and the auxiliary RNA, the auxiliary RNP and/or the auxiliary DNA vector prepared in step 3) into a cell, a tissue, an organ or an organism to achieve gene editing;
or 4f) co-transforming or co-transfecting one or more the RNA framework, the linear RNA or the circRNA in which the RNA framework for gene editing is located, the RNP and/or the DNA vector, one or more the auxiliary RNA, the auxiliary RNP and/or the auxiliary DNA vector prepared in step 3), and one or more ORF1p, ORF2p, ORF1p derivative proteins and/or ORF2p derivative proteins into a cell, a tissue, an organ or an organism to achieve gene editing.

As mentioned above, the RNA framework, the linear RNA or the circRNA in which the RNA framework for gene editing is located, the RNP and the DNA vector transformed, transfected, co-transformed or co-transfected into the cell, the tissue, the organ or the organism is one or more; when there is one RNA framework or one linear RNA in which the RNA framework for gene editing is located or one circRNA in which the RNA framework for gene editing is located or one RNP or one DNA vector, a single place on the genome is edited;
when the sum of the RNA framework, the linear RNA or the circRNA in which the RNA framework for gene editing is located, the RNP and the DNA vector is greater than or equal to 2, and the upstream sequences of target site and/or the downstream sequences of target site in the RNA framework, the linear RNA or the circRNA in which the RNA framework for gene editing is located, the RNP and the DNA vector are different, multiple places on the genome are edited or operated.

The present invention provides an application of the above RNA framework for gene editing or the above linear RNA or the circRNA in which the RNA framework for gene editing is located or the above RNP or the above DNA vector as a drug for preventing and/or treating cancer, a gene-related disease or a neurodegenerative disease.

As mentioned above, the cancer is a glioma, a breast cancer, a cervical cancer, a lung cancer, a stomach cancer, a colorectal cancer, a duodenal cancer, a leukemia, a prostate cancer, an endometrial cancer, a thyroid cancer, a lymphoma, a pancreatic cancer, a liver cancer, a melanoma, a skin cancer, a pituitary tumor, a germinoma, a meningioma, a meningeal cancer, a glioblastoma, various astrocytomas, various oligodendrogliomas, an astrodendrocytomas, various ependymomas, a choroid plexus papilloma, a choroid plexus cancer, a chordoma, various gangliocytomas, a olfactory neuroblastoma, a sympathetic nervous system neuroblastoma, a pinealocytoma, a pineal blastoma, a medulloblastoma, a retina blastoma, a trigeminal schwannoma, a facial acoustic neuroma, a Glomus jugulare tumor, an angioreticuloma, a craniopharyngioma or a granular cell tumor.

As mentioned above, the gene-related disease is Huntington's disease, fragile X syndrome, phenylketonuria, pseudohypertrophic progressive muscular dystrophy, Duchenne muscular dystrophy, mitochondrial encephalomyopathy, mucopolysaccharidosis type I, mucopolysaccharidosis type II, mucopolysaccharidosis type IIIA, mucopolysaccharidosis type IIIB, mucopolysaccharidosis type IIIC, mucopolysaccharidosis type IIID, mucopolysaccharidosis type IVA, mucopolysaccharidosis type IVB, mucopolysaccharidosis type VI, mucopolysaccharidosis type VII, mucopolysaccharidosis type IX, spinal muscular atrophy, Parkinson's disease Syndrome, albinism, red green blindness, chondrodysplasia, enuresis, congenital deafness, thalassemia, sickle cell anemia, hemophilia, epilepsy related to genetic changes, myoclonus, dystonia, stroke and schizophrenia, vitamin D resistant rickets Familial colonic polyposis, 21 hydroxylase deficiency, arginase deficiency, Alport syndrome, Angelman syndrome, Reyna syndrome, atypical hemolytic uremia, autoimmune encephalitis, autoimmune pituitary inflammation, autoimmune insulin receptor disease, β-ketolytic enzyme deficiency, biotinidase deficiency, cardiac ion channel disease, primary carnitine deficiency, Castleman's disease, Charcot Marie Tooth disease, citrullinemia, congenital adrenal dysplasia, congenital hyperinsulinemia, congenital myasthenia gravis syndrome, non nutritive muscular rigidity syndrome, congenital scoliosis, coronary artery ectasia, congenital pure red blood cell aplasia Anemia Erdheim Chester's disease, Fabre's disease, familial Mediterranean fever, Fanconi anemia, galactosemia, Gaucher's disease, systemic myasthenia gravis, Gitelman syndrome, glutaratemia type I, glycogen storage disease (type I, type II), hemophilia, Wilson's disease, hereditary angioedema, hereditary epidermolysis bullosa, hereditary fructose intolerance, hereditary hypomagnesemia, hereditary multiple cerebral infarction dementia, hereditary spastic paraplegia, holocarboxylase synthase deficiency, homocysteinemia, homozygous familial hypercholesterolemia, HHH syndrome, hyperphenylalaninemia, hypoalkaline phosphatasia, hypophosphatemic rickets Diseases, idiopathic cardiomyopathy, idiopathic hypogonadotropic hypogonadism, idiopathic pulmonary hypertension, idiopathic pulmonary fibrosis IgG4 related diseases, congenital bile acid synthesis disorder, isovaleric acidemia, Kalman syndrome, Langerhans histiocytosis, Leren's syndrome, Leber hereditary optic neuropathy, long-chain 3-hydroxyacyl coenzyme A dehydrogenase deficiency, lymphangiomyomatosis, lysinuria protein intolerance, lysosomal acid lipase deficiency, maple syrup urine syndrome, Marfan syndrome, McCune Albright syndrome, medium chain acyl coenzyme A dehydrogenase deficiency, methylmalonic acidemia, multifocal motor neuropathy, multifocal acyl coenzyme A dehydrogenase deficiency, multiple sclerosis, Ankylosing muscular dystrophy, N-acetylglutamate synthase deficiency, neonatal diabetes, neuromyelitis optica, Niemann Pick disease, nonsyndromic deafness Noonan syndrome, ornithine aminotransferase deficiency, osteogenesis imperfecta, juvenile Parkinson's disease, early-onset Parkinson's disease, paroxysmal nocturnal hemoglobinuria, black spot polyp syndrome, POEMS syndrome, porphyria, Prader Willi syndrome, primary combined immunodeficiency, primary hereditary dystonia, primary light chain amyloidosis, progressive familial intrahepatic cholestasis, progressive muscular dystrophy, propionemia, alveolar proteinosis, pulmonary cystic fibrosis, retinitis pigmentosa, severe congenital neutropenia, severe infantile myoclonic epilepsy, Dravet syndrome Silver Russell syndrome, sitosterolemia, spinal medullary muscular atrophy, spinal muscular atrophy, spinocerebellar ataxia, systemic sclerosis, tetrahydrobiopterin deficiency, tuberous sclerosis, primary tyrosinemia, very long-chain acyl CoA dehydrogenase deficiency, Williams syndrome, eczema thrombocytopenia with immunodeficiency syndrome, X-linked agammaglobulinemia, X-linked adrenal leukodystrophy, X-linked lymphoproliferative disorder, arteriosclerotic cerebral small vessel disease, cerebral amyloid angiopathy, common cerebral artery disease with subcortical infarction and white matter encephalopathy, hidden cerebral artery disease with subcortical infarction and white matter encephalopathy, Cathepsin A-related arterial disease in stroke and white matter encephalopathy, pyridoxine dependent epilepsy, AADC enzyme deficiency in serotonin metabolism AADC deficiency or hereditary nephritis.

As mentioned above, the neurodegenerative disease is Parkinson's disease, Alzheimer's disease, Huntington's disease, amyotrophic lateral sclerosis, spinocerebellar ataxia, multiple system atrophy, primary lateral sclerosis, Pick's disease, frontotemporal dementia, Lewy body dementia, or progressive supranuclear palsy.

The present invention provides an application of the above RNA framework for gene editing or the above linear RNA or the circRNA in which the RNA framework for gene editing is located or the above RNP or the above DNA vector as a tool for insertion of target sequences, deletion of target sequences, replacement of target sequences, deletion of target sites, addition of target sites, addition of target sequences, replacement of target sites, inversion of target gene sequences, and/or inversion correction of target gene sequences.

The present invention provides an application of the above RNA framework for gene editing or the linear RNA or the circRNA in which the RNA framework for gene editing is located or the above RNP or the above DNA vector in production or amplification of a DNA template comprising the sequence of the above RNA framework.

The present invention provides an application of the above RNA framework for gene editing or the above linear RNA or the circRNA in which the RNA framework for gene editing is located or the above RNP or the above DNA vector or the above DNA template as a tool for increasing the gene editing efficiency of TALEN, ZFN, Targetron, Prime Editor, Twin Prime Editor, CRISPR or CRISPR/Cas9 technologies.

The present invention contains or can generate RNA, ssDNA, and/or dsDNA containing upstream sequence of target site, sequence to be inserted, downstream sequence of target site, and/or other sequences such as short interspersed elements, partial short interspersed elements, etc. These components can assist in homologous recombination or insertion of corresponding sequences into target sites using technologies such as TALEN, ZFN, Targetron, CRISPR, and CRISPR/Cas9, promoting the greater use of RNA, virus component-free transfection, and improving the genome sequence insertion efficiency of corresponding technologies (RNA transfer into cells does not require nuclear entry and can enter the nucleus during the non division phase under the binding and action of corresponding proteins such as ORF1p and/or ORF2p).

The downstream of the RNA framework of the present invention is connected with short interspersed element, partial short interspersed element, long interspersed element, partial long interspersed element, initiating ORF2p splicing and reverse transcription functional structure, allowing ORF2p encoded by long interspersed elements, especially human LINE-1, to bind to it, cut the single strand of target genome and use it as a primer for reverse transcription, ultimately forming dsDNA, and inserting the sequence to be inserted into the target site on the genome through homologous recombination. Due to the fact that the present invention only cuts a single strand of the genome and does not cause double strand breaks, it has high safety.

The present invention takes RNA and RNA binding with human endogenous protein(s) with specific functions (also known as RNP) as a functional body to implement a gene editing effect on the genome. The RNA is introduced into an organism for gene editing and is safer than DNA. At the same time, in vitro synthesis and transcription of RNA, especially prokaryotic in vitro transcription for production of RNA, make it easier to produce RNA in vitro, and convenient for further industrial production and commercialization.

In the present invention, ORF1p and/or ORF2p can bind to the RNA framework, protecting the RNA framework while assisting in transporting RNA into the nucleus; And the ORF2p expressed by the vector or in cell can only successfully slide from the 3′end of ssDNA produced by reverse transcription of the vector (RNA or/and RNP) to the cleavage site (target site) when the upstream sequence of target site on the vector matches the corresponding complementary sequence of the upstream sequence of target site on the genome, and further mediates the production of vector dsDNA, making the present invention highly accurate in targeting and greatly avoiding the widespread non-specific production of dsDNA in the nucleus that can have adverse effects on the genome. This theoretically makes its safety and accuracy higher than other existing gene editing technologies. At the same time, using RNA or RNP as vectors effectively solves the problem of difficult DNA entry into the nucleus, making it easy to perform gene editing on cells with low DNA transfection efficiency.

The present invention has the following beneficial effects:

The present invention provides an RNA framework for gene editing, which is based on the inherent mechanism of eukaryotes, using RNP or RNA (which can be prepared in vitro) and related proteins as vector, and transferring them into the cytoplasm and nucleus to achieve gene editing of specific sequence or site on the genome of target systems (such as cells, tissues, organs, or organisms), such as insertion, deletion, replacement of specific sequence, and site replacement, while maintaining high targeting accuracy. The present invention is more suitable for further practical applications such as clinical application compared to other existing technologies because it does not introduce exogenous systems or substances such as proteins derived from prokaryotes and does not produce double strand breaks. In addition, RNA can be obtained through in vivo or in vitro prokaryotic or eukaryotic promoter transcription or chemical synthesis, especially prokaryotic promoters have higher transcription efficiency, longer RNA product length, and can avoid splicing mechanisms in eukaryotic systems that damage the integrity of the RNA used. At the same time, protein ORF2p and/or ORF1p can also be synthesized in vitro for industrial mass production and commercialization.

### DESCRIPTION OF DRAWINGS

FIG. 1 is a schematic diagram of gene editing provided by the present invention.

FIG. 2 is a schematic diagram of an operation process in the present invention.

FIG. 3 is a basic structural schematic diagram of an RNA framework for gene editing provided by the present invention.

FIG. 4 is a schematic diagram of a 1^{st} improvement structure of an RNA framework for gene editing provided by the present invention.

FIG. 5 is a schematic diagram of a 2^{nd} improvement structure of an RNA framework for gene editing provided by the present invention.

FIG. 6 is a schematic diagram of a 3^{rd} improvement structure of an RNA framework for gene editing provided by the present invention.

FIG. 7 is a schematic diagram of a 4^{th} improvement structure of an RNA framework for gene editing provided by the present invention.

FIG. 8 is a schematic diagram of a 5^{th} improvement structure of an RNA framework for gene editing provided by the present invention.

FIG. 9 is a schematic diagram of a 6^{th} improvement structure of an RNA framework for gene editing provided by the present invention.

FIG. 10 is a schematic diagram of a 7^{th} improvement structure of an RNA framework for gene editing provided by the present invention.

FIG. 11 is a schematic diagram of a 8^{th} improvement structure of an RNA framework for gene editing provided by the present invention.

FIG. 12 is a schematic diagram of a 9^{th} improvement structure of an RNA framework for gene editing provided by the present invention.

FIG. 13 is a schematic diagram of a 10^{th} improvement structure of an RNA framework for gene editing provided by the present invention.

FIG. 14 is a schematic diagram of a 11^{th} improvement structure of an RNA framework for gene editing provided by the present invention.

FIG. 15 is a schematic diagram of a 12^{th} improvement structure of an RNA framework for gene editing provided by the present invention.

FIG. 16 is a schematic diagram of a 13^{th} improvement structure of an RNA framework for gene editing provided by the present invention.

FIG. 17 is a schematic diagram of a 14^{th} improvement structure of an RNA framework for gene editing provided by the present invention.

FIG. 18 is a schematic diagram of a 15^{th} improvement structure of an RNA framework for gene editing provided by the present invention.

FIG. 19 is a schematic diagram of a 16^{th} improvement structure of an RNA framework for gene editing provided by the present invention.

FIG. 20 is a schematic diagram of a 17^{th} improvement structure of an RNA framework for gene editing provided by the present invention.

FIG. 21 is a schematic diagram of a 18^{th} improvement structure of an RNA framework for gene editing provided by the present invention.

### DETAILED DESCRIPTION

Embodiments of the present invention will be described completely in detail below, to enable the advantages and features of the present invention to be understood more easily by those skilled in the art, so as to more clearly define the protection scope of the present invention.

The prior art such as CRISPR can cause double-strand break, is easy to introduce random sequences and mutation, and is low in efficiency of introducing target sequences.

The RNA framework for gene editing provided by the present invention is based on a transposon mechanism widely present in the eukaryote and a reconstruction mechanism mediated thereby for modifying components such as repetitive sequences and gene copies on the genome. This mechanism may cause deletion or addition of pathogenic trinucleotide repeat sequences in some central nervous system degenerative diseases such as Huntington's disease and fragile X syndrome, promote the amplification of HIV genomes in some proliferating immune cells on the human genome, and cause specific gene copy number increases and decreases in embryonic development and tumorigenesis.

The RNA framework and the corresponding RNP provided by the present invention do not cause double-strand break, carry out genome integration through homologous recombination, and are safer by contrast and convenient for practical application.

The RNA framework for gene editing provided by the present invention uses RNA or RNP as a vector (carrier), and accurately locates the sequence to be inserted into the selected gene site (target site) on the genome through the upstream sequence of the target site and the downstream sequence of the target site on both wings of the sequence to be inserted on the RNA or RNP (the upstream of the target site refers to the 5′ direction sequence of the target site on any single strand of the genome, and the downstream of the target site refers to the 3′ direction sequence of the target site on the corresponding single strand of the genome). At the same time, with the help of short interspersed element (SINE, short interspersed nuclear element) RNA, long interspersed element RNA (LINE, long interspersed nuclear element), short interspersed element derivative RNA, long interspersed element derivative RNA and/or the initiating ORF2p splicing and reverse transcription functional structure and proteins ORF2p (open reading frame 2 protein, L1 endonuclease) and/or ORF1p (open reading frame 1 protein) expressed by long interspersed elements, the sequence to be inserted is accurately inserted into the target site on the genome. ORF1p and/or ORF2p can be combined with RNA vectors, protecting RNA vectors while assisting in transporting RNA into the cell nucleus; and the ORF2p expressed in the vector or cell can only slide successfully from the 3′ end of the ssDNA formed by reverse transcription of the vector (RNA or/and RNP) to the shearing site (target site) when the upstream sequence of the target site on the vector is completely matched with the corresponding complementary sequence of the upstream sequence of the target site on the genome, shear the single strand on the genome and further mediate the formation of the vector dsDNA, so that the present invention has a high targeting accuracy, greatly avoiding the extensive and non-specific production of dsDNA that can have an adverse effect on the genome in the cell nucleus, which makes the safety and accuracy of the present invention theoretically higher than other existing gene editing technologies. At the same time, using RNA or RNP as a vector effectively solves the problem of DNA being difficult to enter the nucleus, and it is easy to perform gene editing on cells with low DNA transfection efficiency.

Short interspersed element RNA, long interspersed element RNA, short interspersed element derivative RNA, long interspersed element derivative RNA and/or functional structure for initiating ORF2p cleavage function and reverse transcription (initiating ORF2p splicing and reverse transcription functional structure) are connected downstream of the above RNA framework, so that ORF2p encoded by long interspersed elements (LINE), especially human LINE-1, binds thereto, cuts the single strand of the target genome and uses it as a primer for reverse transcription, finally forms dsDNA and inserts the sequence to be inserted into the target site on the genome through homologous recombination. Since the present invention only cuts the single strand of the genome and does not cause double-strand breaks in the genome, it has higher safety.

The present invention can produce RNA in eukaryotic or prokaryotic systems and cells, tissues, organs, and organisms through in vivo or in vitro expression, and produce the required proteins ORF1p and/or ORF2p in the target system or outside the target system (in vitro), and introduce the vector in the form of RNA or RNP into the target system such as cells, tissues, organs, and organisms to achieve the goal of gene editing, which is convenient for industrial mass production and commercialization.

In addition, since the splicing mechanism of the precursor mRNA in the eukaryotic system does not exist in the prokaryotic system or in vitro expression, the RNA framework and the downstream connectable short interspersed element RNA, short interspersed element derivative RNA, long interspersed element, long interspersed element derivative RNA, and/or the initiating ORF2p splicing and reverse transcription functional structure can be expressed unimpeded without suffering from potential splicing risks, thereby improving the production efficiency of the present invention and the effect of gene editing.

Therefore, the present invention can perform accurate sequence deletion, sequence replacement (substitution) and replacement (substitution) of individual sites etc. through homologous recombination or genome repair mechanism of the receiving system such as prokaryotes or eukaryotes itself on the basis of targeted insertion of the desired sequence into the genome. At the same time, based on the technical principles of the present invention, it can be known that the present invention can continue to design a vector and insert it through the new site formed after the previous sequence is inserted by the present invention. The progressive insertion makes the length of the inserted sequence theoretically unlimited, and can complete various types and forms of sequence insertion, deletion, replacement and site replacement and other gene editing purposes, and the use method is flexible. In addition, the present invention can also be used to perform gene editing on single or multiple CNV and its end to stabilize, extend, shorten or change its expression sequence, etc., thereby achieving the purpose of changing or stabilizing the gene expression and self-state of cells, tissues, organs, and organisms.

The sequence to be inserted in the RNA framework provided by the present invention can be an exogenous sequence or an endogenous sequence, and the length of the one-time inserted sequence is 1 bp-20000 bp. When inserted multiple times, the genome insertion of a DNA sequence of any length can be achieved. The length of the nucleotide sequence of the upstream sequence of the target site can be 1 bp-20000 bp, and the length of the nucleotide sequence of the downstream sequence of the target site can be 1 bp-20000 bp.

The present invention relates to short interspersed elements, long interspersed elements and related proteins generated therefrom, such as ORF1p, ORF2p, and other types of open reading frame proteins (ORFp). Short interspersed elements (SINEs) mainly include Alu elements (such as Alu Jo elements, Alu Jb elements, Alu Sq elements, Alu Sx elements, Alu Sp elements, Alu Sc elements, Alu Sg elements, Alu S elements, Alu Y elements, Alu Yb8 elements, Alu Ya5 elements, Alu Ya8 elements, Alu J elements, etc.) and SVA elements in primates (including humans), various mammalian-wide interspersed repeat elements (MIRs) commonly found in mammals, such as MIR and MIR3, etc., Mon-1 in monotremes, B1 and B2 elements in mice, C-element in rabbits, HE1 family in zebrafish, etc., SINE SmaI in salmon, Anolis SINE2 and Sauria SINE in reptiles, IdioSINE1, IdioSINE2, SepiaSINE, Sepioth-SINE1, Sepioth-SINE2A, Sepioth-SINE2B and OegopSINE in invertebrates such as squid, etc., and p-SINE1 in plants such as rice, etc. The long interspersed elements mainly include various LINE-1 (L1) in various different kinds of organisms, such as L1 in humans (L1RE1 (L1.2, LRE1) and LRE2), various LINE-2 (L2) and various LINE-3 (L3), Ta elements and six types of LINE: R2, RandI, L1, RTE, I and Jockey, as well as other LINE types such as LINE-1 in mice, LINE UnaL2 in eels, LINE R2 in insects, LINE ZfL2-1 and ZfL2-2 in zebrafish, L1 in algae, LINE SART1 in silkworms, L1 in monocots, Tad1 in fungi, L2 in fish and RTE in some mammals. In addition, LINE also includes L1s, L1spa, L1Or1, L1.2, F subfamily and TF subfamily, etc. in L1s, L1spa, L1Or1, L1.2, L1. SINE and LINE structures are widely present in various animals and plants and are scattered throughout the genome. Each organism has its own specific SINE and LINE corresponding to its function. The corresponding DNA sequence of Alu element is recorded as Alu sequence.

The main characteristics of SINEs are that they are relatively short transposons distributed on the genome, contain an internal RNA polymerase III promoter, and end with an A- or T-rich tail or a short simple repeat sequence, and are reverse transcribed with the help of LINEs. The right half of their transcription products contains a reverse transcription functional structure. LINEs are characterized by being widely distributed transposons on genome that contain reverse transcriptase coding sequence. SINEs and their corresponding LINEs in the corresponding species reconstruct the genome through similar mechanisms. The basic principle of this mechanism is to connect the lariat structure produced by processing pre-mRNA with the right half of SINE with reverse transcription functional structure produced by shearing of the transcription product of SINE. The RNA sequence of the right half of the SINE with the reverse transcription function structure left after the complete SINE transcription product is cut at the middle site is called partial short interspersed element RNA. To distinguish, its corresponding coding DNA sequence is called partial short interspersed element sequence. Different short interspersed element RNAs of different species have different cutting sites. The natural splicing site of short interspersed element RNA is generally located in the middle and front of the full length. For short interspersed element RNA with a total length of about 100-400 nt, its natural splicing site is usually located at the 100-250 nt. For example, for an Alu element with a total length of about 300 bp, the splicing site of its transcription product RNA (The scAlu splicing site or natural splicing site mentioned below is usually located before the middle poly A sequence of the Alu transcription product, but may fluctuate in actual situations) is located at the 118 nt. The spliced product contains Alu right monomer, and may also contain the middle poly A repeat sequence of the Alu element transcription product other than the Alu right monomer, or together with its upstream 2-3 bases, and the 3′ poly A repeat sequence behind the right monomer, which can be called partial Alu. For the sake of distinction, its corresponding coding DNA sequence is called a partial Alu sequence. For the transcription product RNA of various MIRs with a total length of about 260 nt, the splicing site can be observed within the range of 100-150 nt. In fact, no matter where the site is located, as long as the remaining right part of the transcription product contains a complete reverse transcription functional structure after splicing, it can function; the secondary structure of the reverse transcription functional structure forms a special structure, usually in the shape of Ω; Its primary structure is characterized by containing two sequences separated by an intermediate spacer sequence between the two sequences, and these two sequences can bind to the complementary sequence of the corresponding sequence on the genome that does not contain an intermediate spacer sequence and directly connects the two sequences; The ORF2p encoded by LINE can bind to the sequence located at the 3′ of the transcription product between the two sequences in the ORF2p functional initiation structure (part) in the above-mentioned transcription product, and cut the genome single strand at the genomic site corresponding to the gap between the two sequences to start reverse transcription. In addition, the corresponding transcription product (RNA sequence) of the Alu element is recorded as complete Alu.

A DNA sequence that contains a reverse transcription functional structure and can initiate reverse transcription but is different from conventional short interspersed elements in sequence (such as short interspersed elements with partial mutations but still with special structures and functions) is called a short interspersed element-like sequence (quasi-short interspersed element RNA) (SINE-similar), and its corresponding RNA sequence is called a short interspersed element-like RNA.

In addition, the RNA sequence containing the reverse transcription functional structure and the ORF2p binding sequence and the structure for initiating ORF2p endonuclease and reverse transcription is called “binding to ORF2p (For example, it has a poly A sequence, which is usually located on the right leg of the reverse transcription functional structure “Ω”) and initiating ORF2p splicing (cleavage) and reverse transcription functional structure”, which can be abbreviated as “initiating ORF2p splicing and reverse transcription functional structure”, and can also be called “ORF2p functional initiation structure (part)”; The transcription product of the “binding to ORF2p and initiating ORF2p splicing (cleavage) and reverse transcription functional structure” can form an “Ω” secondary structure due to its own factors or external factors; ORF2p can bind to the side of the “Ω” gap formed by the initiating ORF2p splicing and reverse transcription functional structure and is located at the 3′ position of the “Ω” structure. Through the proteins ORF1p and ORF2p expressed by the corresponding types of LINE, such as LINE-1, which functionally corresponds to the Alu element, and LINE-2, which corresponds to various MIR elements, RNA is converted into double-stranded DNA and binds to complementary, identical or similar sequences on the genome. The single-stranded DNA produced by reverse transcription of the RNA (transcription product) formed by transcription and the double-stranded DNA produced by the single-stranded DNA using the genomic sequence as a primer are the conversion products of the transcription product, and the insertion into the genome is completed by the homologous recombination mechanism after the formation of a specific “Ω” structure. In addition, LINE can also complete the above-mentioned similar conversion of RNA to double-stranded DNA and insertion into the genome by transcribing its downstream sequence (i.e., 3′ transduction) and binding to the complementary sequence on the genome to form an Ω structure. Take the Alu element and its corresponding LINE-1 that assists its function as an example: the pre-mRNA produced after gene expression can be cut to produce lariats with overlapping sequences. This can occur in any region of the pre-mRNA. The difference lies in the different strengths of the cutting that produces these lariats. Due to the generation of lariat upstream and downstream of exons (lariat sequence does not contain exons), its shearing strength based on sequence differences is higher than that of other surrounding lariat structures (such as lariat containing exons in the sequence), making it easy for exons to be completely cut out during pre-mRNA processing and inhibiting the generation of other lariats. At the same time, ORF1p produced by LINE-1 can protect the nucleic acid bound to it, and it and ORF2p, which is also produced by LINE, can locate the bound nucleic acid to the cell nucleus and transport it into the nucleus;

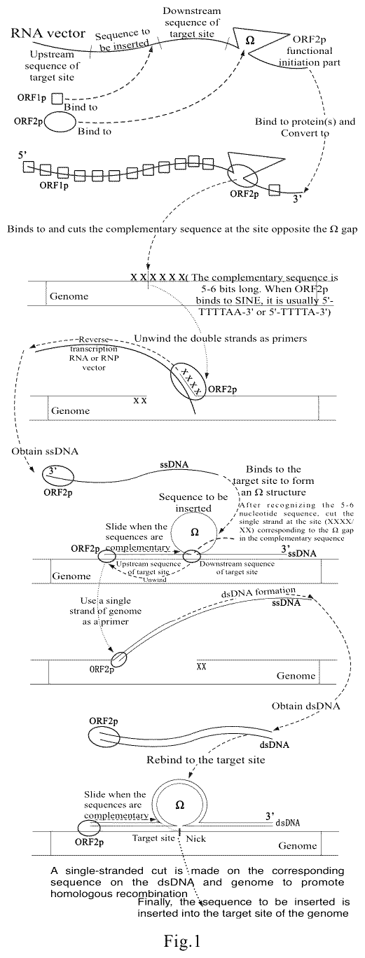

In addition, ORF2p can bind to the special Ω secondary structure of the Alu element transcription product and mediate the subsequent genome single-strand shearing, reverse transcription and auxiliary genome integration. As mentioned above, the transcription product of the Alu element can be sheared at a specific site to produce partial Alu. The lasso structure produced by shearing of the pre-mRNA can connect from its 3′ end to the remaining part of the Alu element transcription product containing the reverse transcription functional structure sheared (i.e., partial Alu). ORF2p can be recruited through ORF2p binding sequences, such as A-rich sequences, and bind to the 3′ foot of the two feet of the Ω structure formed by the partial Alu secondary structure, and recognize the sequence on the genome that matches the sequence on the two feet of Ω (mainly UU/AAAA, with discontinuity between U and A, i.e., the gap), cut the single strand of the genomic site opposite to the Ω gap and unwind the complementary sequence on the genome as a primer for reverse transcription. This process is called target-primed reverse transcription (TPRT); As reverse transcription proceeds, ORF2p moves to the 3′ end of the single-stranded DNA formed. The generated single-stranded DNA sequence can bind to the complementary sequence on the genome and form an Ω structure at the corresponding insertion site in the genome. Because the sequence to be inserted does not exist at the corresponding insertion site on the genome, and the sequences on both sides of the sequence to be inserted on the single-stranded DNA exist on both sides of the insertion site on the genome, ORF2p can slide to the Ω structure along the matching sequence in the 3′ to 5′ direction, recognize the 6-nucleotide sequence on the genome that is complementary to the sequence on both sides of the gap at the bottom of Ω, mainly the 4 nucleotides at 3′ and the 2 nucleotides at 5′, and form double-stranded DNA through a similar process as mentioned above. Note that only a completely matched sequence can allow ORF2p to slide to the cutting site, which ensures the accuracy of its targeting. The final double-stranded DNA is again in the shape of “Ω” and binds to the two sides of the corresponding insertion site on the genome that matches the sequences at both sides of the “Ω”. When ORF2p recognizes the middle discontinuity (Ω gap) of 6 nucleotides (mainly 4 nucleotides at 3′ and 2 nucleotides at 5′), it can create two single-stranded gaps in the genome corresponding to the gap and the other chain bound to itself through the endonuclease function of ORF2p, and insert the middle circular part into the genome with the help of homologous recombination mechanism. In this process, ORF1p can promote the formation of functional secondary and higher structures of the RNA used, and can promote the mutual binding and interaction between functional RNA and the genome interacting with it. By changing the inserted sequence, other effects such as deletion or replacement can be achieved through homologous recombination. In the above process, the annealing and deconstruction functions of ORF1p, which is also encoded by LINE, can also play an auxiliary role, helping to stabilize the secondary structure generated by nucleic acids in the above genome reconstruction process and their binding to the genome, as well as promoting the separation of nucleic acids from the genome after binding and interaction. In addition, ORF1p has a high RNA affinity and has a nuclear localization function. Since ORF2p can only cut one of the double-stranded chains of the genome and cannot produce double-strand breaks, it has a higher safety. Similar mechanisms also apply to other SINE and LINE combinations. Changes in local copy number variations in pathological and physiological processes such as embryonic development and tumorigenesis and the preference of the HIV-1 genome with deletions inserted into the human genome for short interspersed element sequences may be a manifestation of this mechanism in nature. It has been reported that the transcribed mRNA sequence can be integrated into the genome with the assistance of ORF1p and ORF2p, but because the transcription template is a pure exogenous non-homologous sequence, it cannot target a specific site in the genome, and the fragment with the reverse transcription functional structure is not connected, resulting in low efficiency and randomness, which is difficult to control. The present invention redesigns the transcription sequence and connects it with a sequence with a reverse transcription functional structure, such as various short interspersed element RNAs or partial short interspersed element RNAs, through various active or passive means to achieve a more accurate and efficient gene editing effect. In addition, ORF2p and ORF1p can also bind to long interspersed element RNA to mediate transposition activity. The 3′ part of the long interspersed element RNA that can form a special secondary structure and above structure can be intercepted, such as the corresponding transcription product (RNA sequence) of the 3′UTR part, which is called partial long interspersed element RNA. Partial long interspersed element RNA can be connected to the corresponding position such as the downstream of the RNA frame and function according to the above principle. The schematic diagram of the above basic principle is shown in FIG. 1.

According to the above principle, other sequences (such as sequences that are not homologous to the upstream sequence of the target site) should be avoided as much as possible upstream of the upstream sequence of the target site in the RNA framework. At the same time, the upstream sequence of the target site in the RNA framework should be as close as possible to or identical to the corresponding upstream sequence of the target site on the genome to improve gene editing efficiency.

In the present invention, the RNA sequence of the short interspersed element transcription product in nature (including natural mutations and other variations) is called short interspersed element RNA, and the RNA sequence of the long interspersed element transcription product in nature (including natural mutations and other variations) is called long interspersed element RNA. The short interspersed element derivative RNA or long interspersed element derivative RNA in the present invention refers to addition of other sequences, excision of part of the sequence, addition, deletion or sequence rearrangement of functional structure sequences, generation of similar sequences with similar functions, mixing of two element sequences, especially functional parts, etc. or a combination of the above changes on the basis of the short interspersed element RNA or the long interspersed element RNA. The short interspersed element derivative RNA or the long interspersed element derivative RNA has a similarity of not less than 50% with the short interspersed element RNA or the long interspersed element RNA in any continuous sequence of 10 bp or more. Short interspersed element derivative RNA includes partial short interspersed element RNA and other sequences that have changed based on the natural short interspersed element RNA sequence; long interspersed element derivative RNA includes partial long interspersed element RNA and other sequences that have changed based on the natural long interspersed element RNA sequence. In addition, 7SLRNA, which has a high similarity with short interspersed element RNA, also belongs to partial short interspersed element derivative RNA. The initiating ORF2p splicing and reverse transcription functional structure includes one or more of short interspersed element RNA, long interspersed element RNA, short interspersed element derivative RNA, long interspersed element derivative RNA and short interspersed element-like RNA.

The short interspersed element RNA, the long interspersed element RNA, the short interspersed element derivative RNA, the long interspersed element derivative RNA and/or the initiating ORF2p splicing and reverse transcription functional structure are collectively referred to as the ORF2p functional initiation part.

The ORF1p coding sequence in the present invention refers to the RNA sequence of the natural coding sequence of ORF1p in the long interspersed elements on the genome, and the ORF2p coding sequence refers to the RNA sequence of the natural coding sequence of ORF2p in the long interspersed elements on the genome. The modified sequence of the ORF1p coding sequence in the present invention can be modified from the ORF1p coding sequence, i.e., the natural ORF1p sequence, or the natural ORF1p sequence containing various variations or mutations. The modified sequence of the ORF2p coding sequence can be modified from the ORF2p coding sequence, i.e., the natural ORF2p sequence, or the natural ORF2p sequence containing various variations and mutations. The relevant modifications include additional addition of other sequences, truncation of partial sequences, addition, deletion or sequence rearrangement of functional structural sequences, generation of similar or similar sequences with similar or similar functions, mixing (fusion) of the entire or partial sequences of one or more other proteins (including ORF1p and ORF2p) with the entire or partial sequences of the ORF1p coding sequence and/or the ORF2p coding sequence, especially the functional sequences therein to form a corresponding fusion protein coding sequence, etc. or a combination of the above changes on the basis of the natural ORF1p coding sequence or the natural ORF2p coding sequence; The protein produced by the ORF1p coding sequence is called ORF1p, and the protein produced by the ORF2p coding sequence is called ORF2p; the protein produced by the modified sequence of the ORF1p coding sequence or the modified sequence of the ORF2p coding sequence is called ORF1p derivative protein or ORF2p derivative protein, respectively. The modified sequence of the ORF1p coding sequence or the modified sequence of the ORF2p coding sequence and the ORF1p derivative protein or ORF2p derivative protein expressed by them should still have the above functions and characteristics. The ORF1p coding sequence and the modified sequence of the ORF1p coding sequence are collectively referred to as the pan-ORF1p coding sequence, and the ORF2p coding sequence and the modified sequence of the ORF2p coding sequence are collectively referred to as the pan-ORF2p coding sequence.

ORF2p mainly contains several functional domains that are currently relatively clear, mainly the endonuclease domain (aa: 1-239), the cryptic region (aa: 240-347), the Z region (aa: 380-480), the reverse transcriptase domain (aa: 498-773), and the cysteine-rich domain (aa: 1130-1147).

Since the implementation of the present invention requires the endonuclease mechanism, adding an endonuclease domain, a portion of an endonuclease domain, or a modified structure having more than 50% amino acid homology with the endonuclease domain sequence in natural ORF2p to the ORF2p derivative protein can enhance the effect of the ORF2p derivative protein in the present invention;

The function of the cryptic region has been found to reduce the cytotoxicity of the endonuclease domain and increase the nuclear localization of the protein or polypeptide fragment in which it is located. In the present invention, more nuclear localization can increase the gene editing effect, and lower cytotoxicity is also beneficial to the practical application of the present invention. Therefore, adding or adding a longer cryptic region to the ORF2p derivative protein can promote the gene editing efficiency of the present invention to a certain extent;

It has been found that the cysteine-rich region can promote the binding of ORF2p derivative proteins to nucleic acids. Since the endonuclease action of ORF2p requires the assistance of special nucleic acids and their secondary structures to initiate, in the present invention, adding a cysteine-rich region, a part of the cysteine-rich region or a modified structure with more than 50% amino acid homology to the cysteine-rich region sequence in the natural ORF2p to the ORF2p derivative protein can improve the gene editing efficiency of ORF2p or is necessary;

The Z region can serve as a binding motif for PCNA, and can promote the role of ORF2p in the present invention. Therefore, in the present invention, adding the Z region, part of the Z region, or a modified structure having more than 50% amino acid homology with the Z region sequence in the natural ORF2p to the ORF2p derivative protein can improve the gene editing efficiency of the ORF2p derivative protein or is necessary.

In addition to the above-mentioned regions in natural ORF2p, the addition or partial addition of other regions in ORF2p derivative proteins, or the addition or partial addition of other regions of natural ORF2p that have been modified and maintain more than 50% homology with the original sequence to ORF2p derivative proteins can also improve the gene editing efficiency of the present invention to a certain extent.

The positional distribution of each region in the ORF2p derivative protein can be arranged according to the natural ORF2p or arranged in a new order or random order. Other regions can be added between or within each region. The amino acids in ORF2p and ORF2p derivative proteins can be replaced by corresponding conservative substitutions (Such as the mutual substitution between Phe, Trp, and Tyr, the mutual substitution between Leu, Ile, and Val, the mutual substitution between Gln and Asn, the mutual substitution between basic amino acids Lys, Arg, and His, the mutual substitution between acidic amino acids Asp and Glu, and the mutual substitution between hydroxyl amino acids Ser and Thr). In addition, the ORF2p derivative protein contains more homologous conservative amino acid sequences between human ORF2p and other species such as mouse ORF2p, which may improve the gene editing efficiency of the ORF2p derivative protein. The base sequence in the modified sequence of the ORF2p coding sequence encoding the ORF2p derivative protein can be replaced with different codon sequences for the same amino acid.

Current research has found that ORF1p mainly contains the following functional domains: N-terminal domain, coiled coil domain, RNA recognition motif, and C-terminal domain.

Since higher nucleic acid binding affinity and nucleic acid chaperone activity can improve the effect of the present invention, and the RNA recognition motif and C-terminal domain in ORF1p have the above functions in the protein, the ORF1p derivative protein contains the RNA recognition motif and/or C-terminal domain, part of the RNA recognition motif and/or C-terminal domain, or a modified structure having more than 30% amino acid homology with the RNA recognition motif or C-terminal domain sequence in natural ORF1p can improve the effect of the ORF1p derivative protein in the present invention;

The coiled-coil domain in ORF1p plays a role in the formation of trimers by ORF1p to improve nucleic acid binding affinity and promote transposition activity. Therefore, an ORF1p derivative protein containing a coiled-coil domain, a partial coiled-coil domain, or a modified structure having more than 30% amino acid homology with the coiled-coil domain in natural ORF1p can improve the role of the ORF1p derivative protein in the present invention;

The N-terminal domain also plays a role in the normal function of ORF1p, so the ORF1p derivative protein containing the N-terminal domain, part of the N-terminal domain or a modified structure having more than 30% amino acid homology with the N-terminal domain in the natural ORF1p can improve the function of the ORF1p derivative protein in the present invention;

Except for the above-mentioned regions in natural ORF1p, the addition or partial addition of other regions in ORF1p derivative proteins, or the addition or partial addition of other regions of natural ORF1p that have been modified and maintain more than 50% homology with the original sequence to ORF1p derivative proteins can improve the gene editing efficiency of the present invention to a certain extent.

Since protein phosphorylation plays a role in the normal function of ORF1p, adding a conserved proline-directed protein kinase (PDPK) site in ORF1p to the ORF1p derivative protein can improve the effect of the ORF1p derivative protein in the present invention.

The positional distribution of each region in the ORF1p derivative protein can be arranged according to the natural ORF1p or arranged in a new order or random order. Other regions can be added between or within each region. The amino acids in ORF1p and ORF1p derivative proteins can be replaced by corresponding conservative substitutions (Such as the mutual substitution between Phe, Trp, and Tyr, the mutual substitution between Leu, Ile, and Val, the mutual substitution between Gln and Asn, the mutual substitution between basic amino acids Lys, Arg, and His, the mutual substitution between acidic amino acids Asp and Glu, and the mutual substitution between hydroxyl amino acids Ser and Thr). In addition, the ORF1p derivative protein contains more homologous conservative amino acid sequences between human ORF1p and other species such as mouse ORF1p (For example, in the amino acid sequence of human ORF1p, ARR at positions 260-262, REKG at positions 235-238, and YPAKLS at positions 282-287 (Y at position 282 can be replaced by F with similar function)), which may improve the gene editing efficiency of the ORF1p derivative protein. The base sequence in the modified sequence of the ORF1p coding sequence encoding the ORF1p derivative protein can be replaced with different codon sequences for the same amino acid.

Sequences containing recombination sites (GCAGA[A/T]C, CCCA[C/G]GAC and/or CCAGC), short interspersed elements, partial short interspersed elements, short interspersed element derivatives, long interspersed elements (LINE), partial long interspersed elements and/or long interspersed element derivatives on the genome to be edited, or other sequences that can improve the efficiency of homologous recombination, can be searched for or (and) selected as corresponding sequences of the upstream and/or downstream sequences of the target site on the genome for sequence insertion, thereby improving the gene editing effect by increasing the efficiency of homologous recombination.

In the practical application of the present invention, if the RNA vector containing the RNA framework is not connected to the ORF2p function initiation structure downstream of the RNA framework and the expected efficiency is not achieved, or the connection efficiency of the RNA or its partial fragments with the short interspersed element RNA or its product is not high, increasing or decreasing the length of the upstream, downstream sequence of the target site and/or sequence to be inserted on the RNA framework to promote the connection can be tried; Alternatively, the lariat structure containing the site to be inserted produced in the corresponding prokaryotic or eukaryotic organism is detected according to the following detection method, and the sequence of the lariat structure is used as the upstream and/or downstream sequence of the target site or a part of the upstream and/or downstream sequence of the target site, or the upstream and/or downstream sequence of the target site on an RNA vector containing an RNA framework is appropriately extended, and the middle target site is the sequence to be inserted, to produce RNA; Alternatively, a poly A sequence can be added to the 3′ position of the RNA vector containing the RNA framework to promote the binding of ORF2p thereto. An ORF2p binding sequence such as a poly A sequence can be added to a suitable position on the RNA vector containing the RNA framework or an existing ORF2p binding sequence such as a poly A sequence can be extended without affecting the formation of the “Ω” structure of the RNA framework; The ORF2p binding sequence is mainly located at the 3′ part or 3′ end of the RNA vector containing the RNA framework, and the ORF2p binding sequence, such as the poly A sequence, can be added in, between, before, or after, each protein expression sequence (such as the pan-ORF1p coding sequence or the pan-ORF2p coding sequence), the upstream sequence of the target site (target site upstream sequence) in the RNA framework, the downstream sequence of the target site (target site downstream sequence) in the RNA framework, the short interspersed element RNA, the long interspersed element RNA, the short interspersed element derivative RNA, the long interspersed element derivative RNA and/or the initiating ORF2p splicing and reverse transcription functional structure or other sequences on the RNA on the RNA vector containing the RNA framework, or extend its inherent ORF2p binding sequence, such as the poly A sequence to improve the gene editing efficiency; Alternatively, a sequence may be designed at the 3′ position of an RNA vector containing an RNA framework to generate an “Ω” structure to promote ORF2p endonuclease.

In addition, since the gene editing effect of the present invention involves a homologous recombination mechanism, the co-application of a recombinase such as a site-specific serine recombinase with the present invention may increase the efficiency and effect of the present invention.

In addition, the target region (target site) for gene editing in the present invention may be one or more sites. When the inserted sequences for gene editing at two or more target sites have partially or completely the same or similar sequences (with a high degree of similarity) and the length of the partial sequence is 20 bp or more, the region between the two or more target sites for gene editing may be deleted or replaced with the inserted sequence or partial sequence.

When the sequence to be inserted in the present invention is short (100 bp or less), the sequence to be inserted may be inserted into the target site on the genome through homologous recombination and/or other genome repair mechanisms, thereby having a higher genome insertion efficiency.

When the upstream sequence of the target site and/or the downstream sequence of the target site in the present invention is short (100 bp or less), the sequence to be inserted may be inserted into the target site on the genome through homologous recombination and/or other genome repair mechanisms, thereby having a higher genome insertion efficiency.

It should be noted that the site to be inserted described in the present invention is a target site.

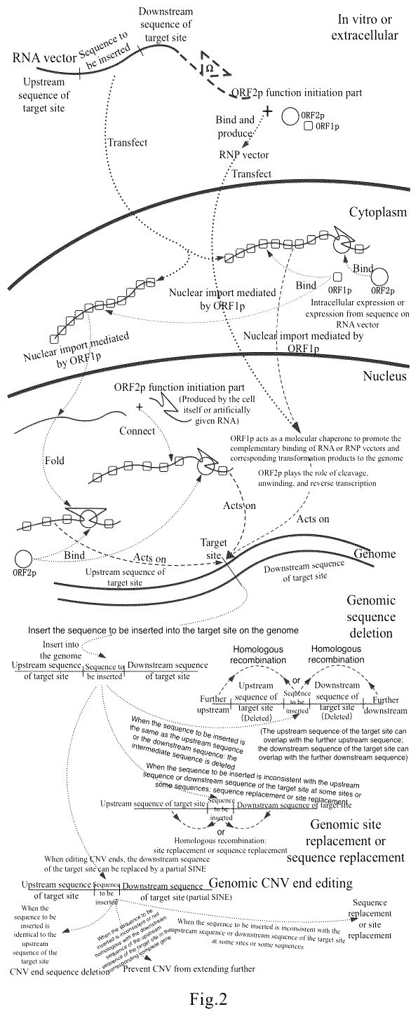

FIG. 2 shows a schematic diagram of an operation process in the present invention. The gene editing technology can realize “genome sequence insertion technology using RNA as a vector”, “genome sequence insertion technology using RNP as a vector”, “RNA vector and/or RNP vector-mediated genome sequence deletion technology”, “genome sequence replacement technology (including sequence replacement, site deletion, site addition, sequence addition, sequence deletion and site replacement)”, “technologies that block genome changes caused by transposons and stabilize the genomes and CNVs thereof”, “assisting other gene editing technologies”, etc., which will be described one by one below.

### I. Genome Sequence Insertion Technology Using RNA as a Vector

(1) Insertion of sequences into genome using RNA as a vector and mediated by a simple RNA framework: Select the upstream and downstream sequences (sequences upstream of the target site and sequences downstream of the target site) of the site to be inserted (i.e., the target site), add the sequence to be inserted at the insertion point (insertion site) between the upstream and downstream sequences, and use the designed sequence to generate RNA as a vector for production. The RNA can be placed in a solution containing an RNase inhibitor and/or an appropriate amount of Mg2+ (such as 6 mmol/L) or other metal ions or in a cell solution to promote the correct folding of the RNA and promote the subsequent binding with corresponding functional proteins such as ORF2p and/or ORF1p. Thereafter, the vector is transferred into cells cultured in vitro, tissues, organs, or administered to tissues, organs or organisms via pathways such as blood, lymph and cerebrospinal fluid, or local tissue administration through conventional means such as liposome transfection, so that the vector enters the cytoplasm of the target cell and binds to ORF1p and/or ORF2p before entering the nucleus, or ORF1p and/or ORF2p can mediate the vector to directly enter the nucleus. The vector RNA is connected to the short interspersed element RNA or its product transcribed in the cell and then binds to ORF2p expressed in the cell or binds to ORF2p and ORF1p at the same time; or directly binds to ORF2p expressed in the cell or binds to ORF2p and ORF1p at the same time (for example, when the Ω structure formed by the upstream sequence of the target site, the downstream sequence of the target site and the intermediate sequence to be inserted in the vector combines with the genome to replace the reverse transcription functional structure in the short interspersed element or its product to initiate reverse transcription), and the sequence to be inserted is inserted into the corresponding target site (site to be inserted) on the genome.

If the above method is continued according to the new site generated after insertion, the insertion can be continuous and the long fragment insertion without obvious length limit can be completed. If directional transfer is required, the outer package of the vector can be modified. Pay attention to avoid RNA degradation during the whole process.

Sequences containing recombination sites (GCAGA[A/T]C, CCCA[C/G]GAC and/or CCAGC), short interspersed elements, partial short interspersed elements, short interspersed element derivatives, long interspersed elements (LINE), partial long interspersed elements and/or long interspersed element derivatives on the genome to be edited, or other sequences that can improve the efficiency of homologous recombination, can be searched for or (and) selected as corresponding sequences of the upstream and/or downstream sequences of the target site on the genome for sequence insertion, thereby improving the gene editing effect by increasing the efficiency of homologous recombination.

Increasing the recombination sites (GCAGA[A/T]C, CCCA[C/G]GAC and/or CCAGC), short interspersed elements, partial short interspersed elements and/or short interspersed element derivative sequences in the corresponding sequences on the genome of the upstream and/or downstream sequences of the target site may increase the corresponding gene editing effects.

(2) Insertion of sequences into genome mediated by RNA as a vector and an RNA framework with one or more ORF2p functional initiation structure(s) (part(s)) connected downstream (in order to minimize the impact on the receiving system, the types of short interspersed element RNA, short interspersed element derivative RNA, long interspersed element RNA, long interspersed element derivative RNA, ORF1p and/or ORF2p in the corresponding receiving system can be selected):

This method does not require that the product of short interspersed element RNA after cutting be connected to the lariat formed by the vector RNA in vivo, but directly connects the RNA framework consisting of the upstream and/or downstream sequences of the insertion site (target site) and the intermediate sequence to be inserted (The upstream and downstream sequences of the site to be inserted (i.e. the target site) (the upstream sequence of the target site and the downstream sequence of the target site) (within 20,000 bp respectively), add the sequence to be inserted at the insertion point between the upstream and downstream sequences (within 20,000 bp)) to one or more ORF2p functional initiation part(s) and/or one or more other related sequence(s) downstream, and produces the RNA generated by the designed sequence as a vector. The RNA can be placed in a solution containing an RNase inhibitor and/or an appropriate amount of Mg2+ (such as 6 mmol/L) or other metal ions or in a cell solution to promote the correct folding of the RNA and promote the subsequent binding with corresponding functional proteins such as ORF2p and/or ORF1p.

Thereafter, the vector is transferred into cells cultured in vitro, tissues, organs, or administered to tissues, organs or organisms via pathways such as blood, lymph and cerebrospinal fluid, or local tissue administration through conventional means such as liposome transfection, so that the vector enters the cytoplasm of the target cell and binds to ORF1p and/or ORF2p before entering the nucleus, or ORF1p and/or ORF2p can mediate the vector to directly enter the nucleus. The vector RNA is connected to the short interspersed element RNA or its product transcribed in the cell and then binds to ORF2p expressed in the cell or binds to ORF2p and ORF1p at the same time; or directly binds to ORF2p expressed in the cell or binds to ORF2p and ORF1p at the same time (for example, when the Ω structure formed by the upstream sequence of the target site, the downstream sequence of the target site and the intermediate sequence to be inserted in the vector combines with the genome as the reverse transcription functional structure to replace the short interspersed element or its product to initiate reverse transcription), and the sequence to be inserted is inserted into the corresponding target site (site to be inserted) on the genome.

If the above method is continued according to the new site generated after insertion, the insertion can be continuous and the long fragment insertion without obvious length limit can be completed.

Since this method does not require mechanisms unique to eukaryotic systems, such as splicing mechanisms, it is suitable for systems that do not have eukaryotic pre-mRNA splicing mechanisms and cannot produce lariat structure, such as prokaryotes such as bacteria, and is also suitable for eukaryotic organisms that have pre-mRNA splicing mechanisms.

If directional transfer is required, the outer package of the vector can be modified. Pay attention to avoid RNA degradation during the whole process.

(3) RNA is used as a vector and the sequence is inserted into genome by a simple RNA framework containing a pan-ORF1p coding sequence and/or a pan-ORF2p coding sequence, or the RNA framework is connected downstream to one or more ORF2p functional initiation parts containing a pan-ORF1p coding sequence and/or a pan-ORF2p coding sequence (in order to minimize the impact on the receiving system, the types of short interspersed element RNA, short interspersed element derivative RNA, long interspersed element RNA, long interspersed element derivative RNA, ORF1p and/or ORF2p in the corresponding receiving system can be selected):

The above two methods add pan-ORF1p coding sequence and/or pan-ORF2p coding sequence upstream of the upstream sequence of the target site, downstream of the downstream sequence of the target site, upstream or downstream of the ORF2p functional initiation parts, and are located before, between, after or within each component (Do not affect the formation of “0” by the RNA framework at the target site) (upstream and downstream sequences of the site to be inserted (i.e., the target site) (within 20,000 bp respectively), add the sequence to be inserted (within 20,000 bp) at the insertion point between the upstream and downstream sequences), and produce the above-mentioned RNA as a vector.

The RNA can be placed in a solution containing an RNase inhibitor and/or an appropriate amount of Mg²⁺ (such as 6 mmol/L) or other metal ions or in a cell solution to promote the correct folding of the RNA and promote the subsequent binding with corresponding functional proteins such as ORF2p and/or ORF1p.

Thereafter, the vector is transferred into cells cultured in vitro, tissues, organs, or administered to tissues, organs or organisms via pathways such as blood, lymph and cerebrospinal fluid, or local tissue administration through conventional means such as liposome transfection, so that the vector is allowed to enter the target cell cytoplasm to express and produce ORF1p and/or ORF2p and bind to it, or it binds to ORF1p and/or ORF2p expressed in vivo and then enters the nucleus, or ORF1p and/or ORF2p can mediate the vector to directly enter the nucleus.

The vector RNA is connected to the short interspersed element RNA or its product transcribed in the cell and then binds to ORF2p expressed in the cell or encoded by vector or binds to ORF2p and ORF1p at the same time; or directly binds to ORF2p expressed in the cell or encoded by vector or binds to ORF2p and ORF1p at the same time (for example, when the Ω structure formed by the upstream sequence of the target site, the downstream sequence of the target site and the intermediate sequence to be inserted in the vector combines with the genome to replace the reverse transcription functional structure in the short interspersed element or its product to initiate reverse transcription), or with the help of one or more ORF2p functional initiation part(s) that have been connected to the downstream of the RNA framework combined with the ORF2p expressed by cell itself in the cell or encoded by the vector, the sequence to be inserted is inserted into the corresponding target site (site to be inserted) on the genome.

If the above method is continued according to the new site generated after insertion, the insertion can be continuous and the long fragment insertion without obvious length limit can be completed. If directional transfer is required, the outer package of the vector can be modified. Pay attention to avoid RNA degradation during the whole process.

(4) RNA and/or RNP comprising one or more ORF2p functional initiation part(s), one or more pan-ORF1p coding sequence(s), and/or one or more pan-ORF2p coding sequence(s), and/or DNA expressing one or more ORF2p functional initiation part(s), ORF1p, ORF2p, ORF1p-derivative proteins and/or ORF2p-derivative proteins, together with RNA vector of simple RNA framework, RNA vector with one or more ORF2p functional initiation part(s) connected downstream of RNA framework, and/or RNA vector with one or more pan-ORF1p coding sequence(s) and/or one or more pan-ORF2p coding sequence(s) connected downstream of RNA frame, or RNA vector with one or more ORF2p functional initiation part(s) connected downstream of RNA frame and one or more pan-ORF1p coding sequence(s) and/or one or more pan-ORF2p coding sequence(s), are administered to the target system in the same vector and/or different vectors (in order to minimize the impact on the receiving system, the types of short interspersed element RNA, short interspersed element derivative RNA, long interspersed element RNA, long interspersed element derivative RNA, ORF1p and/or ORF2p in the corresponding receiving system can be selected):

The RNA vector in “1-3” in the above “1. Genome sequence insertion technology using RNA as a vector” can be administered to the target system with RNA and/or RNP of one or more ORF2p functional initiation part(s), one or more pan-ORF1p coding sequence(s), and/or one or more pan-ORF2p coding sequence(s), and/or DNA expressing one or more ORF2p functional initiation part(s), ORF1p, ORF2p, ORF1p derived protein(s) and/or ORF2p derived protein(s) on the same vector or different vectors to exert their effects.

RNA containing one or more ORF2p functional initiation part(s) or the corresponding RNA expressed by DNA expressing one or more ORF2p functional initiation part(s) can be connected to various RNA vectors and/or RNA frameworks in vivo with or without cutting, and play the above-mentioned role to insert the sequence to be inserted into the target site.

The corresponding protein expressed by RNA containing one or more long interspersed element RNA(s), one or more pan-ORF1p coding sequence(s), and/or one or more pan-ORF2p coding sequence(s) or the corresponding protein expressed by DNA expressing long interspersed element RNAs, ORF2p, ORF1p, ORF2p-derived proteins and/or ORF1p-derived proteins can function in the above method to insert the sequence to be inserted into the target site.

If the above method is continued according to the new site generated after insertion, the insertion can be continuous and the long fragment insertion without obvious length limit can be completed.

If directional transfer is required, the outer package of the vector can be modified. Pay attention to avoid RNA degradation during the whole process.

The RNA can be placed in a solution containing an RNase inhibitor and/or an appropriate amount of Mg²⁺ (such as 6 mmol/L) or other metal ions or in a cell solution to promote the correct folding of the RNA and promote the subsequent binding with corresponding functional proteins such as ORF2p, ORF1p, ORF2p-derived proteins and/or ORF1p-derived proteins.

### II. Genome Sequence Insertion Technology Using RNP as a Vector

(in order to minimize the impact on the receiving system, the types of short interspersed element RNA, short interspersed element derivative RNA, long interspersed element RNA, long interspersed element derivative RNA, ORF1p and/or ORF2p in the corresponding receiving system can be selected):

First, various types of RNA vectors described in the above “Scheme 1” are prepared, and the proteins ORF2p, ORF1p, ORF2p-derivative proteins and/or ORF1p-derivative proteins are expressed and extracted and purified in vitro through a eukaryotic system or a prokaryotic system. The prepared RNA vector is mixed and incubated (Incubate at an appropriate temperature, either room temperature or 37° C., for less than 48 hours. The concentration of metal ions such as Mg2+ can be appropriately increased to promote the correct folding of the secondary and higher structures of the RNA vector) in vitro with cytoplasm containing ORF2p, ORF1p, ORF2p-derivative protein and/or ORF1p-derivative protein, or physiological fluid containing ORF2p, ORF1p, ORF2p-derivative protein and/or ORF1p-derivative protein to obtain an RNP vector.

Thereafter, the RNP vector is transferred into cells cultured in vitro, tissues, organs, or administered to tissues, organs or organisms via pathways such as blood, lymph and cerebrospinal fluid, or local tissue administration through conventional means such as liposome transfection,

The vector or expression sequence carrying ORF2p, ORF1p, ORF2p-derivative protein and/or ORF1p-derivative protein can still express ORF2p, ORF1p, ORF2p-derivative protein and/or ORF1p-derivative protein after entering the target cytoplasm, and continue to bind with them when the binding is not sufficient in vitro, or continue to bind to ORF2p, ORF1p, ORF2p-derivative protein and/or ORF1p-derivative protein (It can also bind to ORF2p, ORF1p, ORF2p-derivative protein and/or ORF1p-derivative protein simultaneously) expressed in vivo and then enter the nucleus, or ORF1p and/or ORF2p can mediate the direct nuclear entry of the vector. If the binding to ORF2p, ORF1p, ORF2p-derivative protein and/or ORF1p-derivative protein in vitro is insufficient, the various RNA vectors may continue to function in vivo.

The vector RNA is connected to the short interspersed element RNA or its product transcribed in the cell and then binds to ORF2p expressed in the cell or encoded by vector or binds to ORF2p and ORF1p at the same time; or directly binds to ORF2p expressed in the cell or encoded by vector or binds to ORF2p and ORF1p at the same time, or with the help of one or more ORF2p functional initiation part(s) that have been connected to the downstream of the RNA framework combined with the ORF2p expressed by cell itself in the cell or encoded by the vector, the sequence to be inserted is inserted into the corresponding target site (site to be inserted) on the genome. If the above method is continued according to the new site generated after insertion, the insertion can be continuous and the long fragment insertion without obvious length limit can be completed. If directional transfer is required, the outer package of the vector can be modified. Pay attention to avoid RNA degradation during the whole process.

III. RNA Vector and/or RNP Vector-Mediated Genome Sequence Deletion Technology

(1) Deletion of any region in the genome: The sequence to be inserted in the RNA and/or RNP vector designed in the above insertion technology is (“1. Genome sequence insertion technology using RNA as a vector” and “2. Genome sequence insertion technology using RNP as a vector”) changed to a certain sequence (within 20,000 bp) upstream or downstream (within 100,000 bp) of the insertion point (If the inserted sequence undergoes homologous recombination with its upstream sequence, any sequence can be inserted between the corresponding sequence of the sequence to be inserted and the sequence upstream of the target site on the genome, which will not affect the result or can promote subsequent homologous recombination and/or its effect; if the inserted sequence undergoes homologous recombination with its downstream sequence, any sequence can be inserted between the corresponding sequence of the sequence to be inserted and the sequence downstream of the target site on the genome, which will not affect the result or can promote subsequent homologous recombination and/or its effect). Through the RNP or RNA-mediated genome sequence insertion approach (“1. Genome sequence insertion technology using RNA as a vector” and “2. Genome sequence insertion technology using RNP as a vector”) described in the present invention, the sequence between two identical sequences can be removed with a certain efficiency through homologous recombination after the insertion of the sequence. Sequences containing recombination sites (GCAGA[A/T]C, CCCA[C/G]GAC and/or CCAGC) can be selected for insertion to increase the efficiency of subsequent homologous recombination. If directional transfer is required, the outer package of the vector can be modified. Pay attention to avoid RNA degradation during the whole process. In addition, if the sequence to be removed is 600 bp or less, it is possible to delete the corresponding fragment through homologous recombination and/or other genome repair mechanisms with high efficiency.

(2). Deletion from the CNV end: Under physiological conditions, copy number variation (CNV) is similar to a copy of the original complete gene. Through the above mechanism, the CNV as a copy can be continuously extended according to the original complete gene, causing the protein expression and various states of cells, tissues and organisms to change continuously.

The CNV end is composed of the upstream gene part (portion) and the downstream partial short interspersed element (ORF2p functional initiation part) part, and the short sequence fragment formed by the connection of the lariat structure and the partial short interspersed element (ORF2p functional initiation part) will be continuously inserted between these two parts to extend the CNV. In the early embryonic development, the transcription of the long interspersed elements increases significantly, while the short interspersed elements on the genome, such as Alu sequences, shows obvious demethylation. While the long interspersed element-mediated 3′ transduction (based on the right monomer missing of the short interspersed elements upstream of the promoter and the complete short interspersed element structure downstream) initiates the extension of the related gene copy number variation (CNVs), the demethylated short interspersed element sequences undergo homologous recombination with each other, deleting most of the previously extended CNVs (initialization).

After that, the fully initialized embryonic cells resume the high methylation state, and the ends of the CNVs are gradually extended mediated by partial short interspersed element at the ends of the CNVs, thereby changing the expression and state of each cell. The gene expression of each cell in turn affects the CNVs changes through the lariat structure, thus changing the genome and gradually inducing differentiation. This is consistent with the common CNVs changes in embryos and the CNVs differences in various tissues.

The extension of CNVs of different genes is common in various tumor cells and is positively correlated with clinical grading. At the same time, the expression levels of proto-oncogenes and tumor suppressor genes are also proportional to the length of CNVs, so the formation and progression of tumors should be related to the disorder of CNVs of proto-oncogenes or tumor suppressors. In addition, some irreversible diseases related to external stimuli, such as diabetes, may also be related to the disorder of CNVs. Since most drug resistance is related to the change in expression of the corresponding protein caused by long-term external stimulation, it may involve the CNV change of the corresponding gene, which can also be improved or hindered by the present invention.

The CNV ends in cells or tissues are detected by sequencing and alignment (alignment to the junction of the gene sequence and the partial short interspersed element), and the corresponding RNA sequence of the gene portion (within 2000 bp) in the CNV end to be processed and/or the 3′ part sequence of the lariat that can be formed by the downstream sequence of the CNV end within a range (within 200000 bp) in the complete gene (The lariat that can be formed downstream can be predicted or detected by the following method) (Alternatively, the sequence within the downstream range (within 200,000 bp) of the complete gene can be directly selected and truncated to replace the above 3′ partial sequence) are selected as the upstream sequence of the target site, and the corresponding RNA sequences of the sequence immediately upstream (within 100000 bp) of the sequence to be deleted of the end (Any sequence can be inserted between the sequence within 100,000 bp upstream of the end sequence to be deleted and the portion of the sequence upstream of the target site, which will not affect the result or may promote subsequent homologous recombination and/or its effect) are connected respectively as the sequence to be inserted, and then the complete short interspersed element RNA, partial short interspersed element RNA or short interspersed element-like RNA (i.e. the ORF2p functional initiation part) (according to the different insertion methods mentioned above) (according to the above, it can be followed by ORF1p and ORF2p coding sequences) is connected as the downstream sequence of the target site, for synthesis, and through one of the above-mentioned gene insertion methods (through RNA vector or RNP vector), the sequence immediately upstream of the sequence to be deleted of the end is inserted between the gene portion and the partial short interspersed element sequence (The efficiency can be improved if the short interspersed element sequence used on the vector is the same as or close to the short interspersed element sequence around the insertion point) of the actual CNV end, and then the sequence to be deleted is deleted by homologous recombination between the same sequences.

Sequences containing recombination sites (GCAGA[A/T]C, CCCA[C/G]GAC and/or CCAGC) can be selected for insertion to increase the efficiency of subsequent homologous recombination. If directional transfer is required, the outer package of the vector can be modified. Pay attention to avoid RNA degradation during the whole process.

Multiple corresponding RNA, RNP and/or DNA with gene editing function can be administered at the same time to delete the CNV ends of multiple genes or multiple different CNV ends of a gene at the same time. This method can modify the state of cells, such as the state of the tumor, such as the tumor grade, or the differentiation of the cells, etc., because it can change the expression of genes and cell epigenetics.

(3). Continuous deletion of CNV ends: If the corresponding RNA, RNP and/or DNA with gene editing effect in the present invention is administered according to the above-mentioned CNV end deletion method to delete the secondarily generated CNV ends at the same time as the above-mentioned CNV end deletion is performed or thereafter, the continuous action of this process can cause the CNV of the corresponding gene to be continuously deleted.

A variety of corresponding RNAs, RNPs and/or DNAs with gene editing effects can be administered simultaneously to delete the CNV ends of multiple genes or multiple different CNV ends of a gene simultaneously or sequentially.

This method can modify the state of cells, such as the state of the tumor, such as the tumor grade, or the differentiation of the cells, etc., because it can change the expression of genes and cell epigenetics.

IV. RNA Vector and/or RNP Vector-Mediated Genome CNV End Extension or CNV Addition Technology

(1). Addition of new CNVs to the genome: Using any position on the genome as the target site, one or more copies of a gene can be added according to the gene editing method of the present invention. The method can search for or (and) select sequences containing recombination sites (GCAGA[A/T]C, CCCA[C/G]GAC and/or CCAGC), short interspersed element sequences, partial short interspersed elements and/or short interspersed element derivatives (ORF2p functional initiation part) on the genome to be edited as the corresponding upstream and/or downstream sequences of the target site on the genome for sequence insertion of gene copies, thereby improving the corresponding gene editing effect by increasing the efficiency of homologous recombination.

Adding recombination sites (GCAGA[A/T]C, CCCA[C/G]GAC and/or CCAGC), short interspersed element RNA, partial short interspersed element RNA and/or short interspersed element derivative RNA in the upstream and/or downstream sequences of the target site in the RNA framework can increase the corresponding gene editing effect. The ends of the newly added gene copies (i.e., the newly generated CNV ends) can be extended or shortened for further gene editing.

The sequence of recombination sites (GCAGA[A/T]C, CCCA[C/G]GAC and/or CCAGC), short interspersed element sequences, partial short interspersed elements and/or short interspersed element derivatives (The corresponding sequence of the ORF2p functional initiation part) can be searched on the genome as the corresponding sequence of the downstream sequence of the target site on the genome and a new gene copy can be inserted through it, thereby improving efficiency, especially using partial short interspersed element RNA as the downstream sequence of the target site, which is more consistent with the downstream sequence of the CNV end in its natural state.

A variety of corresponding RNAs, RNPs and/or DNAs with gene editing effects can be administered simultaneously to add CNVs of multiple genes or multiple CNVs of different lengths and states of a gene simultaneously or sequentially.

This method can modify the state of cells, such as the state of the tumor, such as the tumor grade, or the differentiation of the cells, etc., because it can change the expression of genes and cell epigenetics.

(2). Addition and extension of CNV ends on the genome: Addition and extension of existing CNV ends on the genome. According to the aforementioned sequence insertion method of the present invention, the upstream part (gene part) of the CNV end is used as the corresponding sequence of the upstream sequence of the target site on the genome, the downstream part (partial short interspersed elements (ORF2p functional initiation part)) of the CNV end is used as the corresponding sequence of the downstream sequence of the target site on the genome, and the downstream sequence of the upstream part (gene part) of the CNV end in the complete gene sequence is used as the corresponding sequence of the sequence to be inserted on the genome, and the CNV end of the corresponding gene is extended.

A variety of corresponding RNA, RNP and/or DNA with gene editing effects can be administered simultaneously to simultaneously add and/or extend the CNV ends of multiple genes or multiple different CNV ends of a gene.

This method can modify the state of cells, such as the state of the tumor, such as the tumor grade, or the differentiation of the cells, etc., because it can change the expression of genes and cell epigenetics.

### V. Genome Sequence Replacement Technology (Including Sequence Replacement, Site Deletion, Site Addition, Sequence Addition, Sequence Deletion and Site Replacement)

The sequence to be inserted in the vector designed in the above insertion technology is changed to the sequence to be replaced (replacement sequence) and the surrounding sequence of the sequence to be replaced on the genome (that is, the DNA sequence of the replacement sequence to be inserted and the sequence on the genome that will be deleted after homologous recombination occurs, and whether it is located at 3′ or 5′ of the replacement sequence when constructing the vector depends on whether the insertion site is upstream or downstream of the sequence to be replaced on the genome) (the DNA sequence of the replacement sequence should be homologous to the sequence to be replaced on the genome) (if the inserted sequence undergoes homologous recombination with its upstream sequence, any sequence can be inserted between the corresponding sequence of the sequence to be inserted on the genome and the corresponding sequence of the upstream sequence of the target site, which does not affect the result or can promote subsequent homologous recombination and/or its effect; if the inserted sequence undergoes homologous recombination with its downstream sequence, any sequence can be inserted between the sequence to be inserted on the genome and the downstream sequence of the target site, which does not affect the result or can promote subsequent homologous recombination and/or its effect), and the replacement sequence and the surrounding sequence of the sequence to be replaced on the genome are inserted upstream or downstream of the sequence to be replaced on the genome through the above gene editing insertion method, when the inserted replacement sequence undergoes homologous recombination with the sequence to be replaced on the genome, the sequence to be replaced on the genome is replaced by the inserted replacement sequence that is homologous to it. At the same time, the surrounding sequence of the sequence to be replaced deleted due to homologous recombination is reinserted together with the replacement sequence when the sequence is inserted.

Replacements on the genome include sequence replacement and site replacement. Sequence replacement means that the replacement sequence to be inserted has some sequences, such as one or several inconsistencies, between the corresponding sequence on the genome. Site replacement means that the replacement sequence to be inserted has some sites, such as one or several inconsistencies, between the corresponding sequence on the genome. Site deletion means that the replacement sequence to be inserted has some sites, such as one or several deletions, compared with the corresponding sequence on the genome. Site addition means that the replacement sequence to be inserted has some sites, such as one or several additions, compared with the corresponding sequence on the genome. Sequence addition means that the replacement sequence to be inserted has some sequences, such as one or several additions, compared with the corresponding sequence on the genome. Sequence deletion means that the replacement sequence to be inserted has some sequences, such as one or several deletions, compared with the corresponding sequence on the genome.

The smaller the difference between the replacement sequence to be inserted and the corresponding homologous sequence on the genome, the higher the efficiency; the inconsistency between the replacement sequence to be inserted and the corresponding homologous sequence on the genome should be avoided as much as possible at or near the two ends or both sides of the replacement sequence to be inserted to improve efficiency.

If directional transfer is required, the outer package of the vector can be modified. Pay attention to avoid RNA degradation during the whole process.

VI. RNA Vector and/or RNP Vector-Mediated Genome Sequence Deletion with Simultaneous Sequence Insertion, Sequence Replacement or Site Replacement

By adding the sequence desired to be inserted into the genome after a certain sequence (within 20,000 bp) upstream or downstream (within 100,000 bp) of the insertion site in the above-mentioned “RNA vector and/or RNP vector-mediated genome sequence deletion technology” operation, the sequence desired to be inserted into the genome can be inserted upstream or downstream of the deleted sequence while deleting the target sequence.

By replacing a certain sequence within 20,000 bp upstream or downstream (within 100,000 bp) of the insertion site (target site) in the above-mentioned “RNA vector and/or RNP vector-mediated genome sequence deletion technology” operation with a sequence that is different from a (this) certain sequence within 20,000 bp upstream or downstream (within 100,000 bp) of the insertion site (target site) in terms of site or partial sequence (the different site and/or partial sequence is the site and/or sequence to be replaced), the site or partial sequence in the upstream or downstream sequence of the deleted sequence can be replaced with the site and/or partial sequence to be replaced while deleting the target sequence.

VII. Technologies that Block Genome Changes Caused by Transposons and Stabilize the Genomes and CNVs Thereof

(That is, through this gene editing technology, a sequence that is inconsistent or non-homologous to the genome or the gene part in the CNV end and its upstream, downstream or upstream and downstream sequences in the complete gene is inserted between the gene part and the partial short interspersed element sequence or other regions at the CNV end, thereby hindering the further extension of the CNV; the CNV end is defined as the place where the gene sequence is directly connected to the partial short interspersed element sequence or other related sequences (the corresponding DNA sequence of the ORF2p functional initiation part), where the gene can be extended, and the specific sequence of the gene sequence and the partial short interspersed element sequence or other related sequences (the corresponding DNA sequence of the ORF2p functional initiation part) at each specific CNV end can be obtained through molecular biological methods such as gene sequencing or gene chips.)(Through conventional transfection methods such as the use of fat-soluble substances or substances with cell transfection ability such as liposomes, the corresponding carriers are encapsulated and transferred into cells, tissues, organs cultured in vitro, or into tissues, organs or organisms through pathways such as blood, lymph and cerebrospinal fluid, or local tissue administration.)(The RNA used below can be replaced by a DNA vector that can express the corresponding RNA, so that it is expressed in the corresponding target system to produce RNA and/or RNP produced by the RNA combining with ORF1p, ORF2p, ORF1p-derivative protein and/or ORF2p-derivative protein produced by the DNA vector; the RNA used below can also be replaced by RNP formed by the corresponding RNA combining with ORF1p, ORF2p, ORF1p-derivative protein and/or ORF2p-derivative protein when possible or necessary.)

(1). Intervention of a specific CNV (where the upstream sequence used for insertion is the gene part at the CNV end of a specific gene): Select the CNV to be operated, and set the junction between the 3′ end of its gene part and the partial short interspersed element sequence or other related sequence (the corresponding DNA sequence of the ORF2p functional initiation part) as the insertion site (target site). Set the upstream sequence of the insertion site (target site) in the above insertion method to the corresponding RNA sequence at the 3′ end (within 20,000 bp) of the gene part at the CNV end, and the downstream sequence is the partial short interspersed element RNA or other related sequence (the ORF2p functional initiation part)(Therefore, the short interspersed element RNA, partial short interspersed element RNA or short interspersed element-like sequence (ORF2p functional start part) connected to the downstream sequence of the target site in the above method can be omitted). The sequence to be inserted is any sequence that is not homologous to the genome or the gene part at the CNV end and its upstream and downstream sequences in the complete gene (within 20,000 bp). After the vector is constructed, it is transferred into the corresponding cells, living tissues or organisms through the above-mentioned RNA vector and/or RNP vector pathway, so that the non-homologous sequence is inserted into the corresponding CNV end. Since the non-homologous sequence does not exist downstream of the gene sequence of the corresponding CNV end in the complete gene, it is impossible to further extend the CNV end based on the complete gene sequence, thereby hindering further changes in the CNV end.

(2). Intervention of CNVs across the genome (the upstream sequence of the target site used for insertion needs to include all possible gene parts at the CNV end):

1. Genome fragmented sequence method: cells from the organism, tissue or cell line to be operated are cultured in vitro, or the genome is directly extracted, and enriched using random primers and PCR after ultrasonic fragmentation; short random sequences (within 20 bp) are designed and synthesized, and partial short interspersed element sequences are connected downstream. The amplified genomic fragments were connected to the synthesized short random sequence connected to the partial short interspersed element sequence fragment by PCR and amplified to obtain different genomic fragment sequences connected to the random sequence and then connected to the partial short interspersed element sequence or other related sequences (the corresponding DNA sequence of the ORF2p functional initiation part). The obtained fragments are constructed to generate corresponding RNA, which is transferred into corresponding cells, living tissues or organisms through the above-mentioned RNA vectors and/or RNP vectors, and all CNV ends on the genome are targeted via the genome fragmented sequence, so that the CNV ends are inserted into non-homologous sequences between their gene parts and the partial short interspersed elements (i.e., a short random sequence or a portion of a short random sequence, the portion of which that is not homologous to the gene fragment is non-homologous to the local gene sequence of the corresponding gene fragment), since the non-homologous sequence does not exist downstream of the corresponding CNV end gene partial sequence in the complete gene, further changes in the CNV end are hindered.

2. Random sequence method: Generate a random sequence of appropriate length (within 100 bp) (including all possible permutations, excluding combinations similar to short interspersed element sequences) and connect it to any non-homologous sequence (within 20,000 bp) in the genome and then connect it to partial short interspersed element RNA or other related sequences (the ORF2p functional initiation part); Or it contains random sequences (within 100 bp) connected to some short interspersed element RNA or other related sequences (the ORF2p functional initiation part), in which some short interspersed element RNA or other related sequences (the ORF2p functional initiation part) are added with any sequence (within 2000 bp) that is not homologous to the genome after the middle natural splicing site (For example, for the transcription product of Alu, it is the splicing site in the middle that can be spliced to produce scAlu and partial Alu); Or synthesize and produce RNA that a random sequence is followed by any non-homologous sequence to the genome (thereafter expressed as a lariat), and at the same time construct a vector that transcribes short interspersed element RNA and/or partial short interspersed element RNA, and the short interspersed element RNA can be connected downstream or additionally express a DNA sequence or RNA sequence of a long interspersed element sequence or its protein coding sequence corresponding to the function of the short interspersed element (or directly introduce the RNA of the short interspersed element and/or partial short interspersed element into the target system). The above-mentioned vector is transferred into corresponding cells, living tissues or organisms, and all CNV ends on the genome are targeted by random sequences according to the above-mentioned mechanism and method, so that non-homologous sequences are inserted into the corresponding CNV ends. Since the non-homologous sequence does not exist downstream of the gene part of the corresponding CNV end in the complete gene, further changes in the CNV end are hindered.

3. Lariat end sequence method: detect all lariat types (A short random sequence (within 100 bp) that is non-homologous to the genome is inserted into the short interspersed element sequence, and the short interspersed element RNA expressed by it can still be normally cleaved into a partial short interspersed element (that is, the insertion position of the non-homologous sequence is downstream of the natural cleavage site of the short interspersed element and is not located at the cleavage site), and a plasmid that can express the modified short interspersed element RNA is constructed and transferred into cells amplified from the corresponding organism to be operated or cell lines of the corresponding species (Alternatively, the genome of the corresponding species to be tested can be taken and the whole genome cut into fragments with a longer length (more than 200 bp) and a certain degree of overlap (overlapping more than 10 bp), and then constructed into a vector and overexpressed in the in vitro cells of the corresponding species through RNA polymerase II). After a period of time, the corresponding nucleic acid is extracted according to the sequence specificity of the non-homologous sequence inserted into the short interspersed element RNA and sequenced, and the sequence information of various generated lariats connected to the partial short interspersed element integrated with the non-homologous sequence is obtained) and/or predict the lariat sequence based on the sequence rules of pre-mRNA forming lariat (for example, most of them end with AG), and obtain all lariat sequence information of the species or individual. Synthesize the the RNAs containing the 3′ sequence (within 20,000 bp) of each lariat connected of any sequence (within 20,000 bp) that is not homologous to the genome, and generate RNA of short interspersed elements at the same time (which can be followed by the long interspersed element sequence or its protein coding sequence corresponding to the function of the short interspersed element to increase efficiency as described above), and introduce them into the target system together; Or generate RNA in which each obtained lariat 3′ sequence is connected to any non-homologous sequence (within 2000 bp) in the genome and then connected to partial short interspersed elements (According to the above, it can be followed by a long interspersed element sequence or its protein coding sequence corresponding to the function of the short interspersed element to increase efficiency) (The SINE sequence is preferably the same or similar to the SINE sequence in the gene where the lariat 3′ sequence connected to it is located to increase efficiency). The above-mentioned RNP vector or RNA vector is introduced into the corresponding cells, tissues or organisms, and the CNV ends in the whole genome are edited.

4. Short interspersed element sequence modification method: by additionally administering modified short interspersed element RNA, a sequence that is non-homologous to the genome or to the gene part at the CNV end and its upstream and downstream sequences in the complete gene is inserted into each CNV end, thereby hindering the extension of the CNV end.

Generate RNA containing a complete short interspersed element sequence (A long interspersed element sequence or its protein coding sequence corresponding to the function of the corresponding type of short interspersed element can be added behind it to increase efficiency) with an additional short sequence (Inconsistent with the 3′ sequence of the conventionally generated lasso, a short sequence spanning the natural splicing site of the short element is sufficient (within 100 bp)) added before the natural splicing site of the short interspersed element RNA so that the transcription product (RNA) of the short interspersed element can also be naturally spliced in the additional region. Alternatively, a complete short interspersed element RNA (A long interspersed element sequence or its protein coding sequence corresponding to the function of the corresponding type of short interspersed element can be added behind it to increase efficiency) with any sequence non-homologous to the genome (within 200 bp) added after the natural splicing site of the short interspersed element transcription product or RNA containing this RNA sequence is produced, and then administered to the corresponding cells, living tissues or organisms. The short interspersed element sequences used should cover as much as possible all the short interspersed element sequences of the species or individual (which can be obtained through sequencing or array chips, etc.) to accurately modify all CNV ends in the entire genome.

The whole genome can also be cut into long fragments that overlap with each other to a certain extent (the overlapping length is longer than the length of a lariat structure), and the RNA of the long fragment is produced and transferred into the in vitro cell line of the corresponding species to produce a lariat structure. Thereafter, the corresponding RNA of the modified short interspersed element sequence (Long interspersed element sequences or their protein coding sequences corresponding to the functions of short interspersed elements of the corresponding types are added downstream, and can be mediated by RNA pathway) produced above is transferred, and then the biologically active single-stranded RNA ribonucleoprotein complex (RNP) or RNA with the partial short interspersed elements (produced by the modified short interspersed elements) connected to the produced lariat is separated and purified through sequence specificity and other properties and conventional means, and then exerts its effect through the corresponding RNA or RNP pathway.

(3). Modification of the ORF2p functional initiation part on the genome: inserting any sequence (within 500 bp) into the transcription-related non-coding regions of short interspersed elements, short interspersed element derivatives, or initiating ORF2p splicing and reverse transcription functional structure on the genome, such as cis-acting elements such as promoters, enhancers, regulatory sequences, or inducible elements, the transcription regions therein, the natural splicing sites of transcription products, or other sequences of short interspersed elements, short interspersed element derivatives, or initiating ORF2p splicing and reverse transcription functional structure, and/or the transcription-related non-coding regions of long interspersed elements, long interspersed element derivatives, such as cis-acting elements such as promoters, enhancers, regulatory sequences, or inducible elements, the transcription regions therein, protein coding sequences, or other sequences, through the present invention, this makes it impossible for short interspersed element sequences, short interspersed element derivative sequences, and initiating ORF2p splicing and reverse transcription functional structure in the genome to be transcribed or spliced after transcription, and/or makes it impossible for long interspersed element sequences or long interspersed element derivatives to be transcribed or produce proteins with normal functions.

First, the short interspersed elements, long interspersed elements, short interspersed element derivatives, long interspersed element derivatives, initiating ORF2p splicing and reverse transcription functional structure, and related regions such as non-coding regions related to the transcription of the corresponding sequences, such as promoters, enhancers, regulatory sequences or inducible elements, and other cis-acting element sequences of the whole genome of the individual to be operated are sequenced to obtain the corresponding sequences. Select its transcription-related non-coding regions such as cis-acting elements such as promoters, enhancers, regulatory sequences or inducible elements, transcription regions therein, natural splicing sites of transcription products, protein coding sequences or other sequences as target sites. The upstream and downstream sequences of the target site in the invention are short interspersed elements, long interspersed elements, short interspersed element derivatives, long interspersed element derivatives, initiating ORF2p splicing and reverse transcription functional structure relative to the upstream and downstream sequences of the target site, and the sequence to be inserted is an arbitrary sequence.

The above insertion method is used to insert any sequence into the corresponding site of short interspersed elements, long interspersed elements, short interspersed element derivatives, long interspersed element derivatives, or initiating ORF2p splicing and reverse transcription functional structure on the genome. In addition, the above gene editing method can be used to replace (sequence replacement, site deletion, site addition, sequence addition, sequence deletion and/or site replacement) or delete short interspersed elements, long interspersed elements, short interspersed element derivatives, long interspersed element derivatives, or initiating ORF2p splicing and reverse transcription functional structure on the genome to make them inactive or reduce their function.

(4). Delete and fix the CNV end: Select the CNV end to be operated, set the junction of the 3′ end of its gene part and partial short interspersed elements or other related sequences (the corresponding DNA sequence of the ORF2p functional initiation part) as the target site, and set the 3′ end of the gene part of the CNV end (within 2000 bp) as the upstream sequence of the target site in the above insertion method, and the downstream sequence of the target site is the partial short interspersed element RNA or other related sequences (the ORF2p functional initiation part)(Therefore, the partial short interspersed element connected to the downstream sequence of the target site in the above method can be omitted).

The sequence to be inserted is the corresponding RNA sequence of the sequence (within 20,000 bp) immediately upstream of the sequence to be deleted (within 100,000 bp) on the genome, followed by an arbitrary sequence (within 20,000 bp) that is not homologous to the genomic sequence (any sequence can be inserted between the corresponding sequence of the sequence to be inserted on the genome and the corresponding sequence of the upstream sequence of the target site, which does not affect the result or can promote subsequent homologous recombination and/or its effect).

After the above-mentioned RNA or RNP vector is produced, it is transferred into the corresponding cells, living tissues or organisms through the above-mentioned RNA or RNP pathway, so that the sequence immediately upstream of the sequence to be deleted on the genome followed by the non-homologous sequence is inserted into the corresponding CNV end. When homologous recombination occurs between the two identical sequences, the intermediate sequence is deleted, and the non-homologous sequence inserted will simultaneously hinder the further extension of the CNV.

(5). Inhibition of inherent mechanism: It is also possible to directly inhibit the CNV extension mechanism inherent to cells or organisms, for example, by inhibiting the transcription of short interspersed elements, long interspersed elements, short interspersed element derivatives, long interspersed element derivatives or other related sequences (the corresponding DNA sequence of the ORF2p functional initiation part) or the production of their RNA and the proteins encoded therein, such as ORF1p, ORF2p, ORF1p-derivative proteins or ORF2p-derivative proteins, by binding specific proteins to proteins related to the CNV extension mechanism, such as ORF1p, ORF2p, ORF1p-derivative proteins or ORF2p-derivative proteins or spliceosomes, or the functional structure of the complex, the function of the CNV extension mechanism is hindered, through the above-mentioned gene editing technology, short interspersed elements such as Alu and various MIRs, short interspersed element derivatives, various long interspersed elements and long interspersed element derivatives, and the corresponding protein coding sequences therein are modified to inactivate or reduce their activity, inhibit the function of related proteins in homologous recombination or mismatch repair mechanism, or give modified nucleoside substances to hinder reverse transcription, thus nhibit the intrinsic CNV extension mechanism, thereby hindering genomic changes and stabilizing CNVs.

### VIII. Editing the Genome by Administering Long RNA or RNP:

Since RNA can produce overlapping lariats in sequences through the intracellular splicing mechanism in eukaryotic cells, in theory, these overlapping lariats are multiple RNA frameworks containing corresponding upstream sequence of target site, downstream sequence of target site, and sequence to be inserted. The downstream sequence of target site of an RNA framework contained in a lariat is the upstream sequence of target site of another RNA framework contained in a lariat that overlaps with it in sequence. Therefore, in the case where there is already a partial Alu sequence upstream connected to the upstream sequence of the sequence to be inserted in the RNA or RNP to be given in the genome. After giving a longer sequence of RNA or RNP, the sequence information on it can be gradually inserted into the genome, and finally complete the above-mentioned various gene editing tasks such as Genome insertion, genome deletion, genome sequence replacement, genome site replacement, genome sequence deletion and sequence insertion, sequence replacement or site replacement, hindering the genome changes caused by transposons and stabilizing the genome and CNVs on it, genome sequence insertion, genome deletion, genome sequence replacement, genome site replacement, genome sequence deletion and sequence insertion, sequence replacement or site replacement at the CNV end on the genome, etc.

### IX. Assisting Other Gene Editing Technologies:

Since the present invention can produce RNA, single-stranded and double-stranded DNA in the target system, these RNA, single-stranded and double-stranded DNA can provide templates for gene editing for other gene editing technologies, and can provide DNA templates for other gene editing technologies such as TALEN, ZFN, Targetron, CRISPR or CRISPR/Cas9 to cut the genome and perform gene editing (such as homologous recombination or other effects) such as inserting exogenous sequences (sequences to be inserted), thereby assisting and promoting the corresponding gene editing technologies.

The upstream and downstream sequences of the target site on the single-stranded or double-stranded DNA generated by the RNA, RNP and/or DNA vector with gene editing function in the present invention are complementary to the corresponding sequences on the genome, and the sequence to be inserted is inserted into the single-stranded or double-stranded DNA incision generated by cutting the target site on the genome by technologies such as TALEN, ZFN, Targetron, CRISPR or CRISPR/Cas9, thereby inserting the sequence to be inserted into the target site on the genome, assisting gene editing by technologies such as TALEN, ZFN, Targetron, CRISPR or CRISPR/Cas9 or improving its efficiency.

The present invention contains or can produce RNA containing a upstream sequence of target site, a sequence to be inserted, a downstream sequence of target site and/or other sequences such as ORF2p functional initiation part such as short interspersed element RNA, partial short interspersed element RNA, etc., and ssDNA and/or dsDNA reversely transcribed therefrom, these components can assist TALEN, ZFN, Targetron, CRISPR and CRISPR/Cas9 technologies in homologous recombination or inserting corresponding sequences into target sites, promote the application of RNA and non-viral transfection of corresponding technologies to a greater extent (RNA transduction into cells does not require nuclear entry, and can enter the nucleus during the non-division period under the binding and action of corresponding proteins such as ORF1p and/or ORF2p) and improve the efficiency of genomic sequence insertion of corresponding technologies.

In addition, a DNA vector expressing an RNA framework (containing a sequence upstream of the target site, a sequence to be inserted, a sequence downstream of the target site, and other sequences such as ORF2p functional initiation part such as short interspersed element RNA, partial short interspersed element RNA, etc.,) can continuously produce RNA containing the RNA framework, and with the help of the reverse transcription functional structure on the RNA, convert the RNA into single-stranded DNA or double-stranded DNA, so as to continuously provide DNA templates for other gene editing technologies such as TALEN, ZFN, Targetron, CRISPR or CRISPR/Cas9 to cut the genome and perform gene editing (such as homologous recombination or other effects), such as inserting exogenous sequences (sequences to be inserted), thereby assisting and promoting the corresponding gene editing technology.

At the same time, RNA containing an RNA framework (containing a sequence upstream of the target site, a sequence to be inserted, a sequence downstream of the target site, and other sequences such as ORF2p functional initiation part such as short interspersed element RNA, partial short interspersed element RNA, etc.,) can be administered to a target after binding to ORF2p and/or ORF1p in vitro, and ORF2p and/or ORF1p in the target body convert the RNA into single-stranded DNA and/or double-stranded DNA, thereby continuously providing DNA templates for gene editing (such as homologous recombination or other effects) performed after genome cutting by other gene editing technologies such as TALEN, ZFN, Targetron, CRISPR or CRISPR/Cas9, such as inserting exogenous sequences (sequences to be inserted), thereby assisting and promoting the effects of the corresponding gene editing technology.

At the same time, after expressing ORF2p and/or ORF1p in the target body, RNA containing the RNA framework (containing a sequence upstream of the target site, a sequence to be inserted, a sequence downstream of the target site, and other sequences such as ORF2p functional initiation part such as short interspersed element RNA, partial short interspersed element RNA, etc.,) can be administered to the target, and after ORF2p and/or ORF1p binds to the RNA in the target body, the RNA is converted into single-stranded DNA and/or double-stranded DNA, thereby continuously providing DNA templates for gene editing (such as homologous recombination or other effects) performed after genome cutting by other gene editing technologies such as TALEN, ZFN, Targetron, CRISPR or CRISPR/Cas9, such as inserting exogenous sequences (sequences to be inserted), thereby assisting and promoting the effects of the corresponding gene editing technology.

The RNA used in the present invention may be linear or circular. Circular RNA can be obtained by adding complementary sequences greater than 5 bp in length, such as Alu element sequences or intron sequences, on both sides of the RNA framework to produce circular RNA in vitro or in vivo and play the corresponding role in the present invention. The flanking sequences of the RNA framework are designed to allow intron self-splicing, and circular RNA containing the RNA framework can also be generated in vivo or in vitro. The flanking sequences of the RNA framework contain RNA binding protein (RBP) binding sites, and circular RNA containing the RNA framework can also be generated in vivo or in vitro.

The application of the related technology of the present invention can edit the copy number variation and the end (terminal) part of each gene, change the end position or stabilize the end, which determines the gene expression, thereby achieving the purpose of stabilizing or changing the various states of cells and organisms, and therefore can be used in transformation of the genes and states of cells, tissues and organisms, modification of organisms such as human genome to improve function, modification of organisms such as human genome to treat various gene-related genetic diseases such as Huntington's disease and fragile X syndrome, delay or stop the change of genes and states of cells and organisms, change the genes and states of cells or organisms, tissue and organ regeneration and biological regeneration, assisted reproduction by converting somatic cells into germ cells by introducing transcription factors, prevention or delay of neurodegenerative diseases such as Parkinson's disease, Alzheimer's disease, Huntington's disease, amyotrophic lateral sclerosis, multiple system atrophy, primary lateral sclerosis, spinocerebellar ataxia, Pick's disease, frontotemporal dementia, Lewy body dementia and progressive supranuclear palsy, inhibition of tumor cell metabolic activity, proliferation rate and production while delaying its deterioration and improving its malignancy, as well as research and treatment of all other diseases related to gene and CNVs changes such as diabetes and other physiological, pathological and pathophysiological research fields.

The present invention can prevent the occurrence of glioma, breast cancer, cervical cancer, lung cancer, gastric cancer, colorectal cancer, duodenal cancer, leukemia, prostate cancer, endometrial cancer, thyroid cancer, lymphoma, pancreatic cancer, liver cancer, melanoma, skin cancer, pituitary tumor, germ cell tumor, meningioma, meningioma, glioblastoma, various astrocytomas, various oligodendrogliomas, astrocytic oligodendrocytomas, various ependymomas, choroid plexus papilloma, choroid plexus carcinoma, chordoma, various ganglioneuromas, olfactory neuroblastoma, sympathetic nervous system neuroblastoma, pineal cell tumor, pinealoblastoma, medulloblastoma, trigeminal nerve sheath tumor, facial acoustic neuroma, Glomus jugular tumor, angioreticuloma, craniopharyngioma or granular cell tumor and its metastatic cancer, inhibit their proliferation and prevent their grade increase and progression, or reverse their characteristics; prevent, delay or improve drug resistance to insulin, levodopa, various tumor chemotherapy drugs and targeted drugs, delay or stop genetic and state changes of cells and organisms, tissue and organ regeneration and biological regeneration.

In the present invention, a deterministic sequence or site (such as the sequence to be inserted and the upstream sequence of target site, the downstream sequence of target site or the sequences on both sides of the target site on the genome) (the sequence is DNA or RNA) is defined along the 5′→3′ direction; upstream is before the 5′ end of the deterministic sequence or site, and downstream is after the 3′ end of the deterministic sequence or site; the upstream sequence is the sequence located before the 5′end of the deterministic sequence or site, and the downstream sequence is the sequence after the 3′ end of the deterministic sequence or site.

When designing RNA vector or RNP vector sequences, software (such as PCFOLD or RNAFOLD) can be used to simulate the secondary structure of the RNA vector or RNP vector, so that the upstream sequence of the target site, especially the free end or free part of the upstream sequence of the target site, is more in a single-stranded free state, fewer complementary sequences inside the RNA vector or RNP vector sequence, especially inside the free end, free part or part close to the free end of the upstream sequence of the target site, may improve the gene editing efficiency. In addition, designing the sequence so that the secondary structure of the designed sequence to be inserted RNA is closer to the secondary structure of some short interspersed element RNAs (such as some Alu)(For example, forming complementary double-stranded structures on both sides of the bottom gap of the Ω structure formed by the designed sequence, and/or mimicking other stem-loop structures or protruding structures in the secondary structure of some short interspersed element RNAs (such as some Alu), etc.) can improve the efficiency of gene editing (such as improving the efficiency of ORF2p).

The circular secondary structure and the higher structure in the “Ω” structure required for the function of ORF2p in the RNA framework and its improved form should be close to (mimic) the circular secondary and higher structure in the “Ω” structure in the SINE and LINE transcription products corresponding to the ORF2p used in nature to increase efficiency, including the stem-loop, bulge, and A-A binding close to the root of the two legs of the “Ω” structure and the double-stranded complementary structure formed, including the shape, length, and relative position of the corresponding secondary and higher structures with the root of the two legs of the “Ω” structure and the sequence similarity of the corresponding positions. The sequence, secondary and higher structures of the right leg of the “Ω” structure can also imitate the sequence, secondary and higher structures of the right leg of the SINE and LINE transcription products corresponding to the ORF2p used in nature to increase efficiency, including the stem loop, protrusion and double-stranded complementary structure, including the shape, length, relative position (calculated from the start of the right leg to the 3′ direction) and the sequence similarity of the corresponding positions of the corresponding secondary and higher structures. At the same time, designing the two bases closest to the two legs in the open ring structure between the two legs of the “Ω” structure as adenine (A) or other pairs of bases with mismatched weak binding can stabilize the “Ω” structure to a certain extent, thereby improving the efficiency of gene editing.

LINE can be divided into stringent type and relaxed type. The part of the transcription product of stringent type LINE corresponding to 3′UTR can form a special structurally relatively conservative secondary structure, which forms a stem-loop structure at a specific position, characterized by an asymmetric loop or protrusion 4-6 bp away from the central loop. This structure promotes the binding or function of ORF2p in its corresponding species. The relaxed type generally does not form this structure, but in some cases it may form a similar structure (loop length 5-7 bp, 8-10 bp stem and a bulge 4-6 bp away from the loop), which may promote the binding and function of ORF2p. The LINEs in humans and most mammals are of the relaxed type, while the LINEs in eels (LINE UnaL2), insects (LINE R2), zebrafish (LINE ZfL2-1 and ZfL2-2), algae (L1), silkworms (LINE SART1), monocots (L1), fungi (Tad1), fish (L2) and some mammals (RTE) are of the stringent type. For the application of ORF2p corresponding to (produced by) the stringent LINE, adding the above-mentioned stem-loop structure to the ORF2p functional initiation part can increase the binding or working efficiency of the corresponding ORF2p, while for the relaxed LINE, adding the above-mentioned stem-loop structure to the ORF2p functional initiation part (such as the “UCCCGCCUGGGCCACAGAGCGAGA” sequence in the Alu element) may also increase the binding or working efficiency of the corresponding ORF2p.

The target sites on which the present invention act can be one or more; when there are multiple target sites, the corresponding genomic single strands of the primers generated by cutting the genome by ORF2p and/or ORF2p-derivative proteins corresponding to different target sites may be the same strand on the same chromosome, complementary strands on the same chromosome, or located on different chromosomes.

The sequence is designed so that the transcribed RNA sequence has two inverted repeat sequences (such as Alu elements, other SINEs or other inverted repeat sequences) or complementary sequences. The two inverted repeat sequences or complementary sequences on the RNA can combine to form a circular RNA in the portion between the two sequences. In addition, adding RNA splicing signals (sites) to the sequence when designing the sequence can also promote the formation of circular RNA from linear RNA.

Lig4, DNA-PK, and XRCC6 are specifically inhibited by sgRNA, ASO, siRNA, or specific antibodies to promote DNA homologous recombination, thereby improving the efficiency of gene editing in the present invention.

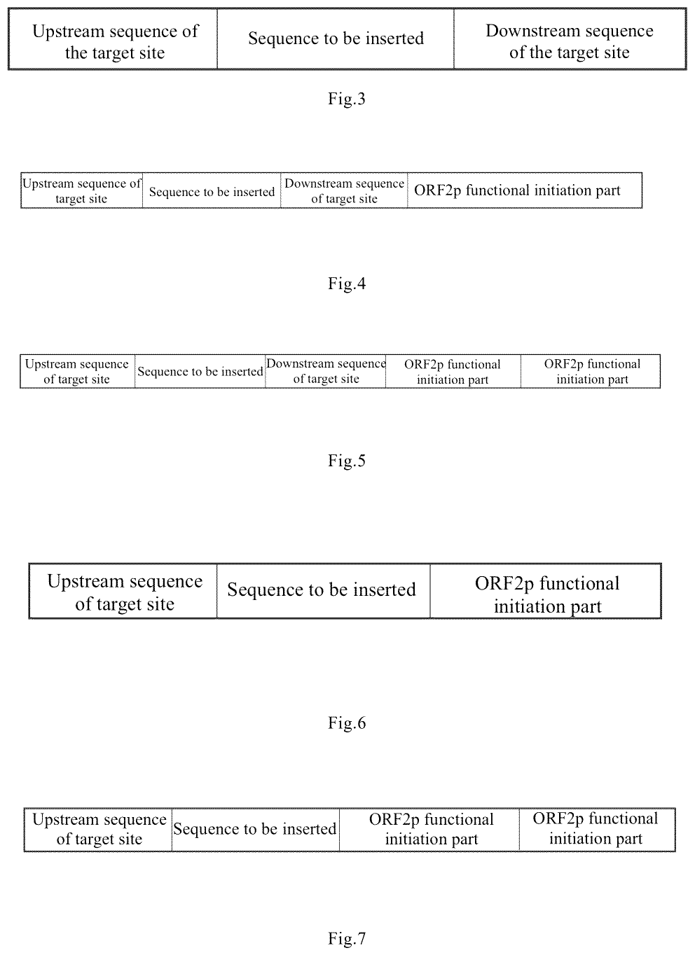

The basic structure of the RNA framework for gene editing provided by the present invention is shown in FIG. 3, which comprises an upstream sequence of target site, a sequence to be inserted and a downstream sequence of target site along the direction of 5′→3′. To better understand the further change forms of the RNA framework, some different connection forms are listed for understanding, wherein “connection” indicates direct connection, and intermediate insertion by other sequence is called “indirect connection”.

In the case of “indirectly connecting”, the intermediate inserted sequence can be any (arbitrary) sequence, any (arbitrary) sequence here is a sequence related or unrelated to the transcription of the RNA framework provided by the present invention, such as a pan-ORF1p coding sequence, a pan-ORF2p coding sequence, a long interspersed element, a short interspersed element, etc. related to the RNA framework transcription, or other coding sequences or non-coding sequences unrelated to the RNA framework transcription.

In the present invention, “between” indicates between two sequences which are still complete; and “inside” indicates that in a sequence, when a sequence is inserted inside another sequence, it indicates that another sequence is divided into two parts.

In the present invention, “interval arrangement (spaced arrangement)” refers to the arrangement of multiple different sequences when each sequence appears once or multiple times. For example, when sequence A and sequence B appear repeatedly, ABA, ABAB, ABBA, ABBABB, etc. are all different interval arrangement forms of sequence A and sequence B; when sequence A, sequence B, sequence C appear repeatedly, ABCABC, ABBCA, CCABA, etc. are all different interval arrangement forms of sequence A, sequence B, sequence C, and the same applies to more sequences.

FIG. 4 is a schematic diagram of an ORF2p functional initiation part further connected downstream of the basic structure of the RNA framework.

FIG. 5 is a schematic diagram of a structure in which multiple ORF2p functional initiation parts are further connected downstream of the basic structure of the RNA framework.

FIG. 6 is a schematic diagram of a structure in which the downstream sequence of target site is replaced with an ORF2p functional initiation part in the basic structure of the RNA framework.

FIG. 7 is a schematic diagram of the structure of replacing the downstream sequence of target site with multiple ORF2p functional initiation parts in the basic structure of the RNA framework.

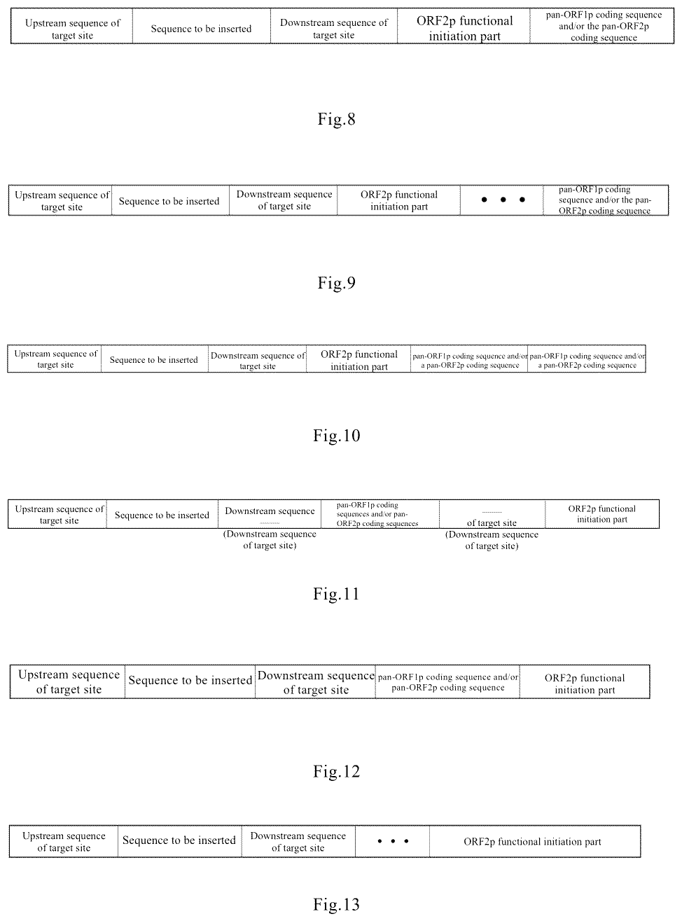

FIG. 8 is a schematic diagram of a structure in which the ORF2p functional initiation part is connected downstream of the basic structure of the RNA framework, and then a pan-ORF1p coding sequence and/or a pan-ORF2p coding sequence is connected downstream of the ORF2p functional initiation part.

FIG. 9 is a schematic diagram of a structure in which the ORF2p functional initiation part is connected downstream of the basic structure of the RNA framework, and the pan-ORF1p coding sequence and/or pan-ORF2p coding sequence is indirectly connected downstream of the ORF2p functional initiation part, represents an indirect connection.

FIG. 10 is a schematic diagram of a structure in which the ORF2p functional initiation part is connected downstream of the basic structure of the RNA framework, and then a plurality of pan-ORF1p coding sequences and/or pan-ORF2p coding sequences are connected downstream of the ORF2p functional initiation part.

FIG. 11 is a schematic diagram of a structure in which the ORF2p functional initiation part is connected downstream of the basic structure of the RNA framework, and the pan-ORF1p coding sequence and/or the pan-ORF2p coding sequence are inserted into the downstream sequence of the target site.

FIG. 12 is a schematic diagram of a structure in which the pan-ORF1p coding sequence and/or the pan-ORF2p coding sequence are connected downstream of the basic structure of the RNA framework, and the ORF2p functional initiation part is connected downstream further.

FIG. 13 is a schematic diagram of a structure in which the basic structure of the RNA framework is indirectly connected to the ORF2p functional initiation part.

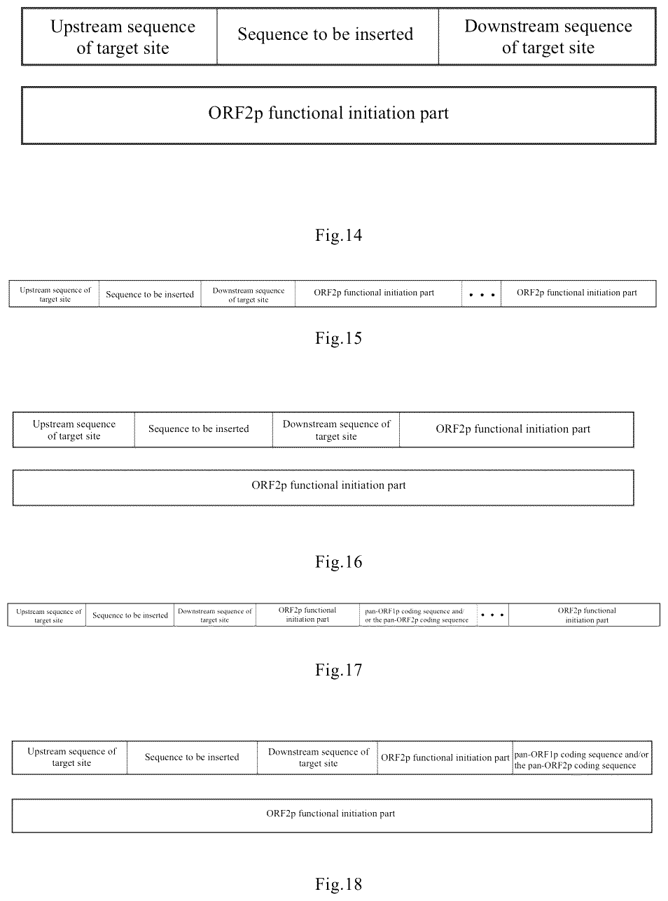

FIG. 14 is a schematic diagram of a structure in which the basic structure of the RNA framework and the ORF2p functional initiation part are located on different RNA vectors and/or RNPs.

FIG. 15 is a schematic diagram of a structure in which two ORF2p functional initiation parts are connected downstream of the basic structure of the RNA framework and the two ORF2p functional initiation parts are indirectly connected.

FIG. 16 is a schematic diagram of a structure in which the ORF2p functional initiation part is connected downstream of the basic structure of the RNA framework, and the ORF2p functional initiation part is located on different RNA vectors and/or RNPs with another ORF2p functional initiation part.

FIG. 17 is a schematic diagram of a structure in which the ORF2p functional initiation part is connected downstream of the basic structure of the RNA framework, and the pan-ORF1p coding sequence and/or the pan-ORF2p coding sequence are directly connected downstream, and the pan-ORF1p coding sequence and/or the pan-ORF2p coding sequence are indirectly connected to the ORF2p functional initiation part downstream thereof.

FIG. 18 is a schematic diagram of a structure in which the ORF2p functional initiation part is connected downstream of the basic structure of the RNA framework, and the pan-ORF1p coding sequence and/or the pan-ORF2p coding sequence are connected downstream, and the ORF2p functional initiation part is located on different RNA vectors and/or RNPs with another ORF2p functional initiation part.

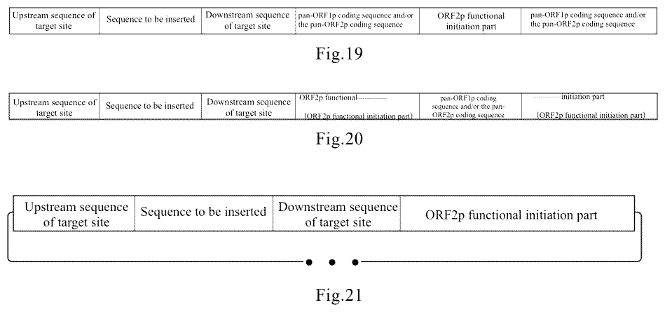

FIG. 19 is a schematic diagram of a structure in which interval-arranged pan-ORF1p coding sequence and/or pan-ORF2p coding sequence and the ORF2p functional initiation part are connected downstream of the basic structure of the RNA framework.

FIG. 20 is a schematic diagram of a structure in which the ORF2p functional initiation part is connected downstream of the basic structure of the RNA framework, and the ORF2p functional initiation part is internally connected with a pan-ORF1p coding sequence and/or a pan-ORF2p coding sequence.

FIG. 21 is a schematic diagram of a structure in which the ORF2p functional initiation part is connected downstream of the basic structure of the RNA framework and a circular RNA form is formed.

In the following embodiments, since the materials used are human cells, the short interspersed elements used are Alu Ya5 in the primate-specific short interspersed element Alu element. The complete sequence of the Alu Ya5 element is shown in Seq ID No. 1, and the partial Alu Ya5 sequence is shown in Seq ID No. 2. When the materials used are other species, the short interspersed elements can be replaced with short interspersed elements of the corresponding species to increase the efficiency of gene editing.

### Material

1. The pBudORF1-CH plasmid is purchased from Addgene company, plasmid number: 51290; the pBudORF2-CH plasmid is purchased from Addgene company, plasmid number: 51289; the pBS-L1PA1-CH-mneo plasmid is purchased from Addgene company, plasmid number: 51288; the pBudORF1-CH plasmid, pBudORF2-CH plasmid, and pBS-L1PA1-CH-mneo plasmid were entrusted to Beijing Hesheng Biotechnology Co., Ltd. for amplification.

2. CD293 culture medium was purchased from Thermo Fisher Scientific, product number: 11913019.

3. PEI transfection reagent was purchased from Serochem company, product number: Prime-AQ100-100ML.

4. SMS 293-SUPI was purchased from Beijing Sino Biological Inc., with product number: M293-SUPI-100.

5. Potassium acetate was purchased from Sigma Aldrich company, product number: P1190.

6. Tris HCl (pH 7.5) was purchased from Shanghai Shangbao Biotechnology Co., Ltd., product number: T16588.

7. Glycerol was purchased from Sigma Aldrich, product number: G5516.

8. Triton X-100 was purchased from Sigma Aldrich, product number: T8787.

9. PMSF protease inhibitor was purchased from Thermo Fisher Scientific, product number: 36978.

10. Ni affinity chromatography column (HISTRAP HP) was purchased from Cytiva.

11. Imidazole purchased from Sigma Aldrich company, product number: 15513.

12. Rabbit anti-HIS was purchased from Sigma Aldrich company, product number: SAB1306082.

13. BSA was purchased from Sigma Aldrich, product number: A1933.

14. Anti rabbit IgG—alkaline phosphatase goat antibody purchased from Sigma Aldrich company, product number: A3687.

15. pcDNA™ 3.1(+) Purchased from Invitrogen company, product number: V79020.

16. NheI was purchased from Thermo Fisher company, and the formula for 10×enzyme digestion buffer was: 330 mM Tris acetate, 100 mM magnesium acetate, 660 mM potassium acetate, 1 mg/mL BSA.

17. T4 DNA ligase and its 10×buffer purchased from Promega.

18. MEGAscript™ T7 Transcription Kit was purchased from Thermo Fisher Scientific, product number: AM1333.

19. Opti-MEM™ I culture medium was purchased from Thermo Fisher Scientific, product number: A4124802.

20. The RNase inhibitor was purchased from Thermo Fisher Scientific with product number: AM2694.

21. RNAiMAX transfection reagent was purchased from Thermo Fisher Scientific, product number: 13778030.

22. pcDNA3.1(+)eGFP was purchased from Addgene company, product number: 129020.

23. KOD One™ PCR Master Mix was purchased from Dongyangfang (Shanghai) Biotechnology Co., Ltd., product number: KMM-201S.

24. One step rapid cloning kit (Hieff Clone® Plus One Step Cloning Kit) was purchased from Yisheng Biotechnology (Shanghai) Co., Ltd., product number: 10911ES20.

25. The complete culture medium is made of 90% DMEM medium and 10% fetal bovine serum. The DMEM medium is purchased from Thermo Fisher Scientific with product number 11965092, and the fetal bovine serum is purchased from Thermo Fisher Scientific with product number 10100147.

26. The Entranster-H4000 transfection reagent was purchased from Beijing Engreen Biotechnology Co., Ltd.

27. The blood/cell/tissue genomic DNA extraction kit was purchased from Tiangen Biochemical Technology (Beijing) Co., Ltd., product catalog number: DP304.

28. The MEGAscript™ SP6 transcription kit was purchased from Thermo Fisher Scientific, product number: AM1330.

29. SuperReal PreMix Plus (SYBR Green) was purchased from Tiangen Biochemical Technology (Beijing) Co., Ltd., product catalog number: FP205.

30. The chemical synthesis of primers and sequences was completed by Boshang Biotechnology (Shanghai) Co., Ltd. or ABIOCENTER (Jiangsu) Biotechnology Co., Ltd.

### Embodiment 1 Preparation of ORF1p and ORF2p

### 1. Preparation of Human ORF1p in LINE-1 (LRE1)

The commercial plasmid pBudORF1-CH has been used for expression of ORF1p (abbreviated as hLRE1-ORF1p) in human LINE-1 (LRE1). Therefore, hLRE1-ORF1p can be obtained by directly using the pBudORF1-CH plasmid for expression.

1) Transfection of pBudORF1-CH Plasmid and Expression

Culture and passage HEK293 cells in CD293 medium, and then transfect the pBudORF1-CH plasmid into HEK293 cells according to the PEI transfection reagent instructions. Add SMS 293-SUPI solution according to the instructions on the 1st, 3rd, and 5th day after transfection. HEK293 cells were cultured in shake flasks under the following conditions: 5% CO2, temperature of 37° C., and shaking speed of 175 rpm. Reactor cultivation conditions: pH 7.2, temperature 37° C., stirring speed 150 rpm, dissolved oxygen 40%. Incubate HEK293 cells in a shaker incubator, and after transfection for 7 days, centrifuge at 3000 g for 5 minutes to recover the cells. The added SMS 293-SUPI can promote cell survival and increase protein production.

2) Extraction of hLRE1-ORF1p

Prepare cell lysis buffer (100 mM potassium acetate, 50 mM Tris HCl (pH7.5), 5% glycerol, 0.3% Triton X-100) and pre cool. Before use, add protease inhibitor PMSF to maintain a concentration of 1 mM in the cell lysis buffer. Add cell lysis buffer to the cells for lysis, and add 40 ml of cell lysis buffer to each liter of cells for lysis (RIPA lysis buffer or other types of cell lysis buffer can also be used during the lysis process). Afterwards, use the pipette tip to blow and thoroughly lyse the cells, avoiding foaming and swirling. Afterwards, use a glass homogenizer to continue processing the cells, followed by 3 cycles of ultrasound (working for 15 seconds with an interval of 15 seconds). Centrifuge 30000 g at 4° C. for 25 minutes, retain the supernatant, and filter with a 0.22 um filter membrane. Take the filtered sample and purify the protein using a Ni affinity chromatography column (HISTRIP HP).

The protein purification steps are as follows:
1. Rinse the Ni affinity chromatography column with deionized water that is 5 times the volume of the column;
2. Prepare PBS buffer with pH 7.4 and equilibrate the Ni affinity chromatography column with 5 to 10 times the column volume of PBS buffer;
3. Flow the prepared supernatant through the Ni affinity chromatography column at a speed of 0.5 ml/min;
4. Use the configured buffer to rebalance the Ni affinity chromatography column;
5. Prepare solutions containing 20 mM imidazole, 50 mM imidazole, 100 mM imidazole, 250 mM imidazole, and 500 mM imidazole using 0.5M NaCl;
6. Wash the protein samples on the nickel column with solutions containing 20 mM imidazole, 50 mM imidazole, 100 mM imidazole, 250 mM imidazole, and 500 mM imidazole, and collect the corresponding elution samples.

Dialyze the collected elution samples overnight at 4° C. and use the prepared PBS solution as a buffer. Finally, the dialyzed sample was concentrated by ultrafiltration (using appropriate ultrafiltration tubes) and subjected to SDS-PAGE (primary antibody used: rabbit anti his 1:1500 (5% Milk+0.1% BSA); Secondary antibody: Goat anti rabbit IgG alkaline phosphatase 1:6000 (5% Milk) was used to detect the target protein obtained, confirm the protein concentration, and then hLRE1-ORF1p was extracted, purified, and freeze-dried.

### 2. Preparation of ORF2p in Human LINE-1 (LRE1)

The commercial plasmid pBudORF2-CH has been used for expression of ORF2p (abbreviated as hLRE1-ORF2p) in human LINE-1 (LRE1). Therefore, hLRE1-ORF2p can be obtained by directly using the pBudORF2-CH plasmid for expression.

Replace the plasmid pBudORF1-CH expressing hLRE1-ORF1p with the plasmid pBudORF2-CH expressing hLRE1-ORF2p, and prepare purified lyophilized hLRE1-ORF2p according to the preparation method of hLRE1-ORF1p.

### 3. Preparation of ORF1p in Human LINE-1 (LRE2)

There is no commercially available plasmid for direct expression of ORF1p (hLRE2-ORF1p) in human LINE-1 (LRE2), so an expression plasmid was constructed for expression.

The DNA sequence encoding ORF1p in human LINE-1 (LRE2) is shown in Seq ID No.3: ATGGGGAAAAAACAGAACAGAAAAACTGGAAACTCTAAAACGCAGAGCG CCTCTCCTCCTCCAAAGGAACGCAGTTCCTCACCAGCAACAGAACAAAGCTGGA TGGAGAATGATTTTGACGAGCTGAGAGAAGAAGGCTTCAGACGATCAAATTACT CTGAGCTACGGGAGGACATTCAAACCAAAGGCAAAGAAGTTGAAAACTTTGAAA AAAATTTAGAAGAATGTATAACTAGAATAACCAATACAGAGAAGTGCTTAAAGG AGCTGATGGAGCTGAAAACCAAGGCTCGAGAACTACGTGAAGAATGCAGAAGC CTCAGGAGCCGATGCGATCAACTGGAAGAAAGGGTATCAGCAATGGAAGATGA AATGAATGAAATGAAGCGAGAAGGGAAGTTTAGAGAAAAAAGAATAAAAAGAA ATGAGCAAAGCCTCCAAGAAATATGGGACTATGTGAAAAGACCAAATCTACGTC TGATTGGTGTACCTGAAAGTGATGTGGAGAATGGAACCAAGTTGGAAAACACTC TGCAGGATATTATCCAGGAGAACTTCCCCAATCTAGCAAGGCAGGCCAACGTTC AGATTCAGGAAATACAGAGAACGCCACAAAGATACTCCTCGAGAAGAGCAACTC CAAGACACATAATTGTCAGATTCACCAAAGTTGAAATGAAGGAAAAAATGTTAA GGGCAGCCAGAGAGAAAGGTCGGGTTACCCTCAAAGGGAAGCCTATCAGACTAA CAGCAGATCTCTCGGCAGAAACCCTACAAGCCAGAAGAGAGTGGGGGCCAATAT TCAACATTCTTAAAGAAAAGAATTTTCAACCCAGAATTTCATTTCCAGCCAAACT AAGCTTCATAAGTGAAGGAGAAAGAAAATACTTTACAGACAAGCAAATGCTGAG AGATTTTGTCACCACCAGGCCTACCCTAAAAGAGCTCCTGAAGGAAGCACTAAA CATGGAAAGGAACAACCGGTACCAGCCGCTGCAAAATCATGCCAAAATGTAA.

Remove the “taa” at the end of the DNA sequence of ORF1p in human LINE-1 (LRE2), then add the encoding sequences of Myc tag and His tag (wavy lines), then add “tga” (bold in italics), and finally add NheI restriction enzyme cutting site and protective bases (CTAGCTAGCTAG) at both ends of the sequence. The modified nucleotide sequence is shown in Seq ID No.4:

CTAGCTAGCTAGATGGGGAAAAAACAGAACAGAAAAACTGGAAACTCTAA
AACGCAGAGCGCCTCTCCTCCTCCAAAGGAACGCAGTTCCTCACCAGCAACAGA
ACAAAGCTGGATGGAGAATGATTTTGACGAGCTGAGAGAAGAAGGCTTCAGACG
ATCAAATTACTCTGAGCTACGGGAGGACATTCAAACCAAAGGCAAAGAAGTTGA
AAACTTTGAAAAAAATTTAGAAGAATGTATAACTAGAATAACCAATACAGAGAA
GTGCTTAAAGGAGCTGATGGAGCTGAAAACCAAGGCTCGAGAACTACGTGAAGA
ATGCAGAAGCCTCAGGAGCCGATGCGATCAACTGGAAGAAAGGGTATCAGCAAT
GGAAGATGAAATGAATGAAATGAAGCGAGAAGGGAAGTTTAGAGAAAAAAGAA
TAAAAAGAAATGAGCAAAGCCTCCAAGAAATATGGGACTATGTGAAAAGACCA
AATCTACGTCTGATTGGTGTACCTGAAAGTGATGTGGAGAATGGAACCAAGTTG
GAAAACACTCTGCAGGATATTATCCAGGAGAACTTCCCCAATCTAGCAAGGCAG
GCCAACGTTCAGATTCAGGAAATACAGAGAACGCCACAAAGATACTCCTCGAGA
AGAGCAACTCCAAGACACATAATTGTCAGATTCACCAAAGTTGAAATGAAGGAA
AAAATGTTAAGGGCAGCCAGAGAGAAAGGTCGGGTTACCCTCAAAGGGAAGCCT
ATCAGACTAACAGCAGATCTCTCGGCAGAAACCCTACAAGCCAGAAGAGAGTGG
GGGCCAATATTCAACATTCTTAAAGAAAAGAATTTTCAACCCAGAATTTCATTTC
CAGCCAAACTAAGCTTCATAAGTGAAGGAGAAAGAAAATACTTTACAGACAAGC
AAATGCTGAGAGATTTTGTCACCACCAGGCCTACCCTAAAAGAGCTCCTGAAGG
AAGCACTAAACATGGAAAGGAACAACCGGTACCAGCCGCTGCAAAATCATGCCA

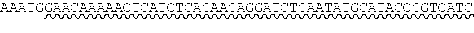

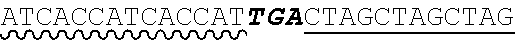

The nucleotide of Seq ID No.4 was obtained by chemical synthesis, and then NheI enzyme digestion was performed to obtain the sequence to be inserted. Simultaneously targeting pcDNA™3.1(+) plasmids were also subjected to NheI enzyme digestion to obtain linear plasmids; Then, the electrophoresis and recovery of the sequence to be inserted and the linear plasmid were conducted.

The specific enzyme digestion reaction system is shown in Table 1:

TABLE 1
Enzyme digestion reaction system

| Component | Addition Amount | |
|---|---|---|
| Nucleotide of Seq ID No.4 or | 1 | μg |
| pcDNA ™3.1(+) | | |
| NheI | 1 | μL |
| 10 × restriction enzyme buffer | 5 | μL |
| ddH2O | Make up to 50 μL | |
| Total | 50 | μL |

The reaction conditions were as follows: incubation at 37° C. for 3 h, followed by heating to 80° C. for 10 min to inactivate the endonuclease.

Connect the recovered sequence to the linear plasmid after enzyme digestion, electrophoresis, and recovery to obtain the pcDNA™3.1(+)-hLRE2-ORF1p for expressing ORF1p in human LINE-1 (LRE2).

The specific connection reaction system is shown in Table 2:

TABLE 2
ligation reaction system

| Component | Addition Amount | |
|---|---|---|
| the sequence to be inserted | 20 | ng |
| pcDNA ™3.1(+) | 100 | ng |
| T4 DNA ligase | 1 Weiss unit | |
| 10 × ligation buffer | 1 | μL |

| | |
|---|---|
| Nuclease-free water | Make up to 10 μL |

The ligation reaction conditions were as follows: incubate at 16° C. for 16 hours, then heat up to 70° C. and incubate for 10 minutes to inactivate the ligase.

Preparation of ORF1 protein (hLRE2-ORF1p) in human LINE-1 (LRE2): Following the preparation method of hLRE1-ORF1p, replace the plasmid pBudORF1-CH with pcDNA™3.1(+)-hLRE2-ORF1p, and pcDNA™3.1(+)-hLRE2-ORF1p was transfected and expressed, and the obtained was purified and freeze-dried to obtain hLRE2-ORF1p.

### 4. Preparation of ORF2p in Human LINE-1 (LRE2)

There is no commercially available plasmid for direct expression of ORF2p (hLRE2-ORF2p) in human LINE-1 (LRE2), so an expression plasmid was constructed for expression.

The coding nucleotide sequence of ORF2p in human LINE-1 (LRE2) is shown in Seq ID No.5. hLRE2-ORF2p was prepared, expressed, purified, and freeze-dried according to the preparation method of ORF1p in human LINE-1 (LRE2).

### 5. Preparation of Mouse ORF1p

The DNA sequence of the coding sequence of mouse ORF1p is shown in Seq ID No.6. Following the preparation method of ORF1p in human LINE-1 (LRE2), the expression and purification of lyophilized mouse ORF1p were prepared and named mORF1p.

### 6. Preparation of Mouse ORF2p

The DNA sequence of the coding sequence of mouse ORF2p is shown in Seq ID No.7. Following the preparation method of ORF1p in human LINE-1 (LRE2), the expression and purification of lyophilized mouse ORF2p were prepared and named mORF2p.

### Embodiment 2 Inspection of the Effect of Gene Editing by Introducing RNA Produced by In Vitro Transcription into a Target System with/without Binding of ORF1p and/or ORF2p Outside the Target System

The Lman1 gene is the pathogenic gene for the combined deficiency of factor V and VIII (F5F8D), and its mutation can lead to a decrease in human FV and FVIII levels, and patients may exhibit spontaneous bleeding symptoms.

Select a 405 bp sequence from the Lman1 gene in the human genome, as shown in Seq ID No.8:

GGGTAGAGATTCACTGCCTTAGTCTCATGTAGTCTCGTGTAGTCTTTTG
AGTAAATAACATAAAGTATCTCAAGACTTTTTCATAACTTGATATTATT
TTAGTCTTCCTGAATTTTTAAATATTGAAAAGCTGAGTGTCTTGTCTGT
TTT*CCTCCCCCTTACACTATAGTGACGGGGCTAGTCAAGCTTTGGCAA
GTTGCCAGAGGGACTTCCGCAACAAACCCTATCCTGTCCGAGCAAAGAT
TACCTATTACCAGAACACACTGACAGTAAGTAACATCTATTTAGAGAGA
ATCAAATAAACAATGTTACAGTATCACTTTTCATTTTGAATTTTTGATA
GAAATTAAATGCACTTAAATTTGGATATGCTTACATACTCTTCATTGTT
ACTCTAAGAGAACG;

wherein * was a selected insertion site (target site). The sequence upstream of the insertion site in the Lman1 gene (upstream sequence of target site) was before the insertion site, and the sequence downstream of the insertion site in the Lman1 gene (downstream sequence of target site) was after the insertion site. An exogenous sequence was added at an insertion site as a sequence to be inserted, its sequence is shown in Seq ID No.9:

AGGTGCCTGCACATACTGCATGTGAGAGTCTGGAGACGCCAGACTGTTC
TGAGTCCTGACCTGCTCAGGGGTGAGGTCCCTCTGAGCCTGAGCAAGCA
TTTCGTAGCCAACCATGAATTTCCGGACAGTGGCAGAGCGCAGGAGCGG
AGG;

The sequence generated after insertion is shown in Seq ID No.10:

GGGTAGAGATTCACTGCCTTAGTCTCATGTAGTCTCGTGTAGTCTTTTG
AGTAAATAACATAAAGTATCTCAAGACTTTTTCATAACTTGATATTATT
TTAGTCTTCCTGAATTTTTAAATATTGAAAAGCTGAGTGTCTTGTCTGT
TTTAGGTGCCTGCACATACTGCATGTGAGAGTCTGGAGACGCCAGACTG
TTCTGAGTCCTGACCTGCTCAGGGGTGAGGTCCCTCTGAGCCTGAGCAA
GCATTTCGTAGCCAACCATGAATTTCCGGACAGTGGCAGAGCGCAGGAG
CGGAGGCCTCCCCCTTACACTATAGTGACGGGGCTAGTCAAGCTTTGGC
AAGTTGCCAGAGGGACTTCCGCAACAAACCCTATCCTGTCCGAGCAAAG
ATTACCTATTACCAGAACACACTGACAGTAAGTAACATCTATTTAGAGA
GAATCAAATAAACAATGTTACAGTATCACTTTTCATTTTGAATTTTTGA
TAGAAATTAAATGCACTTAAATTTGGATATGCTTACATACTCTTCATTG
TTACTCTAAGAGAACG;

Wherein the underline represents the sequence to be inserted as shown in Seq ID No.9.

Add the T7 promoter sequence shown in Seq ID No.11 upstream of the sequence shown in Seq ID No.10 (Seq ID No.11: TAATACGACTCACTATA), and add a partial Alu sequence shown in Seq ID No.2 downstream. After addition, the sequence is shown in Seq ID No.12:

TAATACGACTCACTATAGGGTAGAGATTCACTGCCTTAGTCTCATGTAGTCT
CGTGTAGTCTTTTGAGTAAATAACATAAAGTATCTCAAGACTTTTTCATAACTTG
ATATTATTTTAGTCTTCCTGAATTTTTAAATATTGAAAAGCTGAGTGTCTTGTCTG
TTTTAGGTGCCTGCACATACTGCATGTGAGAGTCTGGAGACGCCAGACTGTTCTG
AGTCCTGACCTGCTCAGGGGTGAGGTCCCTCTGAGCCTGAGCAAGCATTTCGTAG
CCAACCATGAATTTCCGGACAGTGGCAGAGCGCAGGAGCGGAGGCCTCCCCCTT
ACACTATAGTGACGGGGCTAGTCAAGCTTTGGCAAGTTGCCAGAGGGACTTCCG
CAACAAACCCTATCCTGTCCGAGCAAAGATTACCTATTACCAGAACACACTGAC
AGTAAGTAACATCTATTTAGAGAGAATCAAATAAACAATGTTACAGTATCACTTT
TCATTTTGAATTTTTGATAGAAATTAAATGCACTTAAATTTGGATATGCTTACATA

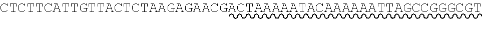

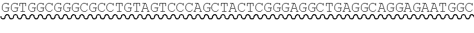

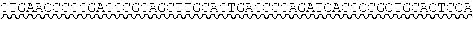

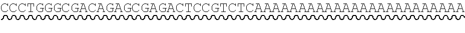

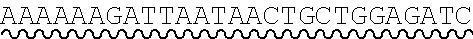

Wherein the underlined part represents the sequence to be inserted as shown in Seq ID No.9, the italicized bold part represents the T7 promoter sequence as shown in Seq ID No.11, and the wavy line represents the partial Alu sequence as shown in Seq ID No.2. The sequence was obtained through chemical synthesis and named precursor DNA of RNA+partial Alu.

Add the T7 promoter sequence shown in Seq ID No.11 upstream of the sequence shown in Seq ID No.10. The sequence after addition is shown in Seq ID No.13:

TAATACGACTCACTATAGGGTAGAGATTCACTGCCTTAGTCTCATGTAG
TCTCGTGTAGTCTTTTGAGTAAATAACATAAAGTATCTCAAGACTTTTT
CATAACTTGATATTATTTTAGTCTTCCTGAATTTTTAAATATTGAAAAG
CTGAGTGTCTTGTCTGTTTTAGGTGCCTGCACATACTGCATGTGAGAGT
CTGGAGACGCCAGACTGTTCTGAGTCCTGACCTGCTCAGGGGTGAGGTC
CCTCTGAGCCTGAGCAAGCATTTCGTAGCCAACCATGAATTTCCGGACA
GTGGCAGAGCGCAGGAGCGGAGGCCTCCCCCTTACACTATAGTGACGGG
GCTAGTCAAGCTTTGGCAAGTTGCCAGAGGGACTTCCGCAACAAACCCT
ATCCTGTCCGAGCAAAGATTACCTATTACCAGAACACACTGACAGTAAG
TAACATCTATTTAGAGAGAATCAAATAAACAATGTTACAGTATCACTTT
TCATTTTGAATTTTTGATAGAAATTAAATGCACTTAAATTTGGATATGC
TTACATACTCTTCATTGTTACTCTAAGAGAACG,

Wherein the underline represents the sequence to be inserted as shown in Seq ID No.9, and the italicized bold represents the T7 promoter sequence as shown in Seq ID No.11. This sequence was obtained through chemical synthesis and named as the precursor DNA of RNA.

According to the instructions of the MEGAscript™ T7 Transcription Kit, the linear precursor DNA of RNA+partial Alu or precursor DNA of RNA were transcribed to obtain corresponding RNA. Then, the residual DNA was degraded by DNase from the kit and resuspended in RNase free water. The RNA concentration was measured by UV spectrophotometer, and the concentration of RNA+partial Alu solution or RNA solution was further configured to 100 ng/μL by adding RNase free water.

The RNA+partial Alu obtained from the above transcription belongs to the RNA framework structure in FIG. 4, where the ORF2p functional initiating (initiation) part is partial short interspersed element (SINE). The RNA transcribed above belongs to the RNA framework structure shown in FIG. 3.

Resuspend hLRE1-ORF1p and hLRE1-ORF2p prepared in embodiment 1 with Opti MEM solution pre added with 1 U/μL RNA enzyme inhibitor, respectively, into 500 ng/μL of hLRE1-ORF1p solution or hLRE1-ORF2p solution.

Prepare RNP of RNA or RNA+partial Alu bound with ORF1p and/or ORF2p.

Wherein the RNA+partial Alu combined with hLRE1-ORF1p and hLRE1-ORF2p are called RNA+partial Alu+hLRE1-ORF1p and RNA+partial Alu+hLRE1-ORF2p, respectively. The reaction system is shown in Table 3:

TABLE 3
Reaction System

| Component | Addition Amount |
|---|---|
| RNA + partial Alu solution | 21.0 μL |
| hLRE1-ORF1p solution or | 3.0 μL (hLRE1-ORF1p |
| hLRE1-ORF2p solution | solution) or |
| | 5.5 μL (hLRE1-ORF2p |
| | solution) |
| Opti-MEM ™ I medium | Make up to 75 μL |

Due to the different binding amounts of hL1-ORF1p solution and hL1-ORF2p solution with RNA, the amount added also varies.

After gently mixing each component, the reaction system was incubated at room temperature (25° C.) for 10 minutes to obtain RNA+partial Alu+hLRE1-ORF1p and RNA+partial Alu+hLRE1-ORF2p, respectively.

The preparation of RNA+hLRE1-ORF1p+hLRE1-ORF2p and RNA+partial Alu+hLRE1-ORF1p+hLRE1-ORF2p is carried out as follows:

Configure the reaction system as shown in Table 4:

TABLE 4
Reaction System

| Component | Addition Amount | |
|---|---|---|
| RNA solution or RNA + | 21.0 | μL |
| partial Alu solution | | |
| hLRE1-ORF2p solution | 5.5 | μL |

| | |
|---|---|
| Opti-MEM ™ I medium | Make up to 50 μL |

Gently mix the components and incubate the reaction system at room temperature (25° C.) for 10 minutes to obtain RNA+hLRE1-ORF2p solution and RNA+partial Alu+hLRE1-ORF2p solution, respectively.

Then, mix the RNA+hLRE1-ORF2p solution and RNA+partial Alu+hLRE1-ORF2p solution according to the system shown in Table 5.

TABLE 5
Reaction System

| Component | Addition Amount | |
|---|---|---|
| RNA + hLRE1-ORF2p | 50.0 | μL |
| solution or RNA + partial | | |
| Alu + hLRE1-ORF2p | | |
| solution | | |
| hLRE1-ORF1p solution | 3.0 | μL |

| | |
|---|---|
| Opti-MEM ™ I medium | Make up to 75 μL |

After gently mixing each component, the reaction system was incubated at room temperature (25° C.) for 10 minutes to obtain RNA+hLRE1-ORF1p+hLRE1-ORF2p solution and RNA+partial Alu+hLRE1-ORF1p+hLRE1-ORF2p solution, respectively.

Prepare hLRE1-ORF1p+hLRE1-ORF2p solution containing only hLRE1-ORF1p and hLRE1-ORF2p without RNA or RNA+partial Alu as a negative control. The reaction system is shown in Table 6.

TABLE 6
Reaction System

| Component | Addition Amount |
|---|---|
| hLRE1-ORF1p solution | 3.0 μL |
| hLRE1-ORF2p solution | 5.5 μL |
| Opti-MEM ™ I medium | Make up to 75 μL |

Gently mix each component and incubate the reaction system at room temperature (25° C.) for 10 minutes to obtain a solution of hLRE1-ORF1p+hLRE1-ORF2p.

Configure the transfection solution, as shown in Table 7:

TABLE 7
Transfection Solution System

| Component | Addition Amount |
|---|---|
| RNAiMAX transfection | 7.0 μL |
| reagent | |
| Opti-MEM ™ I medium | Make up to 75 μL |

Add the transfection reagent to hLRE1-ORF1p+hLRE1-ORF2p solution, RNA+partial Alu+hLRE1-ORF1p solution, RNA+partial Alu+hLRE1-ORF2p solution, RNA+hLRE1-ORF1p+hLRE1-ORF2p solution, and RNA+partial Alu+hLRE1-ORF1p+hLRE1-ORF2p solution in equal volume ratios. Mix gently and incubate at room temperature (25° C.) for 20 minutes to obtain the corresponding transfection solution. The liposomes contained in the transfection solution will form complexes with RNA, RNP, or proteins in the solution, namely hLRE1-ORF1p+hLRE1-ORF2p-liposome complex, RNA+partial Alu+hLRE1-ORF1p-liposome complex, RNA+partial Alu+hLRE1-ORF2p-liposome complex, RNA+hLRE1-ORF1p+hLRE1-ORF2p-liposome complex, RNA+partial Alu+hLRE1-ORF1p+hLRE1-ORF2p-liposome complex.

Prepare liposome complex transfection solution of RNA+partial Alu without hLRE1-ORF1p or hLRE1-ORF2p, and the reaction system is shown in Table 8:

TABLE 8
Reaction System

| Component | Addition Amount |
|---|---|
| RNA + partial Alu solution | 21.0 μL |
| Opti-MEM ™ I medium | Make up to 75 μL |

After gently mixing each component, the reaction system was incubated at room temperature (25° C.) for 10 minutes. Then, transfection solution shown in Table 7 was added in equal volume ratios. The mixture was gently mixed and incubated at room temperature (25° C.) for 20 minutes to obtain RNA+partial Alu-liposome complex.

Construction of Direct Transfection Plasmid pcDNA3.1(+)eGFP+RNA+Partial Alu

After connecting the sequence shown in Seq ID No.2 after the sequence shown in Seq ID No.10, the sequence shown in Seq ID No.14 was obtained:

GGGTAGAGATTCACTGCCTTAGTCTCATGTAGTCTCGTGTAGTCTTTTGAGT
AAATAACATAAAGTATCTCAAGACTTTTTCATAACTTGATATTATTTTAGTCTTCC
TGAATTTTTAAATATTGAAAAGCTGAGTGTCTTGTCTGTTTTAGGTGCCTGCACAT
ACTGCATGTGAGAGTCTGGAGACGCCAGACTGTTCTGAGTCCTGACCTGCTCAGG
GGTGAGGTCCCTCTGAGCCTGAGCAAGCATTTCGTAGCCAACCATGAATTTCCGG
ACAGTGGCAGAGCGCAGGAGCGGAGGCCTCCCCCTTACACTATAGTGACGGGGC
TAGTCAAGCTTTGGCAAGTTGCCAGAGGGACTTCCGCAACAAACCCTATCCTGTC
CGAGCAAAGATTACCTATTACCAGAACACACTGACAGTAAGTAACATCTATTTA
GAGAGAATCAAATAAACAATGTTACAGTATCACTTTTCATTTTGAATTTTTGATA
GAAATTAAATGCACTTAAATTTGGATATGCTTACATACTCTTCATTGTTACTCTAA

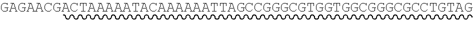

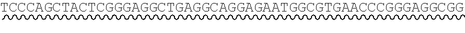

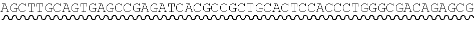

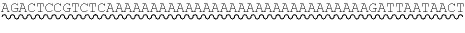

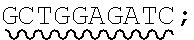

Wherein the underlined represents the sequence to be inserted as shown in Seq ID No.9; The wavy line represents a partial Alu sequence as shown in Seq ID No.2, the sequence was named RNA+partial Alu, obtained through chemical synthesis.

The sequence shown in Seq ID No.14 was chemically synthesized and constructed into the pcDNA3.1(+)eGFP vector through homologous recombination linkage, so that the sequence is directly connected downstream of the CMV promoter in the vector, and there are no other sequences between the sequence and the CMV promoter. The plasmid obtained was named pcDNA3.1 (+) eGFP+RNA+partial Alu.

The specific steps are:

1. Design primers to amplify the sequence shown in Seq ID No.14, where the forward primer sequence is as shown in Seq ID No.15: 5′-CTATATAAGCAGAGCTGGGTAGAGATTCACTG-3′, and the reverse primer sequence is as shown in Seq ID No.16: 5′-CTCTAGTTAGCCAGAGGATCTCCAGCAGTTAT-3′. Perform PCR amplification on the sequence shown in Seq ID No.14, and the reaction system is shown in Table 9:

TABLE 9
Reaction System

| Component | Addition Amount | |
|---|---|---|
| 2 × KOD PCR Master mix | 25 | μL |
| forward primer (10 μM) | 1 | μL |
| reverse primer (10 μM) | 1 | μL |
| RNA + partial Alu (10 pg- | ~1 | μL |
| 200 ng) | | |
| ddH₂O | ~22 | μL |

The amplification conditions are: 94° C. for 2 minutes; Perform 40 cycles at 98° C. for 10 seconds, 60° C. for 10 seconds, and 68° C. for 2 seconds; 68° C. for 5 minutes.

The amplification product was obtained by gel electrophoresis, extraction and purification with conventional methods. The two sides of the synthesized sequence in the amplification product were added with the sequence homologous to pcDNA3.1(+)eGFP vector.

2. Design PCR primers to amplify the pcDNA3.1(+)eGFP vector, with a forward primer as shown in Seq ID No.17: 5′-AATAACTGCTGGAGATCCTCTGGCTAACTAGAG-3′ and a reverse primer sequence as shown in Seq ID No.18: 5′-CAGTGAATCTCTACCCAGCTCTGCTTATATAG-3′. Perform PCR amplification on the pcDNA3.1(+)eGFP vector, and the reaction system is shown in Table 10:

TABLE 10
Reaction System

| Component | Addition Amount | |
|---|---|---|
| 2 × KOD PCR Master mix | 25 | μL |
| forward primer (10 μM) | 1 | μL |
| reverse primer (10 μM) | 1 | μL |
| pcDNA3.1(+)eGFP | ~1 | μL |
| plasmid template (10 pg- | | |
| 200 ng) | | |
| ddH₂O | ~22 | μL |

The amplification conditions are: 94° C. for 2 minutes; Perform 40 cycles at 98° C. for 10 seconds, 60° C. for 10 seconds, and 68° C. for 6 seconds; 68° C. for 5 minutes.

The pcDNA3.1(+)eGFP plasmid vector was obtained by gel electrophoresis, extraction and purification with conventional methods. The two ends of the plasmid vector had homologous sequences with the synthesized sequence.

3. Use a one-step rapid cloning kit to connect the amplified product to the amplified pcDNA3.1(+)eGFP vector. Follow the instructions in the kit for specific steps, and the reaction system is shown in Table 11:

TABLE 11
Connection reaction system

| Component | Addition Amount | |
|---|---|---|
| Amplified fragment | 150-200 | ng |
| Amplified vector | 50 | ng |
| 2 × Hieff Clone Enzyme | 10 | μL |
| Premix | | |

| | |
|---|---|
| H₂O | Up to 20 μL |

4. Transfect the recombinant product into competent cells, pick the colonies on plate and sequence. After sequencing, extract the plasmid to obtain pcDNA3.1(+)eGFP-RNA+partial Alu.

The transfection steps were as follows: Hela cells were passaged and spread into 24-well plates. Cultivate using complete medium. On the next day of passage, wait for Hela cells to grow to 60% confluence, then replace the complete medium with Opti-MEM™ I medium. According to the instructions of the RNAiMAX transfection reagent, hLRE1-ORF1p+hLRE1-ORF2p-liposome complex, RNA+partial Alu+hLRE1-ORF1p liposome complex, RNA+partial Alu+hLRE1-ORF2p-lipsoome complex, RNA+hLRE1-ORF1p+hLRE1-ORF2p-liposome complex, RNA+partial Alu+hLRE1-ORF1p+hLRE1-ORF2p-liposome complex, and RNA+partial Alu-liposome complex were transfected into Hela cells separately. Set three parallels for each processing. Replace the medium with complete medium 6 hours after transfection. Continue to culture cells until they grow to about 90% confluence before passaging. After passaging, when the cells grow to about 60% confluence, repeat transfection once. Wait for the cells to grow again to about 90% confluence before proceeding with subsequent operations.

For cells transfected with hLRE1-ORF1p+hLRE1-ORF2p-liposome complex, the cells grew again to about 90% confluence and were passaged again. After passaging, when the cells grew to about 60% confluence, part of the complete culture medium was aspirated to the remaining 0.5 ml, and the control group plasmid (pBS-L1PA1-CH-mneo) was transfected. Transfection was performed using Entranster-H4000 transfection reagent. For transfection of each plate cell, 19.2 μg plasmids pBS-L1PA1-CH-mneo were added. Dilute the required plasmid with 600 μL serum-free DMEM solution and mix thoroughly; Dilute 48 μL Entranster-H4000 reagent with 600 μL serum-free DMEM solution, mix thoroughly, and let stand at room temperature for 5 minutes. Afterwards, mix the two prepared liquids thoroughly and let them stand at room temperature for 15 minutes to prepare a transfection complex. Add the transfection complex to a 24 well plate containing Hela cells transfected with hLRE1-ORF1p+hLRE1-ORF2p-liposome complex and 0.5 ml of medium per well for transfection. When the cells grew to about 90% confluence, they were passaged again. After passaging, the above operation was repeated. After the cells grew again to about 90% confluence, subsequent operations are carried out, and these cells was used as a control.

### Transfection of Direct Transfection Plasmid

Co transfect the constructed plasmid pcDNA3.1(+)eGFP+RNA+partial Alu with plasmids pBS-L1PA1-CH-mneo expressing ORF1p and ORF2p (LINE-1) into Hela cells, with 3 parallels in each group, each consisting of a 24 well plate cultured with Hela cells.

The transfection steps were as follows: Hela cells were passaged and spread into 24-well plates. On the next day of passage, transfection was performed using Entranster-H4000 transfection reagent. For transfection of each plate of cells, two plasmids were co transfected, with each plasmid transfected 19.2 μg, for a total of 38.4 μg. The required plasmids were diluted with 600 μL serum-free DMEM and mixed thoroughly. At the same time, 48 μL of Entranster-H4000 reagent was diluted with 600 μL of serum-free DMEM, and after fully mixed, it was allowed to stand for 5 min at room temperature. Then the prepared two liquids were mixed and fully mixed, and allowed to stand for 15 min at room temperature to prepare the transfection complex. The transfection complex was added to 24-well plate with Hela cells, in which contained 0.5 ml Opti-MEM medium per well, for transfection. After the cells grew to about 90% confluence, they were passaged, and the above operations were repeated after passage. After the cells grew to about 90% confluence again, samples were taken for subsequent operations.

### Experimental Grouping:

It was divided into seven groups: the group transfected with hLRE1-ORF1p+hLRE1-ORF2p-liposome complex and plasmid pBS-L1PA1-CH-mneo as the control group, the plasmid direct transfection group as the experimental group 1, the group transfected with RNA+hLRE1-ORF1p+hLRE1-ORF2p plasmid as the experimental group 2, the group transfected with RNA+partial Alu plasmid as the experimental group 3, the group transfected with RNA+partial Alu+hLRE1-ORF1p as the experimental group 4, and the group transfected with RNA+partial Alu+hLRE1-ORF2p as the experimental group 5 The group transfected with RNA+partial Alu+hLRE1-ORF1p+hLRE1-ORF2p as experimental group 6, with three parallels in each group, each consisting of a 24 well plate cultured with Hela cells.

Extraction of transfected cell DNA: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the GAPDH gene do not contain an Alu sequence and its copy number is stable, the GAPDH gene is used as a reference gene.

The upstream primer sequence for detecting the GAPDH gene is shown in Seq ID No.19 as follows: 5′-CACTGCCACCCAGAAGACTG-3′. The downstream primer sequence is shown in Seq ID No.20: 5′-CCTGCTTCACCACCTTCTTG-3′.

A primer pair 1 was designed, wherein an upstream primer sequence of the primer pair 1 is shown as Seq ID No.21: 5′-GACTTATCCATGTGCCTGTT-3′; and the downstream primer sequence is shown in Seq ID No.22: 5′-TTGGCTACGAAATGCTTG-3′. The upstream primer sequence of primer pair 1 is located in the complete Lman1 gene, further upstream of the upstream sequence of the insertion site (target site) used on the RNA prepared, not in the RNA prepared, but only in the genome, and the downstream primer sequence of primer pair 1 is located on the exogenous sequence to be inserted (the sequence to be inserted).

The primers were all obtained through chemical synthesis.

The qPCR reaction system is shown in Table 12.

TABLE 12
qPCR reaction system

| Component | Addition Amount | |
|---|---|---|
| Upstream primer (10 μM) | 1.5 | μL |
| Downstream primer (10 μM) | 1.5 | μL |
| 2 × SuperReal PreMix Plus | 25 | μL |
| Cellular DNA template | 4 | μL |
| (25 ng/μL) | | |

| | |
|---|---|
| ddH₂O without RNase | Make up to 50 μL |

The cellular DNA template was DNA extracted from the aforementioned 7 groups after transfection.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 1: pre-denaturation at 95° C. for 15 min; (denaturation at 95° C. for 10 s, annealing at 49° C. for 20 s, and extension at 72° C. for 20 s) 40 cycles. The GAPDH primers were reacted under the same conditions.

The exponential growth phases in the amplification curves of GAPDH and the detection of the insertion of the sequence to be inserted were observed and were confirmed to be approximately parallel, the obtained data were analyzed by the 2^{-ΔΔCt} method, and the results are shown in Table 13. The PCR products were verified to be correct by sequencing.

TABLE 13
Results of primer pair 1 (n = 3, x̅ ± s)

| | | | | ΔΔCt | |
|---|---|---|---|---|---|
| | | Insertion | | (mean ΔCt of | Relative |
| | | of the | | the experimental | amount |
| | | sequence | | group − mean | of copy |
| | | to be | | ΔCt of the | number |
| Ct value | GAPDH | inserted | ΔCt | control group 1) | (2^{-ΔΔCt}) |
| Control group 1 | 26.23 | N/A | 13.77 | — | — |
| plate 1 | | | | | |
| Control group 1 | 26.14 | N/A | 13.86 | — | — |
| plate 2 | | | | | |
| Control group 1 | 25.80 | N/A | 14.20 | — | — |
| plate 3 | | | | | |
| Control group 1 | | | 13.94 ± 0.19 | 0.00 ± 0.19 | 1.00 |
| plate mean | | | | | (0.88-1.14) |
| Experimental | 26.14 | 27.56 | 1.42 | — | — |
| group 1 plate 1 | | | | | |
| Experimental | 26.03 | 27.28 | 1.25 | — | — |
| group 1 plate 2 | | | | | |
| Experimental | 26.51 | 28.01 | 1.50 | — | — |
| group 1 plate 3 | | | | | |
| Experimental | | | 1.39 ± 0.10 | −12.55 ± 0.10 | 5996.90 |
| group 1 mean | | | | | (5595.30-6427.31) |
| Experimental | 26.12 | 37.39 | 11.27 | — | — |
| group 2 plate 1 | | | | | |
| Experimental | 25.94 | 38.42 | 12.48 | — | — |
| group 2 plate 2 | | | | | |
| Experimental | 26.52 | 38.02 | 11.50 | — | — |
| group 2 plate 3 | | | | | |
| Experimental | | | 11.75 ± 0.52 | −2.19 ± 0.52 | 4.56 |
| group 2 mean | | | | | (3.18-6.54) |
| Experimental | 26.87 | 34.77 | 7.90 | — | — |
| group 3 plate 1 | | | | | |
| Experimental | 26.39 | 35.15 | 8.76 | — | — |
| group 3 plate 2 | | | | | |
| Experimental | 26.16 | 35.06 | 8.90 | — | — |
| group 3 plate 3 | | | | | |
| Experimental | | | 8.52 ± 0.44 | −5.42 ± 0.44 | 42.81 |
| group 3 mean | | | | | (31.56-58.08) |
| Experimental | 26.02 | 32.63 | 6.61 | — | — |
| group 4 plate 1 | | | | | |
| Experimental | 26.15 | 32.96 | 6.81 | — | — |
| group 4 plate 2 | | | | | |
| Experimental | 26.41 | 33.80 | 7.39 | — | — |
| group 4 plate 3 | | | | | |
| Experimental | | | 6.94 ± 0.33 | −7.00 ± 0.33 | 128 |
| group 4 mean | | | | | (101.83-160.90) |
| Experimental | 25.79 | 29.09 | 3.30 | | |
| group 5 plate 1 | | | | | |
| Experimental | 25.98 | 28.43 | 2.45 | | |
| group 5 plate 2 | | | | | |
| Experimental | 26.34 | 29.42 | 3.08 | | |
| group 5 plate 3 | | | | | |
| Experimental | | | 2.94 ± 0.36 | −11.00 ± 0.36 | 2048 |
| group 5 mean | | | | | (1595.73-2628.46) |
| Experimental | 25.72 | 26.45 | 0.73 | | |
| group 6 plate 1 | | | | | |
| Experimental | 26.08 | 26.19 | 0.11 | | |
| group 6 plate 2 | | | | | |
| Experimental | 26.33 | 26.59 | 0.26 | | |
| group 6 plate 3 | | | | | |
| Experimental | | | 0.37 ± 0.26 | −13.57 ± 0.26 | 12161.22 |
| group 6 mean | | | | | (10155.69-14562.80) |

From the Table 10, it can be seen that compared with the control group (N/A calculated at 40.00), the relative copy number of experimental group 1 was significantly higher than that of the control group, with statistical significance (P<0.05). Therefore, by administering plasmids (DNA) containing upstream sequence of target site, sequence to be inserted, and downstream sequence of target site to the receiving system, the insertion of the sequence to be inserted into the genome target site can be achieved (experimental group 1). Compared with experimental group 1, the relative copy number of experimental group 6 was significantly higher than that of experimental group 1, with statistical significance (P<0.05), indicating that due to the existence of RNA splicing mechanism in eukaryotic organisms (cells), the efficiency of plasmid transcription to produce RNA with gene editing function is reduced. Therefore, the efficiency of plasmid direct transfection (experimental group 1) is lower than that of directly introducing RNA or RNP containing upstream sequence of target site, sequence to be inserted, and downstream sequence of target site (experimental group 6) into the receiving system under similar conditions. In addition, compared with the control group (N/A calculated at 40.00), the relative copy number of experimental group 2 was significantly higher than that of the control group, with statistical significance (P<0.05), indicating that RNA or RNP bound with ORF1p and/or ORF2p containing only upstream sequence of target site, sequence to be inserted, and downstream sequence of target site (experimental group 2), can also play a gene editing role, but the effect is comparably weak, indicating that RNA or RNP bound with ORF1p and/or ORF2p containing only upstream sequence of target site, sequence to be inserted, and downstream sequence of target site, can also achieve the purpose of gene editing. Compared with the control group (N/A calculated at 40.00), the relative copy number of experimental group 3 was significantly higher than that of the control group, with statistical significance (P<0.05), indicating that even without combining ORF1p and/or ORF2p, adding additional partial Alu beyond the upstream sequence of target site, sequence to be inserted, and downstream sequence of target site (experimental group 3) can still achieve the effect of gene editing, And its gene editing effect (compared to the relative copy number of the control group) is also significantly higher than that of the experimental group 2, indicating that the addition of partial Alu can improve the efficiency of gene editing. Compared with the control group (N/A calculated at 40.00), the relative copy number of experiment group 4-6 was significantly higher than that of the control group, with statistical significance (P<0.05), indicating that RNA containing upstream sequence of target site, sequence to be inserted, downstream sequence of target site, and partial Alu could bind to ORF1p, ORF2p, or ORF1p and ORF2p to produce gene editing effects. In addition, the results showed that the gene editing efficiency of experimental group 4-6 gradually improved, indicating that the binding of ORF2p has a better effect on improving gene editing efficiency than ORF1p, while the binding of ORF2p and ORF1p is better than the single binding of ORF1p or ORF2p.

Compare the gene editing efficiency of RNA production by in vitro prokaryotic transcription followed by transfection and DNA direct transfection.

TABLE 14
Results of primer pair 1 (n = 3, x̅ ± s)

| | | | | ΔΔCt (mean | |
|---|---|---|---|---|---|
| | | | | ΔCt of the | |
| | | | | experimental | |
| | | Insertion | | group 6 − mean | Relative |
| | | of sequence | | ΔCt of the | amount of |
| | | to be | | experimental | copy number |
| Ct value | GAPDH | inserted | ΔCt | group 1) | (2^{-ΔΔCt}) |
| Experimental | 26.14 | 27.56 | 1.42 | — | — |
| group 1 plate 1 | | | | | |
| Experimental | 26.03 | 27.28 | 1.25 | — | — |
| group 1 plate 2 | | | | | |
| Experimental | 26.51 | 28.01 | 1.50 | — | — |
| group 1 plate 3 | | | | | |
| Experimental | | | 1.39 ± | 0.00 ± | 1.00 |
| group 1 mean | | | 0.10 | 0.10 | (0.93-1.07) |
| Experimental | 25.72 | 26.45 | 0.73 | — | — |
| group 6 plate 1 | | | | | |
| Experimental | 26.08 | 26.19 | 0.11 | — | — |
| group 6 plate 2 | | | | | |
| Experimental | 26.33 | 26.59 | 0.26 | — | — |
| group 6 plate 3 | | | | | |
| Experimental | | | 0.37 ± | −1.02 ± | 2.03 |
| group 6 mean | | | 0.26 | 0.26 | (1.69-2.43) |

From Table 14, it can be seen that compared with Experimental group 1, the relative copy number of Experimental group 6 was significantly higher than that of Experimental group 1, with statistical significance (P<0.05). This indicates that gene editing is more efficient in some cases when specific RNA or RNP is generated in vitro through prokaryotic promoters or other methods and then introduced into the receiving system for gene editing. This may be due to the production of specific RNA or RNP in vitro, avoiding the cutting or splicing effects of the eukaryotic system on the RNA produced. It reflects the advantages of producing specific RNA or RNP in vitro and then introducing it into the receiving system such as cells, tissues, organs, or organisms, compared to directly introducing the corresponding DNA into the receiving system, in some cases.

### Embodiment 3 Inspection of the Effect of Gene Editing by Introducing RNA Produced by In Vitro Transcription into a Target System with Binding of ORF1p and/or ORF2p within the Target System

The plasmid pBS-L1PA1-CH-mneo expressing ORF1p and ORF2p contains codon optimized ORF1 and ORF2 of human L1RP, which can express hLRE1-ORF1 and hLRE1-ORF2 in cells.

Transfection of Plasmid pBS-L1PA1-CH-Mneo

The transfection steps were as follows: Hela cells were passaged and spread into 24-well plates. On the next day of passage, when the cells grow to 60% confluence, transfection was performed using Entranster-H4000 transfection reagent. For transfection of each plate of cells, 19.2 μg plasmid pBS-L1PA1-CH-mneo was transfected. The plasmid pBS-L1PA1-CH-mneo was diluted with 600 μL serum-free DMEM and mixed thoroughly. At the same time, 48 μL of Entranster-H4000 reagent was diluted with 600 μL of serum-free DMEM, and after fully mixed, it was allowed to stand for 5 min at room temperature. Then the prepared two liquids were mixed and fully mixed, and allowed to stand for 15 min at room temperature to prepare the transfection complex. The transfection complex was added to 24-well plate with Hela cells, in which contained 0.5 ml Opti-MEM medium per well, for transfection. After the cells grew to about 90% confluence, they were passaged. After passage, Hela cells were spread on a 24 well plate, and the next day of passage, further RNA transfection was carried out when the cells grew to 60% confluence. There were three parallels, each consisting of a 24 well plate for culturing Hela cells transfected with pBS-L1PA1-CH-mneo plasmid.

### RNA Transfection

Mix the RNA+partial Alu solution prepared in embodiment 2 according to the system in Table 8, then add the transfection solution system in Table 7 in equal volume ratio. Gently mix and incubate at room temperature (25° C.) for 20 minutes to obtain the RNA+partial Alu-liposome complex.

Cultivate Hela cells transfected with pBS-L1PA1-CH-mneo plasmid to 60% confluence, then replace with Opti MEM™ I medium. According to the instructions of RNAiMAX transfection reagent, add RNA+partial Alu-liposome complex to the cells for transfection, with three parallels for each group. Continue to culture cells until they grow to about 90% confluence before passaging. After passaging, repeat transfection once, and the cells grow until they fuse to about 90% fusion. Extract the cell DNA for subsequent operations. This experiment is used as the experimental group.

Using the same method, RNA+partial Alu-liposome complex was transfected into Hela cells without pBS-L1PA1-CH-mneo plasmid as the control group, with three parallels for each group.

Extraction of transfected cell DNA: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

The GAPDH gene is used as a reference gene. The upstream primer sequence for detecting the GAPDH gene is shown in Seq ID No.19. The downstream primer sequence is shown in Seq ID No.20. A primer pair 1 was designed, wherein an upstream primer sequence of the primer pair 1 is shown as Seq ID No.21; and the downstream primer sequence is shown in Seq ID No.22.

The qPCR reaction system is shown in Table 12.

The cellular DNA template was DNA extracted from the aforementioned control group or experimental groups after transfection.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 1: pre-denaturation at 95° C. for 15 min; (denaturation at 95° C. for 10 s, annealing at 49° C. for 20 s, and extension at 72° C. for 20 s) 40 cycles. The GAPDH primers were reacted under the same conditions.

The exponential growth phases in the amplification curves of GAPDH and the detection of the insertion of the sequence to be inserted were observed and were confirmed to be approximately parallel, the obtained data were analyzed by the 2^{-ΔΔCt} method, and the results are shown in Table 15. The PCR products were verified to be correct by sequencing.

TABLE 15
Results of primer pair 1 (n = 3, x̅ ± s)

| | | | | ΔΔCt (mean | |
|---|---|---|---|---|---|
| | | | | ΔCt of the | |
| | | Insertion | | experimental | Relative |
| | | of sequence | | group − mean | amount of |
| | | to be | | ΔCt of the | copy number |
| Ct value | GAPDH | inserted | ΔCt | control group) | (2^{-ΔΔCt}) |
| Control group | 26.87 | 34.77 | 7.90 | — | — |
| plate 1 | | | | | |
| Control group | 26.39 | 35.15 | 8.76 | — | — |
| plate 2 | | | | | |
| Control group | 26.16 | 35.06 | 8.90 | — | — |
| plate 3 | | | | | |
| Control group | | | 8.52 ± | 0.00 ± | 1.00 |
| plate mean | | | 0.44 | 0.44 | (0.74-1.36) |
| Experimental | 26.18 | 26.82 | 0.64 | — | — |
| group plate 1 | | | | | |
| Experimental | 25.69 | 26.53 | 0.84 | — | — |
| group plate 2 | | | | | |
| Experimental | 26.34 | 27.42 | 1.08 | — | — |
| group plate 3 | | | | | |
| Experimental | | | 0.85 ± | −7.67 ± | 203.66 |
| group mean | | | 0.18 | 0.18 | (179.77-230.72) |

As shown in Table 15, compared with the control group, the relative copy number of the experimental group was significantly higher than that of the control group, with statistical significance (P<0.05). Compared to administering specific RNA alone, the gene editing efficiency of producing ORF1p and/or ORF2p in the target system combined with specific RNA was higher. By generating ORF1p and/or ORF2p within the target system, it can assist in the gene editing of specific RNAs and improve gene editing efficiency.

Embodiment 4 Inspection of the Effect of Gene Editing by Introducing Specific RNA (3′Site is Intact Alu) Produced by In Vitro Transcription into a Target System with/without Binding of ORF1p and/or ORF2p Outside the Target System

The GALT gene encodes galactose-1-phosphate uridyltransferase, and its mutation can lead to human type I galactosamia.

Select a 360 bp sequence from the GALT gene, as shown in Seq ID No.23:

GGGGTTCGGCCCTGCCCGTAGCACAGCCAAGCCCTACCTCTCGGTTATC
TTTTCTCCCGTCACCACCCAGTAAGGTCATGTGCTTCCACCCCTGGTCG
GATGTAACGCTGCCACTCATGTCGGTCCCTGAGATCCGGGCTGTTGTTG
*ATGCATGGGCCTCAGTCACAGAGGAGCTGGGTGCCCAGTACCCTTGGG
TGCAGGTTTGTGAGGTCGCCCCTTCCCCTGGATGGGCAGGGAGGGGGTG
ATGAAGCTTTGGTTCTGGGGAGTAACATTTCTGTTTCCACAGGGTGTGG
TCAGGAGGGAGTTGACTTGGTGTCTTTTGGCTAACAGAGCTCCGTATCC
CTATCTGATAGATCTTTG.

Among them, * represents the selected insertion site (target site), before the insertion site is the upstream sequence of insertion site (upstream sequence of target site) in the GALT gene, and after the insertion site is the downstream sequence of insertion site (downstream sequence of target site) in the GALT gene. Insert an exogenous sequence at *, which is the sequence to be inserted, as shown in Seq ID No.24.

TGACTACTGAGATTACTTTGACATGTCCCACTTATTAATATCACCTTAA
GTTTGGGTTCGATTAATATTATGTAACCTGTGAACGAGATAAGATTCTA
GAGATTTAATCGAACCTTAATTCTGATTCGGTTATGTCAAAAGGTGTCT
TGA

The sequence after insertion is shown as Seq ID No.25:

GGGGTTCGGCCCTGCCCGTAGCACAGCCAAGCCCTACCTCTCGGTTATC
TTTTCTCCCGTCACCACCCAGTAAGGTCATGTGCTTCCACCCCTGGTCG
GATGTAACGCTGCCACTCATGTCGGTCCCTGAGATCCGGGCTGTTGTTG
TGACTACTGAGATTACTTTGACATGTCCCACTTATTAATATCACCTTAA
GTTTGGGTTCGATTAATATTATGTAACCTGTGAACGAGATAAGATTCTA
GAGATTTAATCGAACCTTAATTCTGATTCGGTTATGTCAAAAGGTGTCT
TGAATGCATGGGCCTCAGTCACAGAGGAGCTGGGTGCCCAGTACCCTTG
GGTGCAGGTTTGTGAGGTCGCCCCTTCCCCTGGATGGGCAGGGAGGGGG
TGATGAAGCTTTGGTTCTGGGGAGTAACATTTCTGTTTCCACAGGGTGT
GGTCAGGAGGGAGTTGACTTGGTGTCTTTTGGCTAACAGAGCTCCGTAT
CCCTATCTGATAGATCTTTG

The underline represents the sequence to be inserted as shown in Seq ID No.24.

Add the T7 promoter sequence as shown in Seq ID No.11 upstream of the sequence shown in Seq ID No.25, and the Alu sequence as shown in Seq ID No.1 downstream of the sequence shown in Seq ID No.25. The sequence after addition is shown in Seq ID No.26:

TAATACGACTCACTATAGGGGTTCGGCCCTGCCCGTAGCACAGCCAAGCCCT
ACCTCTCGGTTATCTTTTCTCCCGTCACCACCCAGTAAGGTCATGTGCTTCCACCC
CTGGTCGGATGTAACGCTGCCACTCATGTCGGTCCCTGAGATCCGGGCTGTTGTT
GTGACTACTGAGATTACTTTGACATGTCCCACTTATTAATATCACCTTAAGTTTGG
GTTCGATTAATATTATGTAACCTGTGAACGAGATAAGATTCTAGAGATTTAATCG
AACCTTAATTCTGATTCGGTTATGTCAAAAGGTGTCTTGAATGCATGGGCCTCAG
TCACAGAGGAGCTGGGTGCCCAGTACCCTTGGGTGCAGGTTTGTGAGGTCGCCCC
TTCCCCTGGATGGGCAGGGAGGGGGTGATGAAGCTTTGGTTCTGGGGAGTAACA
TTTCTGTTTCCACAGGGTGTGGTCAGGAGGGAGTTGACTTGGTGTCTTTTGGCTA

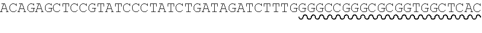

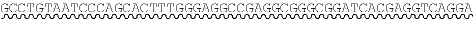

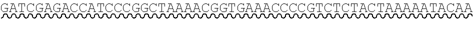

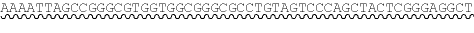

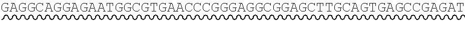

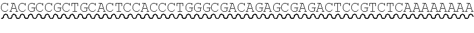

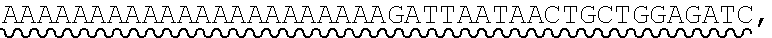

Wherein the underline represents the sequence to be inserted as shown in Seq ID No.24, the italicized bold represents the T7 promoter sequence as shown in Seq ID No.11, and the wavy line represents the Alu sequence as shown in Seq ID No.1. This sequence was obtained through chemical synthesis and named precursor DNA of RNA+Alu.

Add the T7 promoter sequence shown in Seq ID No.11 upstream of the sequence shown in Seq ID No.25, and the sequence after addition is shown in Seq ID No.27:

TAATACGACTCACTATAGGGGTTCGGCCCTGCCCGTAGCACAGCCAAGC
CCTACCTCTCGGTTATCTTTTCTCCCGTCACCACCCAGTAAGGTCATGT
GCTTCCACCCCTGGTCGGATGTAACGCTGCCACTCATGTCGGTCCCTGA
GATCCGGGCTGTTGTTGTGACTACTGAGATTACTTTGACATGTCCCACT
TATTAATATCACCTTAAGTTTGGGTTCGATTAATATTATGTAACCTGTG
AACGAGATAAGATTCTAGAGATTTAATCGAACCTTAATTCTGATTCGGT
TATGTCAAAAGGTGTCTTGAATGCATGGGCCTCAGTCACAGAGGAGCTG
GGTGCCCAGTACCCTTGGGTGCAGGTTTGTGAGGTCGCCCCTTCCCCTG
GATGGGCAGGGAGGGGGTGATGAAGCTTTGGTTCTGGGGAGTAACATTT
CTGTTTCCACAGGGTGTGGTCAGGAGGGAGTTGACTTGGTGTCTTTTGG
CTAACAGAGCTCCGTATCCCTATCTGATAGATCTTTG,

Wherein the underline represents the sequence to be inserted as shown in Seq ID No.24, and the italicized bold represents the T7 promoter sequence as shown in Seq ID No.11. This sequence was obtained through chemical synthesis and named as the precursor DNA of RNA.

According to the instructions of the MEGAscript™ T7 Transcription Kit, the linear precursor DNA of RNA+Alu or precursor DNA of RNA were transcribed to obtain corresponding RNA. Then, the residual DNA was degraded by DNase from the kit and resuspended in RNase free water. The RNA concentration was measured by UV spectrophotometer, and the concentration of RNA+Alu solution or RNA solution was further configured to 100 ng/μL by adding RNase free water.

The RNA+Alu obtained from the above transcription belongs to the RNA framework structure in FIG. 4, where the ORF2p functional initiating (initiation) part is intact short interspersed element (SINE). The RNA transcribed above belongs to the RNA framework structure shown in FIG. 3.

Resuspend hLRE1-ORF1p and hLRE1-ORF2p prepared in embodiment 1 with Opti MEM solution pre added with 1 U/μL RNA enzyme inhibitor, respectively, into 500 ng/μL of hLRE1-ORF1p solution or hLRE1-ORF2p solution.

Prepare RNP of RNA or RNA+Alu bound with ORF1p and/or ORF2p: RNA+hLRE1-ORF1p+hLRE1-ORF2p, RNA+Alu+hLRE1-ORF1p+hLRE1-ORF2p. Follow the steps below.

Configure the reaction system as shown in Table 16:

TABLE 16
Reaction System

| Component | Addition Amount | |
|---|---|---|
| hLRE1-ORF1p solution | 3.0 | μL |
| RNA solution or RNA + Alu solution | 21.0 | μL |

| | |
|---|---|
| Opti-MEM ™ I medium | Make up to 50 μL |

Mix the components gently and incubate the reaction system at room temperature (25° C.) for 10 minutes to obtain RNA+hLRE1-ORF1p solution and RNA+Alu+hLRE1-ORF1p solution, respectively.

Then, mix the RNA+hLRE1-ORF1p solution and RNA+Alu+hLRE1-ORF1p solution according to the system shown in Table 17.

TABLE 17
Reaction System

| Component | Addition Amount | |
|---|---|---|
| RNA + hLRE1-ORF1p solution or | 50.0 | μL |
| RNA + Alu + hLRE1-ORF1p solution | | |
| hLRE1-ORF2p solution | 5.5 | μL |

| | |
|---|---|
| Opti-MEM ™ I medium | Make up to 75 μL |

After gently mixing each component, the reaction system was incubated at room temperature (25° C.) for 10 minutes to obtain RNA+hLRE1-ORF1p+hLRE1-ORF2p solution and RNA+Alu+hLRE1-ORF1p+hLRE1-ORF2p solution, respectively.

Mix RNA+hLRE1-ORF1p+hLRE1-ORF2p solution or RNA+Alu+hLRE1-ORF1p+hLRE1-ORF2p solution in equal volume ratio with the transfection solution system prepared in Table 7. Gently mix and incubate at room temperature (25° C.) for 20 minutes to obtain RNA+hLRE1-ORF1p+hLRE1-ORF2p-liposome complex or RNA+Alu+hLRE1-ORF1p+hLRE1-ORF2p-liposome complex.

Prepare RNA+Alu-liposome complex transfection solutions without hLRE1-ORF1p and hLRE1-ORF2p, and the reaction system is shown in Table 18:

TABLE 18
Reaction System

| Component | Addition Amount |
|---|---|
| RNA + Alu solution | 21.0 μL |
| Opti-MEM ™ I medium | Make up to 75 μL |

Gently mix the various components and incubate the reaction system at room temperature (25° C.) for 10 minutes. Then, add the transfection solutions as shown in Table 7 in equal volume ratios. Gently mix and incubate at room temperature (25° C.) for 20 minutes to obtain the RNA+Alu-liposome complex.

Construction of direct transfection plasmid pcDNA3.1(+)eGFP+RNA+Alu

Connect the sequence shown in Seq ID No.1 after the sequence shown in Seq ID No.25, to obtain the sequence shown in Seq ID No.28:

GGGGTTCGGCCCTGCCCGTAGCACAGCCAAGCCCTACCTCTCGGTTATCTTT
TCTCCCGTCACCACCCAGTAAGGTCATGTGCTTCCACCCCTGGTCGGATGTAACG
CTGCCACTCATGTCGGTCCCTGAGATCCGGGCTGTTGTTGTGACTACTGAGATTA
CTTTGACATGTCCCACTTATTAATATCACCTTAAGTTTGGGTTCGATTAATATTAT
GTAACCTGTGAACGAGATAAGATTCTAGAGATTTAATCGAACCTTAATTCTGATT
CGGTTATGTCAAAAGGTGTCTTGAATGCATGGGCCTCAGTCACAGAGGAGCTGG
GTGCCCAGTACCCTTGGGTGCAGGTTTGTGAGGTCGCCCCTTCCCCTGGATGGGC
AGGGAGGGGGTGATGAAGCTTTGGTTCTGGGGAGTAACATTTCTGTTTCCACAGG
GTGTGGTCAGGAGGGAGTTGACTTGGTGTCTTTTGGCTAACAGAGCTCCGTATCC

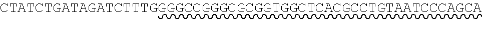

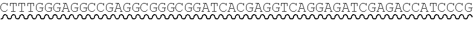

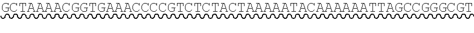

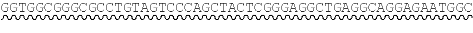

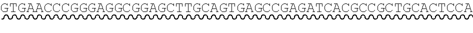

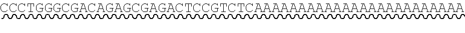

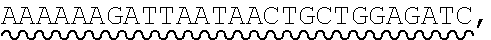

Wherein the underlined represents the sequence to be inserted as shown in Seq ID No.24; The wavy line represents a partial Alu sequence as shown in Seq ID No.1.

The sequence shown in Seq ID No.28 was chemically synthesized and constructed into the pcDNA3.1(+)eGFP vector through homologous recombination linkage, so that the sequence is directly connected downstream of the CMV promoter in the vector, and there are no other sequences between the sequence and the CMV promoter. The plasmid obtained was named pcDNA3.1(+)eGFP+RNA+Alu.

The specific steps are:

1. Design primers to amplify the sequence shown in Seq ID No.28, where the forward primer sequence is as shown in Seq ID No.29: 5′-CTATATAAGCAGAGCTGGGGTTCGGCCCT-3′, and the reverse primer sequence is as shown in Seq ID No.16: 5′-CTCTAGTTAGCCAGAGGATCTCCAGCAGTTAT-3′. Perform PCR amplification on the sequence shown in Seq ID No.28, and the reaction system is shown in Table 19:

TABLE 19
Reaction System

| Component | Addition Amount | |
|---|---|---|
| 2 × KOD PCR Master mix | 25 | μL |
| forward primer (10 μM) | 1 | μL |
| reverse primer (10 μM) | 1 | μL |
| synthesized sequence (10 pg-200 ng) | ~1 | μL |
| ddH₂O | ~22 | μL |

The amplification conditions are: 94° C. for 2 minutes; Perform 40 cycles at 98° C. for 10 seconds, 60° C. for 10 seconds, and 68° C. for 2 seconds; 68° C. for 5 minutes.

The amplification product was obtained by gel electrophoresis, extraction and purification with conventional methods. The two sides of the synthesized sequence in the amplification product were added with the sequence homologous to pcDNA3.1(+)eGFP vector.

2. Design PCR primers to amplify the pcDNA3.1(+)eGFP vector, with a forward primer as shown in Seq ID No.17: 5′-AATAACTGCTGGAGATCCTCTGGCTAACTAGAG-3′ and a reverse primer sequence as shown in Seq ID No.30: 5′-AGGGCCGAACCCCAGCTCTGCTTATATAG-3′. Perform PCR amplification on the pcDNA3.1(+)eGFP vector, and the reaction system is shown in Table 20:

TABLE 20
Reaction System

| Component | Addition Amount | |
|---|---|---|
| 2 × KOD PCR Master mix | 25 | μL |
| forward primer (10 μM) | 1 | μL |
| reverse primer (10 μM) | 1 | μL |
| pcDNA3.1(+)eGFP plasmid template (10 pg-200 ng) | ~1 | μL |
| ddH₂O | ~22 | μL |

The amplification conditions are: 94° C. for 2 minutes; Perform 40 cycles at 98° C. for 10 seconds, 60° C. for 10 seconds, and 68° C. for 6 seconds; 68° C. for 5 minutes.

The pcDNA3.1(+)eGFP plasmid vector was obtained by gel electrophoresis, extraction and purification with conventional methods. The two sides of the plasmid vector had homologous sequences with the synthesized sequence.

3. Use a one-step rapid cloning kit to connect the amplified product to the amplified pcDNA3.1(+)eGFP vector. Follow the instructions in the kit for specific steps, and the reaction system is shown in Table 11.

4. Transfect the recombinant product into competent cells (DH5a), pick the colonies on plate and sequence. After sequencing, extract the plasmid to obtain pcDNA3.1(+)eGFP-RNA+Alu.

### Transfection

### The Experimental Group of RNA Vector Binding/not Binding ORF1p and ORF2p In Vitro:

Human glioma U251 cells were passaged and spread into 24-well plates. Cultivate using complete medium. On the next day of passage, wait for human glioma U251 cells to grow to 60% confluence, then replace the complete medium with Opti-MEM™ I medium. According to the instructions of the RNAiMAX transfection reagent, RNA+hLRE1-ORF1p+hLRE1-ORF2p-liposome complex, RNA+Alu+hLRE1-ORF1p+hLRE1-ORF2p-liposome complex, RNA+Alu-liposome complex, and RNA+partial Alu-liposome complex were transfected into human glioma U251 cells separately. Set three parallels for each processing. Replace the medium with complete medium 6 hours after transfection. Continue to culture cells until they grow to about 90% confluence before passaging. After passaging, when the cells grow to about 60% confluence, repeat transfection once. Wait for the cells to grow again to about 90% confluence before proceeding with subsequent operations.

### The Control Group:

Using the above method, the hLRE1-ORF1p+hLRE1-ORF2p-liposome complex prepared in embodiment 2 was transfected into U251 cells, when the cells grew again to about 90% confluence, they were passaged again. After passaging to about 60% confluence, some of the complete medium was aspirated to the remaining 0.5 ml, and the control group plasmid (pBS-L1PA1-CH-mneo) was transfected. Transfection was performed using Entranster-H4000 transfection reagent. For transfection of each plate of cells, 19.2 μg plasmid pBS-L1PA1-CH-mneo was transfected. The required plasmids were diluted with 600 μL serum-free DMEM and mixed thoroughly. At the same time, 48 μL of Entranster-H4000 reagent was diluted with 600 μL of serum-free DMEM, and after fully mixed, it was allowed to stand for 5 min at room temperature. Then the prepared two liquids were mixed and fully mixed, and allowed to stand for 15 min at room temperature to prepare the transfection complex. Add the transfection complex to a 24 well plate containing human glioma cell U251 that has been transfected with hLRE1-ORF1p+hLRE1-ORF2p-liposome complex and contains 0.5 ml of medium per well for transfection. When the cells grew to about 90% confluence, they were passaged again. After passaging, the above operation was repeated. After the cells grew again to about 90% confluence, subsequent operations were carried out.

### Transfection of Direct Transfection Plasmid

Co transfect the constructed plasmid pcDNA3.1(+)eGFP+RNA+Alu with plasmids pBS-L1PA1-CH-mneo expressing ORF1p and ORF2p into human glioma U251 cells, with 3 parallels in each group, each consisting of a 24 well plate cultured with human glioma U251 cells.

The transfection steps were as follows: human glioma U251 cells were passaged and spread into 24-well plates. On the next day of passage, when the cells grew to about 60% confluence, transfection was performed using Entranster-H4000 transfection reagent. For transfection of each plate of cells, two plasmids were co transfected, with each plasmid transfected 19.2 μg, for a total of 38.4 μg. The required plasmids were diluted with 600 μL serum-free DMEM and mixed thoroughly. At the same time, 48 μL of Entranster-H4000 reagent was diluted with 600 μL of serum-free DMEM, and after fully mixed, it was allowed to stand for 5 min at room temperature. Then the prepared two liquids were mixed and fully mixed, and allowed to stand for 15 min at room temperature to prepare the transfection complex. The transfection complex was added to 24-well plate with human glioma U251 cells, in which contained 0.5 ml Opti-MEM medium per well, for transfection. After the cells grew to about 90% confluence, they were passaged, and the above operations were repeated after passage. After the cells grew to about 90% confluence again, samples were taken for subsequent operations.

### Experimental Grouping:

It was divided into five groups: the group transfected with hLRE1-ORF1p+hLRE1-ORF2p and plasmid pBS-L1PA1-CH-mneo was used as the control group, the plasmid direct transfection group was used as the experimental group 1, the group transfected with RNA+hLRE1-ORF1p+hLRE1-ORF2p was used as the experimental group 2, the group transfected with RNA+Alu was used as the experimental group 3, and the group transfected with RNA+Alu+hLRE1-ORF1p+hLRE1-ORF2p was used as the experimental group 4. There were three parallels in each group, each of which was a 24 well plate cultured with human glioma cell U251.

Extraction of transfected cell DNA in each group: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the copy number of GAPDH gene is stable, the GAPDH gene was used as a reference gene.

The upstream primer sequence for detecting the GAPDH gene is shown in Seq ID No.19. The downstream primer sequence is shown in Seq ID No.20.

A primer pair 2 was designed, wherein an upstream primer sequence of the primer pair 2 is shown as Seq ID No.31: 5′-CCCCAGTACGATAGCACC-3′; and the downstream primer sequence is shown in Seq ID No.32: 5′-GACATAACCGAATCAGAATT-3′. The upstream primer sequence of primer pair 2 is located in the complete GALT gene, further upstream of the upstream sequence of the insertion site (target site) used on the RNA prepared, not in the RNA prepared, but only in the genome, and the downstream primer sequence of primer pair 2 is located on the exogenous sequence to be inserted (the sequence to be inserted).

The primers were all obtained through chemical synthesis.

The qPCR reaction system is shown in Table 21.

TABLE 21
qPCR reaction system

| Component | Addition Amount | |
|---|---|---|
| Upstream primer (10 μM) | 1.5 | μL |
| Downstream primer (10 μM) | 1.5 | μL |
| 2 × SuperReal PreMix Plus | 25 | μL |
| Cellular DNA template (25 ng/μL) | 4 | μL |

| | |
|---|---|
| ddH₂O without RNase | Make up to 50 μL |

The cellular DNA template was DNA extracted from the aforementioned 5 groups after transfection.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 2: pre-denaturation at 95° C. for 15 min; (denaturation at 95° C. for 10 s, annealing at 46° C. for 20 s, and extension at 72° C. for 20 s) 40 cycles. The GAPDH primers were reacted under the same conditions.

The exponential growth phases in the amplification curves of GAPDH and the detection of the insertion of the sequence to be inserted were observed and were confirmed to be approximately parallel, the obtained data were analyzed by the 2^{-ΔΔt} method, and the results are shown in Table 22. The PCR products were verified to be correct by sequencing.

TABLE 22
Results of primer pair 2 (n = 3, x̅ ± s)

| | | | | ΔΔCt (mean | |
|---|---|---|---|---|---|
| | | | | ΔCt of the | |
| | | Insertion | | experimental | Relative |
| | | of sequence | | group − mean | amount of |
| | | to be | | ΔCt of the | copy number |
| Ct value | GAPDH | inserted | ΔCt | control group) | (2^{-ΔΔCt}) |
| Control group | 26.27 | N/A | 13.73 | — | — |
| plate 1 | | | | | |
| Control group | 26.00 | N/A | 14.00 | — | — |
| plate 2 | | | | | |
| Control group | 26.44 | N/A | 13.56 | — | — |
| plate 3 | | | | | |
| Control group | | | 13.76 ± | 0.00 ± | 1.00 |
| mean | | | 0.18 | 0.18 | (0.88-1.13) |
| Experimental | 26.38 | 28.17 | 1.79 | — | — |
| group 1 plate 1 | | | | | |
| Experimental | 25.77 | 27.32 | 1.55 | — | — |
| group 1 plate 2 | | | | | |
| Experimental | 26.51 | 27.73 | 1.22 | — | — |
| group 1 plate 3 | | | | | |
| Experimental | | | 1.52 ± | −12.24 ± | 4837.35 |
| group 1 mean | | | 0.23 | 0.23 | (4124.49-5673.41) |
| Experimental | 25.68 | 37.80 | 12.12 | — | — |
| group 2 plate 1 | | | | | |
| Experimental | 26.45 | 38.27 | 11.82 | — | — |
| group 2 plate 2 | | | | | |
| Experimental | 26.34 | 38.78 | 12.44 | — | — |
| group 2 plate 3 | | | | | |
| Experimental | | | 12.13 ± | −1.63 ± | 3.10 |
| group 2 mean | | | 0.25 | 0.25 | (2.60-3.68) |
| Experimental | 26.50 | 35.42 | 8.92 | — | — |
| group 3 plate 1 | | | | | |
| Experimental | 26.17 | 35.62 | 9.45 | — | — |
| group 3 plate 2 | | | | | |
| Experimental | 26.62 | 36.03 | 9.41 | — | — |
| group 3 plate 3 | | | | | |
| Experimental | | | 9.26 ± | −4.50 ± | 22.63 |
| group 3 mean | | | 0.24 | 0.24 | (19.16-26.72) |
| Experimental | 26.14 | 26.64 | 0.50 | — | — |
| group 4 plate 1 | | | | | |
| Experimental | 26.53 | 27.00 | 0.47 | — | — |
| group 4 plate 2 | | | | | |
| Experimental | 25.77 | 26.47 | 0.70 | — | — |
| group 4 plate 3 | | | | | |
| Experimental | | | 0.56 ± | −13.20 ± | 9410.14 |
| group 4 mean | | | 0.10 | 0.10 | (8779.97-10085.54) |

From the Table 22, it can be seen that compared with the control group (N/A calculated at 40.00), the relative copy number of experimental group 1 was significantly higher than that of the control group, with statistical significance (P<0.05). Therefore, by administering plasmids (DNA) containing upstream sequence of target site, sequence to be inserted, and downstream sequence of target site to the receiving system, the insertion of the sequence to be inserted into the genome target site can be achieved (experimental group 1). Compared with experimental group 1, the relative copy number of experimental group 4 was significantly higher than that of experimental group 1, with statistical significance (P<0.05), indicating that due to the existence of RNA splicing mechanism in eukaryotic organisms (cells), the efficiency of plasmid transcription to produce RNA with gene editing function is reduced. Therefore, the efficiency of plasmid direct transfection (experimental group 1) is lower than that of directly introducing RNA or RNP containing upstream sequence of target site, sequence to be inserted, and downstream sequence of target site (experimental group 4) into the receiving system under similar conditions. In addition, compared with the control group (N/A calculated at 40.00), the relative copy number of experimental group 2 was significantly higher than that of the control group, with statistical significance (P<0.05), indicating that RNA or RNP bound with ORF1p and/or ORF2p containing only upstream sequence of target site, sequence to be inserted, and downstream sequence of target site (experimental group 2), can also play a gene editing role, but the effect is comparably weak, indicating that RNA or RNP bound with ORF1p and/or ORF2p containing only upstream sequence of target site, sequence to be inserted, and downstream sequence of target site, can also achieve the purpose of gene editing. Compared with the control group (N/A calculated at 40.00), the relative copy number of experimental group 3 was significantly higher than that of the control group, with statistical significance (P<0.05), indicating that even without combining ORF1p and/or ORF2p, adding additional partial Alu beyond the upstream sequence of target site, sequence to be inserted, and downstream sequence of target site (experimental group 3) can still achieve the effect of gene editing, And its gene editing effect (compared to the relative copy number of the control group) is also significantly higher than that of the experimental group 2, indicating that the addition of entire Alu can improve the efficiency of gene editing. Compared with the control group (N/A calculated at 40.00), the relative copy number of experiment group 4 was significantly higher than that of the control group, with statistical significance (P<0.05), indicating that RNA containing upstream sequence of target site, sequence to be inserted, downstream sequence of target site, and entire Alu could bind to ORF1p, ORF2p, or ORF1p and ORF2p to produce gene editing effects. The above results also indicate that the entire Alu sequence can effectively improve the gene editing effect of the present invention.

Compare the gene editing efficiency between in vitro prokaryotic transcription of RNA and binding to ORF1p and ORF2p, followed by transfection, and direct DNA transfection.

TABLE 23
Results of primer pair 2 (n = 3, x̅ ± s)

| | | | | ΔΔCt (mean | |
|---|---|---|---|---|---|
| | | | | ΔCt of the | |
| | | | | experimental | |
| | | Insertion | | group 4 − mean | Relative |
| | | of sequence | | ΔCt of the | amount of |
| | | to be | | experimental | copy number |
| Ct value | GAPDH | inserted | ΔCt | group 1) | (2^{-ΔΔCt}) |
| Experimental | 26.38 | 28.17 | 1.79 | — | — |
| group 1 plate 1 | | | | | |
| Experimental | 25.77 | 27.32 | 1.55 | — | — |
| group 1 plate 2 | | | | | |
| Experimental | 26.51 | 27.73 | 1.22 | — | — |
| group 1 plate 3 | | | | | |
| Experimental | | | 1.52 ± | 0.00 ± | 1.00 |
| group 1 mean | | | 0.23 | 0.23 | (0.85-1.17) |
| Experimental | 26.14 | 26.64 | 0.50 | — | — |
| group 4 plate 1 | | | | | |
| Experimental | 26.53 | 27.00 | 0.47 | — | — |
| group 4 plate 2 | | | | | |
| Experimental | 25.77 | 26.47 | 0.70 | — | — |
| group 4 plate 3 | | | | | |
| Experimental | | | 0.56 ± | −0.96 ± | 1.95 |
| group 4 mean | | | 0.10 | 0.10 | (1.82-2.08) |

From Table 23, it can be seen that compared with Experimental group 1, the relative copy number of Experimental group 4 was significantly higher than that of Experimental group 1, with statistical significance (P<0.05). This indicates that gene editing is more efficient in some cases when specific RNA or RNP is generated in vitro through prokaryotic promoters or other methods and then introduced into the receiving system for gene editing. This may be due to the production of specific RNA or RNP in vitro, avoiding the cutting or splicing effects of the eukaryotic system on the RNA produced. It reflects the advantages of producing specific RNA or RNP in vitro and then introducing it into the receiving system such as cells, tissues, organs, or organisms, compared to directly introducing the corresponding DNA into the receiving system, in some cases.

Embodiment 5 Inspection of the Effect of Gene Editing by Introducing RNA (3′ Part is the RNA Sequence Corresponding to the 3′UTR of Long Interspersed Element (Partial Long Interspersed Element RNA)) Produced by In Vitro Transcription into a Target System with/without Binding of ORF1p and/or ORF2p Outside the Target System

Select a 400 bp sequence from the Lman1 gene in the human genome, as shown in Seq ID No.33:

GAGATTCACTGCCTTAGTCTCATGTAGTCTCGTGTAGTCTTTTGAGTAA
ATAACATAAAGTATCTCAAGACTTTTTCATAACTTGATATTATTTTAGT
CTTCCTGAATTTTTAAATATTGAAAAGCTGAGTGTCTTGTCTGTTTTCC
TCCCCCTTACACTATAGTGACGGGGCTAGTCAAGCTTTGGCAAGTTGCC
AGAGGGACTTCCGCAACAAACCCTATCCTGTCCGAGCAA*AGATTACCT
ATTACCAGAACACACTGACAGTAAGTAACATCTATTTAGAGAGAATCAA
ATAAACAATGTTACAGTATCACTTTTCATTTTGAATTTTTGATAGAAAT
TAAATGCACTTAAATTTGGATATGCTTACATACTCTTCATTGTTACTCT
AAGAGAACG,

Wherein * represents the selected insertion site (target site), before the insertion site is the upstream sequence of insertion site (upstream sequence of target site) in the Lman1 gene, and after the insertion site is the downstream sequence of insertion site (downstream sequence of target site) in the Lman1 gene. The sequence Seq ID No.33 is 5 bp less than Seq ID No.8 at the 5′ end, in order to increase the transcription efficiency of the Sp6 promoter.

Insert an exogenous sequence at *, which is the sequence to be inserted, as shown in Seq ID No.9.

The sequence after insertion is shown as Seq ID No.34:

GAGATTCACTGCCTTAGTCTCATGTAGTCTCGTGTAGTCTTTTGAGTAA
ATAACATAAAGTATCTCAAGACTTTTTCATAACTTGATATTATTTTAGT
CTTCCTGAATTTTTAAATATTGAAAAGCTGAGTGTCTTGTCTGTTTTCC
TCCCCCTTACACTATAGTGACGGGGCTAGTCAAGCTTTGGCAAGTTGCC
AGAGGGACTTCCGCAACAAACCCTATCCTGTCCGAGCAAAGGTGCCTGC
ACATACTGCATGTGAGAGTCTGGAGACGCCAGACTGTTCTGAGTCCTGA
CCTGCTCAGGGGTGAGGTCCCTCTGAGCCTGAGCAAGCATTTCGTAGCC
AACCATGAATTTCCGGACAGTGGCAGAGCGCAGGAGCGGAGGAGATTAC
CTATTACCAGAACACACTGACAGTAAGTAACATCTATTTAGAGAGAATC
AAATAAACAATGTTACAGTATCACTTTTCATTTTGAATTTTTGATAGAA
ATTAAATGCACTTAAATTTGGATATGCTTACATACTCTTCATTGTTACT
CTAAGAGAACG,

Wherein the underline represents the sequence to be inserted as shown in Seq ID No.9.

Add the Sp6 promoter sequence (Seq ID No.35: ATTTAGGTGACACTATA) upstream of the sequence shown in Seq ID No.34, and the LINE-3′UTR sequence shown in Seq ID No.36 downstream of the sequence shown in Seq ID No.34:

ACAATGAGATCACATGGACACAGGAAGGGGAATATCACACTCTGGGGAC
TGTGGTGGGGTCGGGGGAGGGGGGAGGGGTAGCATTGGGAGATATACCT
AATGCTAGATGACACATTAGTGGGTGCAGCGCACCAGCATGGCACATGT
ATACATATGTAACTAACCTGCACAATGTGCACATGTACCCTAAAACTTA
GAGTATAATTAAAAAAAAAAAAAAAAAAAAAAAAAAAAAAAAAAAAA,

After addition, as shown in Seq ID No.37:

ATTTAGGTGACACTATAGAGATTCACTGCCTTAGTCTCATGTAGTCTCGTGTA
GTCTTTTGAGTAAATAACATAAAGTATCTCAAGACTTTTTCATAACTTGATATTAT
TTTAGTCTTCCTGAATTTTTAAATATTGAAAAGCTGAGTGTCTTGTCTGTTTTCCT
CCCCCTTACACTATAGTGACGGGGCTAGTCAAGCTTTGGCAAGTTGCCAGAGGG
ACTTCCGCAACAAACCCTATCCTGTCCGAGCAAAGGTGCCTGCACATACTGCATG
TGAGAGTCTGGAGACGCCAGACTGTTCTGAGTCCTGACCTGCTCAGGGGTGAGG
TCCCTCTGAGCCTGAGCAAGCATTTCGTAGCCAACCATGAATTTCCGGACAGTGG
CAGAGCGCAGGAGCGGAGGAGATTACCTATTACCAGAACACACTGACAGTAAGT
AACATCTATTTAGAGAGAATCAAATAAACAATGTTACAGTATCACTTTTCATTTT
GAATTTTTGATAGAAATTAAATGCACTTAAATTTGGATATGCTTACATACTCTTC

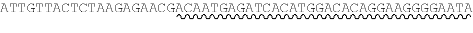

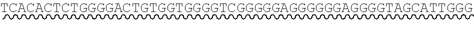

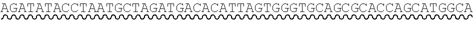

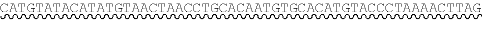

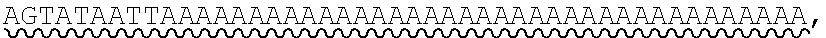

Wherein the underline represents the sequence to be inserted as shown in Seq ID No.9, the italicized bold represents the Sp6 promoter sequence as shown in Seq ID No.35, and the wavy line represents the LINE-3′UTR sequence as shown in Seq ID No.36. The sequence was obtained through chemical synthesis and named precursor DNA of RNA+LINE-3′UTR (RNA).

According to the instructions of the MEGAscript™ SP6 Transcription Kit, the linear precursor DNA of RNA+LINE-3′UTR (RNA) were transcribed to obtain corresponding RNA. Then, the residual DNA was degraded by DNase from the kit and resuspended in RNase free water. The RNA concentration was measured by UV spectrophotometer, and the concentration of RNA+LINE-3′UTR (RNA) solution was further configured to 100 ng/μL by adding RNase free water.

The RNA+LINE-3′UTR (RNA) obtained from the above transcription belongs to the RNA framework structure in FIG. 4, where the ORF2p functional initiating part is the partial long interspersed element RNA.

Resuspend hLRE1-ORF1p and hLRE1-ORF2p prepared in embodiment 1 with Opti MEM solution pre added with 1 U/μL RNA enzyme inhibitor, respectively, into 500 ng/μL of hLRE1-ORF1p solution or hLRE1-ORF2p solution.

Prepare RNP of RNA+LINE-3′UTR (RNA) bound with ORF1p and/or ORF2p: RNA+LINE-3′UTR (RNA)+hLRE1-ORF1p+hLRE1-ORF2p. Follow the steps below.

Configure the reaction system as shown in Table 24:

TABLE 24
Reaction System

| Component | Addition Amount | |
|---|---|---|
| RNA + LINE-3′UTR (RNA) solution | 21.0 | μL |
| hLRE1-ORF2p solution | 5.5 | μL |

| | |
|---|---|
| Opti-MEM ™ I medium | Make up to 50 μL |

Mix the components gently and incubate the reaction system at room temperature (25° C.) for 10 minutes to obtain RNA+LINE-3′UTR (RNA)+hLRE1-ORF2p solution.

Then, mix the RNA+LINE-3′UTR (RNA)+hLRE1-ORF2p solution according to the system shown in Table 25.

TABLE 25
Reaction System

| Component | Addition Amount | |
|---|---|---|
| RNA + LINE-3′UTR (RNA) + | 50.0 | μL |
| hLRE1-ORF2p solution | | |
| hLRE1-ORF1p solution | 3.0 | μL |

| | |
|---|---|
| Opti-MEM ™ I medium | Make up to 75 μL |

After gently mixing each component, the reaction system was incubated at room temperature (25° C.) for 10 minutes to obtain RNA+LINE-3′UTR (RNA)+hLRE1-ORF1p+hLRE1-ORF2p solution.

Mix RNA+LINE-3′UTR (RNA)+hLRE1-ORF1p+hLRE1-ORF2p solution in equal volume ratio with the transfection solution system prepared in Table 7. Gently mix and incubate at room temperature (25° C.) for 20 minutes to obtain RNA+LINE-3′UTR (RNA)+hLRE1-ORF1p+hLRE1-ORF2p-liposome complex.

### Transfection

Firstly, Hela cells were passaged and placed on a 24 well plate, cultured in complete medium. The next day after passaging, when cells grew to about 60% confluence, replacing the medium with Opti MEM™ I medium. According to the instructions of the RNAiMAX transfection reagent, RNA+LINE-3′UTR (RNA)+hLRE1-ORF1p+hLRE1-ORF2p-liposome complex was added to Hela cells for transfection, with three parallels for each group. Continue to culture cells until they grew to about 90% confluence before passaging. After passaging, repeat transfection once (following the previous steps). After the cells grew again to about 90% confluence, proceeded with subsequent operations.

### Experimental Grouping

Take the control group in embodiment 2 as the control group, and RNA+LINE-3′UTR (RNA)+hLRE1-ORF1p+hLRE1-ORF2p as the experimental group.

Extraction of transfected cell DNA in each group: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the GAPDH gene do not contain an Alu sequence and its copy number is stable, the GAPDH gene was used as a reference gene.

The upstream primer sequence for detecting the GAPDH gene is shown in Seq ID No.19 as follows: 5′-CACTGCCACCCAGAAGACTG-3′. The downstream primer sequence is shown in Seq ID No.20: 5′-CCTGCTTCACCACCTTCTTG-3′.

A primer pair 1 was designed, wherein an upstream primer sequence of the primer pair 1 is shown as Seq ID No.21: 5′-GACTTATCCATGTGCCTGTT-3′; and the downstream primer sequence is shown in Seq ID No.22: 5′-TTGGCTACGAAATGCTTG-3′. The upstream primer sequence of primer pair 1 is located in the complete Lman1 gene, further upstream of the upstream sequence of the insertion site (target site) used on the RNA prepared, not in the RNA prepared, but only in the genome, and the downstream primer sequence of primer pair 1 is located on the exogenous sequence to be inserted (the sequence to be inserted).

The primers were all obtained through chemical synthesis.

The qPCR reaction system is shown in Table 26.

TABLE 26
qPCR reaction system

| Component | Addition Amount | |
|---|---|---|
| Upstream primer (10 μM) | 1.5 | μL |
| Downstream primer (10 μM) | 1.5 | μL |
| 2 × SuperReal PreMix Plus | 25 | μL |
| Cellular DNA template (25 ng/μL) | 4 | μL |

| | |
|---|---|
| ddH₂O without RNase | Make up to 50 μL |

The cell DNA template was extracted from the transfected cells in the experimental group mentioned above.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 1: pre-denaturation at 95° C. for 15 min; (denaturation at 95° C. for 10 s, annealing at 49° C. for 20 s, and extension at 72° C. for 20 s) 40 cycles. The GAPDH primers were reacted under the same conditions.

The exponential growth phases in the amplification curves of GAPDH and the detection of the insertion of the sequence to be inserted were observed and were confirmed to be approximately parallel, the obtained data were analyzed by the 2^{-ΔΔCt} method, and the results are shown in Table 27. The PCR products were verified to be correct by sequencing.

TABLE 27
Results of primer pair 1 (n = 3, x̅ ± s)

| | | | | ΔΔCt (mean | |
|---|---|---|---|---|---|
| | | | | ΔCt of the | |
| | | Insertion | | experimental | Relative |
| | | of sequence | | group − mean | amount of |
| | | to be | | ΔCt of the | copy number |
| Ct value | GAPDH | inserted | ΔCt | control group) | (2^{-ΔΔCt}) |
| Control group | 26.23 | N/A | 13.77 | — | — |
| plate 1 | | | | | |
| Control group | 26.14 | N/A | 13.86 | — | — |
| plate 2 | | | | | |
| Control group | 25.80 | N/A | 14.20 | — | — |
| plate 3 | | | | | |
| Control group | | | 13.94 ± | 0.00 ± | 1.00 |
| mean | | | 0.19 | 0.19 | (0.88-1.14) |
| Experimental | 26.01 | 36.62 | 10.61 | — | — |
| group plate 1 | | | | | |
| Experimental | 26.37 | 37.55 | 11.18 | — | — |
| group plate 2 | | | | | |
| Experimental | 26.25 | 37.03 | 10.78 | — | — |
| group plate 3 | | | | | |
| Experimental | | | 10.86 ± | −3.08 ± | 8.46 |
| group mean | | | 0.24 | 0.24 | (7.16-9.99) |

From Table 27, it can be seen that compared with the control group, the relative copy number of the experimental group was significantly higher than that of the control group, with statistical significance (P<0.05). This indicates that gene editing can be effective by connecting the corresponding RNA sequence of 3′UTR of long interspersed element (LINE-1) to the RNA framework and producing corresponding specific RNA or RNP in vitro before introducing it into the receiving system. In addition, due to the fact that the corresponding RNA sequences of the 3′UTR of long interspersed element can achieve gene editing effects, the complete long interspersed element RNA (mainly the ORF1p and ORF2p coding sequences plus its corresponding RNA sequence of the 3′UTR) can also achieve theoretically. Meanwhile, this embodiment also demonstrates that the Sp6 promoter can participate in the production of RNA in vitro.

Embodiment 6 Inspection of the Effect of Gene Editing by Introducing Specific RNA Produced by In Vitro Transcription into a Target System with/without Binding of ORF1p and/or ORF2p Outside the Target System

The GALT gene encodes galactose-1-phosphate uridyltransferase (GALT), and its mutation can lead to human type I galactosemia.

Construct a initiating ORF2p splicing and reverse transcription functional structure, where the RNA of this functional structure binds to its complementary sequence on the genome to form an “Ω” structure.

The downstream sequence (downstream sequence of target site) of the sequence t be inserted of the GALT gene with the sequence t be inserted as shown in Seq ID No.25 in Embodiment 4 was selected as the 5′ part of the “Ω” structure (left leg), as shown in Seq ID No.38:

ATGCATGGGCCTCAGTCACAGAGGAGCTGGGTGCCCAGTACCCTTGGGT
GCAGGTTTGTGAGGTCGCCCCTTCCCCTGGATGGGCAGGGAGGGGGTGA
TGAAGCTTTGGTTCTGGGGAGTAACATTTCTGTTTCCACAGGGTGTGGT
CAGGAGGGAGTTGACTTGGTGTCTTTTGGCTAACAGAGCTCCGTATCCC
TATCTGATAGATCTTTG.

The 3′ part (right leg) of the “Ω” structure is composed of downstream sequence immediately following the sequence shown in Seq ID No.38 (downstream sequence of target site) on the genome, as shown in Seq ID No.39:

AAAACAAAGGTGCCATGATGGGCTGTTCTAACCCCCACCCCCACTGCCA
GGTAAGGGTGTCAGGGGCTCCAGTGGGTTTCTTGGCTGAGTCTGAGCCA
GCACT;

The circular part of the “Ω” structure is composed of randomly generated sequence, as shown in Seq ID No.40:

CTGACCATGCTTATACGGACTATCGATTAG_{○}

Connect the circular part of the “Ω” structure shown in Seq ID No.40 and the right leg structure of “Ω” structure shown in Seq ID No.39 to the downstream of the sequence shown in Seq ID No.25, and add the T7 promoter sequence shown in Seq ID No.11 upstream of the sequence shown in Seq ID No.25 to construct the sequence shown in Seq ID No.41:

TAATACGACTCACTATAGGGGTTCGGCCCTGCCCGTAGCACAGCCAAGCCCTA
CCTCTCGGTTATCTTTTCTCCCGTCACCACCCAGTAAGGTCATGTGCTTCCACCCCT
GGTCGGATGTAACGCTGCCACTCATGTCGGTCCCTGAGATCCGGGCTGTTGTTGTG
ACTACTGAGATTACTTTGACATGTCCCACTTATTAATATCACCTTAAGTTTGGGTTC
GATTAATATTATGTAACCTGTGAACGAGATAAGATTCTAGAGATTTAATCGAACCTT
AATTCTGATTCGGTTATGTCAAAAGGTGTCTTGAATGCATGGGCCTCAGTCACAGA
GGAGCTGGGTGCCCAGTACCCTTGGGTGCAGGTTTGTGAGGTCGCCCCTTCCCCT
GGATGGGCAGGGAGGGGGTGATGAAGCTTTGGTTCTGGGGAGTAACATTTCTGTT
TCCACAGGGTGTGGTCAGGAGGGAGTTGACTTGGTGTCTTTTGGCTAACAGAGCT

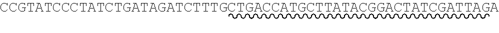

AAACAAAGGTGCCATGATGGGCTGTTCTAACCCCCACCCCCACTGCCAGGTAAG
GGTGTCAGGGGCTCCAGTGGGTTTCTTGGCTGAGTCTGAGCCAGCACT

Wherein the italicized bold represents the T7 promoter sequence as shown in Seq ID No.11, the wavy line sequence is the circular part of the “Ω” structure as shown in Seq ID No.40, the downstream of the wavy line sequence is the right leg structure of the “Ω” structure as shown in Seq ID No.39, the left leg structure of the “Ω” structure shown in Seq ID No.38 (downstream sequence of the target site) is located between the upstream of the wavy line sequence and the underline. The sequence shown in Seq ID No.41 was obtained through chemical synthesis and named as the precursor DNA of RNA+initiating ORF2p splicing and reverse transcription functional structure.

According to the instructions of the MEGAscript™ T7 Transcription Kit, the linear precursor DNA of RNA+initiating ORF2p splicing and reverse transcription functional structure were transcribed to obtain corresponding RNA. Then, the residual DNA was degraded by DNase from the kit and resuspended in RNase free water. The RNA concentration was measured by UV spectrophotometer, and the concentration of RNA+initiating ORF2p splicing and reverse transcription functional structure solution was further configured to 100 ng/μL by adding RNase free water.

The RNA+initiating ORF2p splicing and reverse transcription functional structure obtained from the above transcription belongs to the RNA framework structure in FIG. 6, where the “Ω” structure is formed by the initiating ORF2p splicing and reverse transcription functional structure.

Resuspend hLRE1-ORF1p and hLRE1-ORF2p prepared in Embodiment 1 with Opti MEM solution pre added with 1 U/μL RNA enzyme inhibitor, respectively, into 500 ng/μL of hLRE1-ORF1p solution or hLRE1-ORF2p solution.

Prepare RNP of RNA+initiating ORF2p splicing and reverse transcription functional structure bound with ORF1p and/or ORF2p: RNA+initiating ORF2p splicing and reverse transcription functional structure+hLRE1-ORF1p+hLRE1-ORF2p. Follow the steps below.

Configure the reaction system as shown in Table 28:

TABLE 28
Reaction System

| Component | Addition Amount | |
|---|---|---|
| RNA + initiating ORF2p splicing and reverse | 21.0 | μL |
| transcription functional structure solution | | |
| hLRE1-ORF2p solution | 5.5 | μL |

| | |
|---|---|
| Opti-MEM ™ I medium | Make up to 50 μL |

Mix the components gently and incubate the reaction system at room temperature (25° C.) for 10 minutes to obtain RNA+initiating ORF2p splicing and reverse transcription functional structure+hLRE1-ORF2p solution.

Then, mix the RNA+initiating ORF2p splicing and reverse transcription functional structure+hLRE1-ORF2p solution according to the system shown in Table 29.

TABLE 29
Reaction System

| Component | Addition Amount | |
|---|---|---|
| RNA + initiating ORF2p splicing and | 50.0 | μL |
| reverse transcription functional | | |
| structure + hLRE1-ORF2p solution | | |
| hLRE1-ORF1p solution | 3.0 | μL |

| | |
|---|---|
| Opti-MEM ™ I medium | Make up to 75 μL |

After gently mixing each component, the reaction system was incubated at room temperature (25° C.) for 10 minutes to obtain RNA+initiating ORF2p splicing and reverse transcription functional structure+hLRE1-ORF1p+hLRE1-ORF2p solution.

Mix RNA+initiating ORF2p splicing and reverse transcription functional structure+hLRE1-ORF1p+hLRE1-ORF2p solution in equal volume ratio with the transfection solution system prepared in Table 7. Gently mix and incubate at room temperature (25° C.) for 20 minutes to obtain RNA+initiating ORF2p splicing and reverse transcription functional structure+hLRE1-ORF1p+hLRE1-ORF2p-liposome complex.

### Transfection

Firstly, human glioma U251 cells were passaged and placed on a 24 well plate, cultured in complete medium. The next day after passaging, when human glioma U251 cells grew to about 60% confluence, replacing the medium with Opti-MEM™ I medium. According to the instructions of the RNAiMAX transfection reagent, RNA+initiating ORF2p splicing and reverse transcription functional structure+hLRE1-ORF1p+hLRE1-ORF2p-liposome complex was added to human glioma U251 cells for transfection, with three parallels for each group. Continue to culture cells until they grew to about 90% confluence before passaging. After passaging, repeat transfection once (following the previous steps). After the cells grew again to about 90% confluence, proceeded with subsequent operations.

### Experimental Grouping

Take the control group in Embodiment 4 as the control group, and RNA+initiating ORF2p splicing and reverse transcription functional structure+hLRE1-ORF1p+hLRE1-ORF2p as the experimental group.

Extraction of transfected cell DNA in each group: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the copy number of GAPDH gene is stable, the GAPDH gene was used as a reference gene.

The upstream primer sequence for detecting the GAPDH gene is shown in Seq ID No.19. The downstream primer sequence is shown in Seq ID No.20.

A primer pair 2 was designed, wherein an upstream primer sequence of the primer pair 2 is shown as Seq ID No.31: 5′-CCCCAGTACGATAGCACC-3′; and the downstream primer sequence is shown in Seq ID No.32: 5′-GACATAACCGAATCAGAATT-3′. The upstream primer sequence of primer pair 2 is located in the complete GALT gene, further upstream of the upstream sequence of the insertion site (target site) used on the RNA prepared, not in the RNA prepared, but only in the genome, and the downstream primer sequence of primer pair 2 is located on the exogenous sequence to be inserted (the sequence to be inserted).

The primers were all obtained through chemical synthesis.

The qPCR reaction system is shown in Table 30.

TABLE 30
qPCR reaction system

| Component | Addition Amount | |
|---|---|---|
| Upstream primer (10 μM) | 1.5 | μL |
| Downstream primer (10 μM) | 1.5 | μL |
| 2 × SuperReal PreMix Plus | 25 | μL |
| Cellular DNA template (25 ng/μL) | 4 | μL |

| | |
|---|---|
| ddH₂O without RNase | Make up to 50 μL |

The cellular DNA template was DNA extracted from the cells of experimental group after transfection.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 2: pre-denaturation at 95° C. for 15 mim; (denaturation at 95° C. for 10 s, annealing at 46° C. for 20 s, and extension at 72° C. for 20 s) 40 cycles. The GAPDH primers were reacted under the same conditions.

The exponential growth phases in the amplification curves of GAPDH and the detection of the insertion of the sequence to be inserted were observed and were confirmed to be approximately parallel, the obtained data were analyzed by the 2^{-ΔΔCt} method, and the results are shown in Table 31. The PCR products were verified to be correct by sequencing.

TABLE 31
Results of primer pair 2 (n = 3, x̅ ± s)

| | | | | ΔΔCt (mean | |
|---|---|---|---|---|---|
| | | | | ΔCt of the | Relative |
| | | Insertion of | | experimental | amount |
| | | the sequence | | group − mean | of copy |
| | | to be | | ΔCt of the | number |
| Ct value | GAPDH | inserted | ΔCt | control group) | (2^{-ΔΔCt}) |
| Control group | 26.27 | N/A | 13.73 | — | — |
| plate 1 | | | | | |
| Control group | 26.00 | N/A | 14.00 | — | — |
| plate 2 | | | | | |
| Control group | 26.44 | N/A | 13.56 | — | — |
| plate 3 | | | | | |
| Control group | | | 13.76 ± | 0.00 ± | 1.00 |
| mean | | | 0.18 | 0.18 | (0.88-1.13) |
| Experimental | 26.08 | 38.77 | 12.69 | — | — |
| group plate 1 | | | | | |
| Experimental | 25.86 | 38.12 | 12.26 | — | — |
| group plate 2 | | | | | |
| Experimental | 26.48 | 38.82 | 12.34 | — | — |
| group plate 3 | | | | | |
| Experimental | | | 12.43 ± | −1.33 ± | 2.51 |
| group mean | | | 0.19 | 0.19 | (2.20-2.87) |

According to Table 31, compared with the control group, the relative copy number of the experimental group was significantly higher than that of the control group, with statistical significance (P<0.05), indicating that even if the complete or partial short interspersed element or long interspersed element sequence is not connected, As long as the 3′ part of the RNA framework containing upstream sequence of target site, downstream sequence of target site, and the sequence to be inserted can form a specific secondary structure such as an “Ω”-shaped structure and can recruit and bind to ORF2p (such as through poly A sequence), the corresponding gene editing objective can be achieved.

### Embodiment 7 Inspection the Efficiency of Modifying Gene Loci on the Genome after Transcription and Synthesis of RNA, ORF1p, and ORF2p within the Eukaryotic Target System

The PAH gene encodes and expresses phenylalanine hydroxylase, which is a pathogenic gene for phenylketonuria.

Select a 250 bp sequence from the gene PAH in the human genome, as shown in Seq ID No.42:
AAAATGCCACTGAGAACTCTCTTAAGACTACCTTTCTCCAAATGGTGCCCTT CACTCAAGCCTGTGGTTTTGGTCTTAGGAACTTTGCTGCCACAATACCTCGGCCC TTCTCAGTTCgCTACGACCCATACACCCAAAGGATTGAGGTCTTGGACAATACCC AGCAGCTTAAGATTTTGGCTGATTCCATTAACAGTAAGTAATTTACACCTTACGA GGCCACTCGGTTTCTCAGTAATCGAAGACTGTC, wherein the lowercase letter represents the base to be modified.

The sequence after modifying the base from G to C is shown as Seq ID No.43:
AAAATGCCACTGAGAACTCTCTTAAGACTACCTTTCTCCAAATGGTGCCCTT CACTCAAGCCTGTGGTTTTGGTCTTAGGAACTTTGCTGCCACAATACCTCGGCCC TTCTCAGTTCcCTACGACCCATACACCCAAAGGATTGAGGTCTTGGACAATACCC AGCAGCTTAAGATTTTGGCTGATTCCATTAACAGTAAGTAATTTACACCTTACGA GGCCACTCGGTTTCTCAGTAATCGAAGACTGTC, wherein, the lowercase letter represents the modified base.

Take the pre modified sequence Seq ID No.42 as the upstream sequence of target site, the modified sequence Seq ID No.43 as the sequence to be inserted, and a 200 bp sequence located downstream of Seq ID No.42 on the gene and next to Seq ID No.42 as the downstream sequence of target site, resulting in the sequence shown in Seq ID No.44:

AAAATGCCACTGAGAACTCTCTTAAGACTACCTTTCTCCAAATGGTGCC
CTTCACTCAAGCCTGTGGTTTTGGTCTTAGGAACTTTGCTGCCACAATA
CCTCGGCCCTTCTCAGTTCGCTACGACCCATACACCCAAAGGATTGAGG
TCTTGGACAATACCCAGCAGCTTAAGATTTTGGCTGATTCCATTAACAG
TAAGTAATTTACACCTTACGAGGCCACTCGGTTTCTCAGTAATCGAAGA
CTGTCAAAATGCCACTGAGAACTCTCTTAAGACTACCTTTCTCCAAATG
GTGCCCTTCACTCAAGCCTGTGGTTTTGGTCTTAGGAACTTTGCTGCCA
CAATACCTCGGCCCTTCTCAGTTCCCTACGACCCATACACCCAAAGGAT
TGAGGTCTTGGACAATACCCAGCAGCTTAAGATTTTGGCTGATTCCATT
AACAGTAAGTAATTTACACCTTACGAGGCCACTCGGTTTCTCAGTAATC
GAAGACTGTCTTTCCCTACCATCGCCATAGGAAAAATAATAAATTTATT
GAAATATTTAATTAAGGAGAAAAGCACCTCCATGTAAGCCATGGGTTCA
TTGATGGAGAAGAACTTGACAAAAAGGTCAGAATTACCCTTGTGTCCTT
TTTCCTTTGACCTTCCTAGATTCCACTCCACCTCCTACCATCATTCCAC
CTTTCCACACTTGG,

wherein, the underline represents the sequence to be inserted, Seq ID No.43. Its upstream is the upstream sequence of target site, Seq ID No.42, and downstream is the downstream sequence of target site. This sequence is named the PAH base replacement sequence.

Adding a partial Alu sequence downstream of the PAH base replacement sequence Seq ID No.44 to obtain the sequence shown in Seq ID No.45:

AAAATGCCACTGAGAACTCTCTTAAGACTACCTTTCTCCAAATGGTGCCCTT
CACTCAAGCCTGTGGTTTTGGTCTTAGGAACTTTGCTGCCACAATACCTCGGCCC
TTCTCAGTTCGCTACGACCCATACACCCAAAGGATTGAGGTCTTGGACAATACCC
AGCAGCTTAAGATTTTGGCTGATTCCATTAACAGTAAGTAATTTACACCTTACGA
GGCCACTCGGTTTCTCAGTAATCGAAGACTGTCAAAATGCCACTGAGAACTCTCT
TAAGACTACCTTTCTCCAAATGGTGCCCTTCACTCAAGCCTGTGGTTTTGGTCTTA
GGAACTTTGCTGCCACAATACCTCGGCCCTTCTCAGTTCCCTACGACCCATACAC
CCAAAGGATTGAGGTCTTGGACAATACCCAGCAGCTTAAGATTTTGGCTGATTCC
ATTAACAGTAAGTAATTTACACCTTACGAGGCCACTCGGTTTCTCAGTAATCGAA
GACTGTCTTTCCCTACCATCGCCATAGGAAAAATAATAAATTTATTGAAATATTT
AATTAAGGAGAAAAGCACCTCCATGTAAGCCATGGGTTCATTGATGGAGAAGAA
CTTGACAAAAAGGTCAGAATTACCCTTGTGTCCTTTTTCCTTTGACCTTCCTAGAT

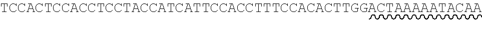

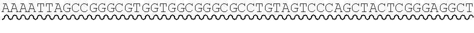

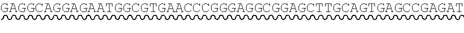

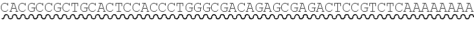

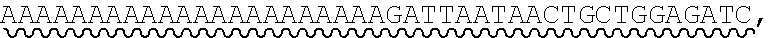

wherein, the underline represents the sequence to be inserted Seq ID No.43, its upstream is the upstream sequence of target site Seq ID No.42, the wavy line represents the partial Alu sequence shown in Seq ID No.2, the downstream sequence of target site is between the sequence to be inserted and the partial Alu sequence, which is named PAH base replacement sequence framework+partial Alu sequence.

The sequence shown in Seq ID No.45 was chemically synthesized and constructed into the pcDNA3.1(+)eGFP vector through homologous recombination linkage, so that the sequence is directly connected downstream of the CMV promoter in the vector, and there are no other sequences between the sequence and the CMV promoter. The plasmid obtained was named pcDNA3.1(+)eGFP+PAH base replacement sequence framework+partial Alu sequence.

The specific steps are:

1. Design primers to amplify the sequence shown in Seq ID No.45, where the forward primer sequence is as shown in Seq ID No.46: 5′-CTATATAAGCAGAGCTAAAATGCCACTGAGAA-3′, and the reverse primer sequence is as shown in Seq ID No.16: 5′-CTCTAGTTAGCCAGAGGATCTCCAGCAGTTAT-3′. Perform PCR amplification on the sequence shown in Seq ID No.45, and the reaction system is shown in Table 32:

TABLE 32
Reaction System

| Component | Addition Amount | |
|---|---|---|
| 2 × KOD PCR Master mix | 25 | μL |
| forward primer (10 μM) | 1 | μL |
| reverse primer (10 μM) | 1 | μL |
| synthesized sequence (10 pg-200 ng) | ~1 | μL |
| ddH₂O | ~22 | μL |

The amplification conditions are: 94° C. for 2 minutes; Perform 40 cycles at 98° C. for 10 seconds, 58° C. for 10 seconds, and 68° C. for 2 seconds; 68° C. for 5 minutes.

The amplification product was obtained by gel electrophoresis, extraction and purification with conventional methods. The two sides of the synthesized sequence in the amplification product were added with the sequence homologous to pcDNA3.1(+)eGFP vector.

2. Design PCR primers to amplify the pcDNA3.1(+)eGFP vector, with a forward primer as shown in Seq ID No.17: 5′-AATAACTGCTGGAGATCCTCTGGCTAACTAGAG-3′ and a reverse primer sequence as shown in Seq ID No.47: 5′-GTTCTCAGTGGCATTTTAGCTCTGCTTATATAG-3′. Perform PCR amplification on the pcDNA3.1(+)eGFP vector, and the reaction system is shown in Table 33:

TABLE 33
Reaction System

| Component | Addition Amount | |
|---|---|---|
| 2 × KOD PCR Master mix | 25 | μL |
| forward primer (10 μM) | 1 | μL |
| reverse primer (10 μM) | 1 | μL |
| pcDNA3.1(+)eGFP plasmid template (10 pg-200 ng) | ~1 | μL |
| ddH₂O | ~22 | μL |

The amplification conditions are: 94° C. for 2 minutes; Perform 40 cycles at 98° C. for 10 seconds, 58° C. for 10 seconds, and 68° C. for 6 seconds; 68° C. for 5 minutes.

The pcDNA3.1(+)eGFP plasmid vector was obtained by gel electrophoresis, extraction and purification with conventional methods. The two sides of the plasmid vector had homologous sequences with the synthesized sequence.

3. Use a one-step rapid cloning kit to connect the amplified product to the amplified pcDNA3.1(+)eGFP vector. Follow the instructions in the kit for specific steps, and the reaction system is shown in Table 11.

4. Transfect the recombinant product into competent cells (DH5α), pick the colonies on plate and sequence. After sequencing, extract the plasmid to obtain pcDNA3.1(+)eGFP-PAH base replacement sequence framework+partial Alu sequence.

Co transfect the constructed vector pcDNA3.1(+)eGFP-PAH base replacement sequence framework+partial Alu sequence with plasmids pBS-L1PA1-CH-mneo expressing ORF1p and ORF2p into Hela cells. The experimental group consists of group co transfected with pcDNA3.1(+)eGFP-PAH base replacement sequence framework+partial Alu sequence and pBS-L1PA1-CH-mneo, and the control group consists of group co transfected with plasmids pBS-L1PA1-CH-mneo and pcDNA3.1(+)eGFP. Each group has 3 parallels, each consisting of a 24 well plate cultured with Hela cells.

The transfection experiment steps of the control group are the same as those of the control group in Embodiment 2.

The co transfection steps of the experimental group are: Hela cells were passaged and spread into 24-well plates. On the next day of passage, when the cells grew to about 60% confluence, transfection was performed using Entranster-H4000 transfection reagent. For transfection of each plate of cells, two plasmids (pcDNA3.1(+)eGFP-PAH base replacement sequence framework+partial Alu sequence and pBS-L1PA1-CH-mneo) were co transfected, with each plasmid transfected 19.2 μg, for a total of 38.4 μg. The required plasmids were diluted with 600 μL serum-free DMEM and mixed thoroughly. At the same time, 48 μL of Entranster-H4000 reagent was diluted with 600 μL of serum-free DMEM, and after fully mixed, it was allowed to stand for 5 min at room temperature. Then the prepared two liquids were mixed and fully mixed, and allowed to stand for 15 min at room temperature to prepare the transfection complex. The transfection complex was added to 24-well plate with Hela cells, in which contained 0.5 ml Opti-MEM medium per well, for transfection. After the cells grew to about 90% confluence, they were passaged, and the above operations were repeated after passage. After the cells grew to about 90% confluence again, samples were taken for subsequent operations.

The co transfection steps of the control group are: Hela cells were passaged and spread into 24-well plates. On the next day of passage, when the cells grew to about 60% confluence, transfection was performed using Entranster-H4000 transfection reagent. For transfection of each plate of cells, two plasmids (pBS-L1PA1-CH-mneo and pcDNA3.1(+)eGFP) were co transfected, with each plasmid transfected 19.2 μg, for a total of 38.4 μg. The required plasmids were diluted with 600 μL serum-free DMEM and mixed thoroughly. At the same time, 48 μL of Entranster-H4000 reagent was diluted with 600 μL of serum-free DMEM, and after fully mixed, it was allowed to stand for 5 min at room temperature. Then the prepared two liquids were mixed and fully mixed, and allowed to stand for 15 min at room temperature to prepare the transfection complex. The transfection complex was added to 24-well plate with Hela cells, in which contained 0.5 ml Opti-MEM medium per well, for transfection. After the cells grew to about 90% confluence, they were passaged, and the above operations were repeated after passage. After the cells grew to about 90% confluence again, samples were taken for subsequent operations.

Extraction of transfected cell DNA: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the copy number of GAPDH gene is stable, the GAPDH gene was used as a reference gene.

The upstream primer sequence for detecting the GAPDH gene is shown in Seq ID No.19 as follows: 5′-CACTGCCACCCAGAAGACTG-3′. The downstream primer sequence is shown in Seq ID No.20: 5′-CCTGCTTCACCACCTTCTTG-3′.

A primer pair 3 was designed, wherein an upstream primer sequence of the primer pair 3 is shown as Seq ID No.48: 5′-AGGGAGGTGTCCGTGTTC-3′; and the downstream primer sequence is shown in Seq ID No.49: 5′-GGGTGTATGGGTCGTAGC-3′. The upstream primer sequence of primer pair 3 is located in the complete PAH gene, further upstream of the upstream sequence of insertion site (target site), does not exist in the sequence of the constructed vector, only exists in the cell genome, and the downstream primer sequence of primer pair 3 is located on the sequence to be inserted, its 3′ end base matches the unmodified base on the genome, so if the selected base site on the genome is modified, the PCR product will decrease.

The primers were all obtained through chemical synthesis.

The qPCR reaction system is shown in Table 34.

TABLE 34
qPCR reaction system

| Component | Addition Amount | |
|---|---|---|
| Upstream primer (10 μM) | 1.5 | μL |
| Downstream primer (10 μM) | 1.5 | μL |
| 2 × SuperReal Premix Plus | 25 | μL |
| Cellular DNA template (25 ng/μL) | 4 | μL |

| | |
|---|---|
| ddH₂O without RNase | Make up to 50 μL |

The cellular DNA template was DNA extracted from the aforementioned 2 groups after transfection.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 3: pre-denaturation at 95° C. for 15 min; (denaturation at 95° C. for 10 s, annealing at 48° C. for 20 s, and extension at 72° C. for 20 s) 40 cycles. The GAPDH primers were reacted under the same conditions.

The exponential growth phases in the amplification curves of GAPDH and the detection of the original site of the site to be modified were observed and were confirmed to be approximately parallel, the obtained data were analyzed by the 2^{-ΔΔCt} method, and the results are shown in Table 35. The PCR products were verified to be correct by sequencing.

TABLE 35
Results of primer pair 3 (n = 3, x̅ ± s)

| | | | | ΔΔCt (mean | |
|---|---|---|---|---|---|
| | | Original | | ΔCt of the | |
| | | site of | | experimental | Relative |
| | | the site | | group − mean | amount of |
| | | to be | | ΔCt of the | copy number |
| Ct value | GAPDH | modified | ΔCt | control group) | (2^{-ΔΔCt}) |
| Control group | 25.96 | 25.73 | −0.23 | — | — |
| plate 1 | | | | | |
| Control group | 26.08 | 26.16 | 0.08 | — | — |
| plate 2 | | | | | |
| Control group | 26.36 | 26.54 | 0.18 | — | — |
| plate 3 | | | | | |
| Control group | | | 0.01 ± | 0.00 ± | 1.00 |
| mean | | | 0.17 | 0.17 | (0.89-1.13) |
| Experimental | 25.62 | 26.89 | 1.27 | — | — |
| group plate 1 | | | | | |
| Experimental | 26.00 | 27.17 | 1.17 | — | — |
| group plate 2 | | | | | |
| Experimental | 25.74 | 27.29 | 1.55 | — | — |
| group plate 3 | | | | | |
| Experimental | | | 1.33 ± | 1.32 ± | 0.40 |
| group mean | | | 0.16 | 0.16 | (0.36-0.45) |

According to Table 35, compared with the control group, the relative copy number of the experimental group was significantly higher than that of the control group, with statistical significance (P<0.05), indicating that the present invention can achieve the goal of replacing specific loci on the genome.

At the same time, it indicates that genomic sequence replacement, site deletion, site addition, sequence addition, and sequence deletion, which are also based on homologous recombination on the genome after specific sequence insertion just like the replacement of specific loci on the genome, are also feasible.

Due to the fact that direct plasmid transfection can achieve the goal of site replacement on the genome, according to other Embodiments of the present invention, it can also be achieved by in vitro transcription of the corresponding RNA, and transfection with or without ORF1p and/or ORF2p.

Due to the fact that both eukaryotic and prokaryotic systems can express RNA and have homologous recombination ability, which is similar to the relevant working mechanism associated with the present invention, sequence insertion on the genome can be achieved. However, the presence of splicing mechanisms in eukaryotic systems may interfere with the synthesis of specific RNA required and cause inconvenience in industrial production. In addition, sequence replacement, site deletion, site addition, sequence addition, sequence deletion, and site replacement on the genome are achieved by cells themselves through homologous recombination mechanisms after inserting corresponding sequences by the present invention. Therefore, genome modification operations such as sequence replacement, site deletion, site addition, sequence addition, sequence deletion, and site replacement on eukaryotic systems are feasible, which means that corresponding operations on prokaryotic systems are also feasible.

### Embodiment 8 Inspection the Efficiency of Administering RNA Framework and Short Interspersed Element RNA Separately

Connect the T7 promoter sequence shown in Seq ID No.11 with the Alu sequence shown in Seq ID No.1 to obtain the sequence shown in Seq ID No.50:
TAATACGACTCACTATAGGGCCGGGCGCGGTGGCTCACGCCTGTAATCCCAG CACTTTGGGAGGCCGAGGCGGGCGGATCACGAGGTCAGGAGATCGAGACCATCC CGGCTAAAACGGTGAAACCCCGTCTCTACTAAAAATACAAAAAATTAGCCGGGC GTGGTGGCGGGCGCCTGTAGTCCCAGCTACTCGGGAGGCTGAGGCAGGAGAATG GCGTGAACCCGGGAGGCGGAGCTTGCAGTGAGCCGAGATCACGCCGCTGCACTC CACCCTGGGCGACAGAGCGAGACTCCGTCTCAAAAAAAAAAAAAAAAAAAAAA AAAAAAAAGATTAATAACTGCTGGAGATC, wherein the bold part in italics represents the T7 promoter sequence, and downstream of the T7 promoter sequence is the Alu sequence, the sequence was obtained through chemical synthesis and named as the DNA of Alu-RNA expression.

According to the instructions of the MEGAscript™ T7 Transcription Kit, the linear DNA of Alu-RNA was transcribed to obtain corresponding Alu-RNA. Then, the residual DNA was degraded by DNase from the kit and resuspended in RNase free water. The RNA concentration was measured by UV spectrophotometer, and the concentration of the solution was further configured to 100 ng/μL by adding RNase free water.

Prepare Alu-liposome complex, and the reaction system is shown in Table 36:

TABLE 36
Reaction System

| Component | Addition Amount |
|---|---|
| Alu solution | 21.0 μL |
| Opti-MEM ™ I medium | Make up to 75 μL |

Gently mix the components and incubate the reaction system at room temperature (25° C.) for 10 minutes. Then, add the transfection solution as shown in Table 7 in equal volume ratio. Gently mix and incubate at room temperature (25° C.) for 20 minutes to obtain the Alu-liposome complex.

The control group in embodiment 2 was used as the control group, and the RNA+hLRE1-ORF1p+hLRE1-ORF2p-liposome complex in embodiment 2 and Alu-liposome complex were co transfected (experimental group) into Hela cells according to the method in Embodiment 2 (Alu-liposome complex and RNA+hLRE1-ORF1p+hLRE1-ORF2p-liposome complex were co transfected in equal amounts), with three parallels in each group, each consisting of a 24 well plate cultured with Hela cells.

The Alu transcribed above and the RNA in embodiment 2 form the isolated transcriptional structure shown in FIG. 14.

Extraction of transfected cell DNA in each group: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the copy number of GAPDH gene is stable, the GAPDH gene was used as a reference gene.

The upstream primer sequence for detecting the GAPDH gene is shown in Seq ID No.19 as follows: 5′-CACTGCCACCCAGAAGACTG-3′. The downstream primer sequence is shown in Seq ID No.20: 5′-CCTGCTTCACCACCTTCTTG-3′.

A primer pair 1 was designed, wherein an upstream primer sequence of the primer pair 1 is shown as Seq ID No.21: 5′-GACTTATCCATGTGCCTGTT-3′; and the downstream primer sequence is shown in Seq ID No.22: 5′-TTGGCTACGAAATGCTTG-3′. The upstream primer sequence of primer pair 1 is located in the complete Lman1 gene, further upstream of the upstream sequence of the insertion site (target site) used on the RNA prepared, not in the RNA prepared, but only in the genome, and the downstream primer sequence of primer pair 1 is located on the exogenous sequence to be inserted (the sequence to be inserted).

The primers were all obtained through chemical synthesis.

The qPCR reaction system is shown in Table 37.

TABLE 37
qPCR reaction system

| Component | Addition Amount | |
|---|---|---|
| Upstream primer (10 μM) | 1.5 | μL |
| Downstream primer (10 μM) | 1.5 | μL |
| 2 × SuperReal PreMix Plus | 25 | μL |
| Cellular DNA template (25 ng/μL) | 4 | μL |

| | |
|---|---|
| ddH₂O without RNase | Make up to 50 μL |

The cell DNA template was extracted from the transfected cells in the above 2 groups.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 1: pre-denaturation at 95° C. for 15 min; (denaturation at 95° C. for 10 s, annealing at 49° C. for 20 s, and extension at 72° C. for 20 s) 40 cycles. The GAPDH primers were reacted under the same conditions.

The exponential growth phases in the amplification curves of GAPDH and the detection of the insertion of the sequence to be inserted were observed and were confirmed to be approximately parallel, the obtained data were analyzed by the 2^{-ΔΔCt} method, and the results are shown in Table 38. The PCR products were verified to be correct by sequencing.

TABLE 38
Results of primer pair 1 (n = 3, x̅ ± s)

| | | | | ΔΔCt (mean | |
|---|---|---|---|---|---|
| | | | | ΔCt of the | |
| | | Insertion | | experimental | Relative |
| | | of sequence | | group − mean | amount of |
| | | to be | | ΔCt of the | copy number |
| Ct value | GAPDH | inserted | ΔCt | control group 1) | (2^{-ΔΔCt}) |
| Control group 1 | 26.23 | N/A | 13.77 | — | — |
| plate 1 | | | | | |
| Control group 1 | 26.14 | N/A | 13.86 | — | — |
| plate 2 | | | | | |
| Control group 1 | 25.80 | N/A | 14.20 | — | — |
| plate 3 | | | | | |
| Control group 1 | | | 13.94 ± | 0.00 ± | 1.00 |
| mean | | | 0.19 | 0.19 | (0.88-1.14) |
| Experimental | 25.98 | 35.84 | 9.86 | — | — |
| group 1 plate 1 | | | | | |
| Experimental | 26.19 | 36.53 | 10.34 | — | — |
| group 1 plate 2 | | | | | |
| Experimental | 25.89 | 36.08 | 10.19 | — | — |
| group 1 plate 3 | | | | | |
| Experimental | | | 10.13 ± | −3.81 ± | 14.03 |
| group 1 mean | | | 0.20 | 0.20 | (12.21-16.11) |

As shown in Table 38, compared with the control group, the relative copy number of the experimental group was significantly higher than that of the control group, with statistical significance (P<0.05), indicating that RNA frameworks containing upstream sequence of target site, downstream sequence of target site, and sequences to be inserted, as well as corresponding transcriptional product RNAs of short interspersed element, partial short interspersed element, long interspersed element, and/or partial long interspersed element, can be given receiving systems at different positions of the same vector or on different vectors separately to achieve the insertion of the specified sequence into the specified gene target site or other gene editing purposes such as genome site substitution, sequence substitution, site deletion, site addition, sequence addition, and sequence deletion, etc. At the same time, DNA vectors that can express RNA frameworks containing upstream sequence of target site, downstream sequence of target site, and sequences to be inserted, as well as DNA vectors that can express short interspersed element, partial short interspersed element, long interspersed element, and/or partial long interspersed element, can be provided to the receiving system separately at different positions of the same vector or on different vectors, and can also achieve the corresponding gene editing purpose. At the same time, RNA frameworks that contain upstream sequence of target site, downstream sequence of target site, and sequences to be inserted, as well as DNA vectors that can express short interspersed element, partial short interspersed element, long interspersed element, and/or partial long interspersed element, can be provided to the receiving system separately at different positions of the same vector or on different vectors, and can also achieve the corresponding gene editing purpose. At the same time, DNA vectors that can express RNA frameworks containing upstream sequence of target site, downstream sequence of target site, and sequences to be inserted, as well as corresponding transcriptional product RNA of short interspersed element, partial short interspersed element, long interspersed element, and/or partial long interspersed element, can be provided to the receiving system separately at different positions of the same vector or on different vectors, and can also achieve the corresponding gene editing purpose.

### Embodiment 9 Gene Editing Effects of ORF1 and ORF2p in Human LRE2 and ORF1 and ORF2p in Mouse L1

The ORF1p and ORF2p used in Embodiments 2-8 are derived from LRE1 in human L1. In this embodiment, it was verified whether ORF1p (hLRE2-ORF1p) and ORF2p (hLRE2-ORF2p) in human LRE2, as well as ORF1p (mORF1p) and ORF2p (mORF2p) in mouse L1 can still play a gene editing role by replacing the previous ORF1p and ORF2p in LRE1.

Combine the RNA+partial Alu solution prepared in Embodiment 2 with the hLRE2-ORF1p, hLRE2-ORF2p, mORF1p, and mORF2p prepared in Embodiment 1 to prepare RNA+partial Alu+hLRE2-ORF1p+hLRE2-ORF2p, and RNA+partial Alu+mORF1p+mORF2p, respectively, using the preparation method of RNA+partial Alu+hLRE1-ORF1p+hLRE1-ORF2p in Embodiment 2.

### Experimental Grouping:

It was divided into three groups: the control group in Embodiment 2 is used as the control group in this Embodiment, the group transfected with RNA+partial Alu+hLRE2-ORF1p+hLRE2-ORF2p was experimental group 1, and the group transfected with RNA+partial Alu+mORF1p+mORF2p was experimental group 2. Each group had three parallels, each of which was a 24 well plate cultured with Hela cells.

Extraction of transfected cell DNA in each group: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the copy number of GAPDH gene is stable, the GAPDH gene was used as a reference gene.

The upstream primer sequence for detecting the GAPDH gene is shown in Seq ID No.19 as follows: 5′-CACTGCCACCCAGAAGACTG-3′. The downstream primer sequence is shown in Seq ID No.20: 5′-CCTGCTTCACCACCTTCTTG-3′.

A primer pair 1 was designed, wherein an upstream primer sequence of the primer pair 1 is shown as Seq ID No.21: 5′-GACTTATCCATGTGCCTGTT-3′; and the downstream primer sequence is shown in Seq ID No.22: 5′-TTGGCTACGAAATGCTTG-3′. The upstream primer sequence of primer pair 1 is located in the complete Lman1 gene, further upstream of the upstream sequence of the insertion site (target site) used on the RNA prepared, not in the RNA prepared, but only in the genome, and the downstream primer sequence of primer pair 1 is located on the exogenous sequence to be inserted (the sequence to be inserted).

The primers were all obtained through chemical synthesis.

The qPCR reaction system is shown in Table 39.

TABLE 39
qPCR reaction system

| Component | Addition Amount | |
|---|---|---|
| Upstream primer (10 μM) | 1.5 | μL |
| Downstream primer (10 μM) | 1.5 | μL |
| 2 × SuperReal PreMix Plus | 25 | μL |
| Cellular DNA template (25 ng/μL) | 4 | μL |

| | |
|---|---|
| ddH₂O without RNase | Make up to 50 μL |

The cell DNA template was extracted from the transfected cells in the above each group.

The reaction system was prepared on ice, put the lid of the reaction tube on after preparation, mixed gently, and centrifuged briefly to ensure that all the components were at the bottom of the tube. Each 24-well plate cell sample was repeated 3 times simultaneously.

qPCR Reaction Cycle:

Primer pair 1: pre-denaturation at 95° C. for 15 min; (denaturation at 95° C. for 10 s, annealing at 49° C. for 20 s, and extension at 72° C. for 20 s) 40 cycles. The GAPDH primers were reacted under the same conditions.

The exponential growth phases in the amplification curves of GAPDH and the detection of the insertion of the sequence to be inserted were observed and were confirmed to be approximately parallel, the obtained data were analyzed by the 2^{-ΔΔCt} method, and the results are shown in Table 40. The PCR products were verified to be correct by sequencing.

TABLE 40
Results of primer pair 1 (n = 3, x̅ ± s)

| | | | | ΔΔCt (mean | |
|---|---|---|---|---|---|
| | | | | ΔCt of the | |
| | | Insertion | | experimental | Relative |
| | | of sequence | | group − mean | amount of |
| | | to be | | ΔCt of the | copy number |
| Ct value | GAPDH | inserted | ΔCt | control group 1) | (2^{-ΔΔCt}) |
| Control group 1 | 26.23 | N/A | 13.77 | — | — |
| plate 1 | | | | | |
| Control group 1 | 26.14 | N/A | 13.86 | — | — |
| plate 2 | | | | | |
| Control group 1 | 25.80 | N/A | 14.20 | — | — |
| plate 3 | | | | | |
| Control group 1 | | | 13.94 ± | 0.00 ± | 1.00 |
| mean | | | 0.19 | 0.19 | (0.88-1.14) |
| Experimental | 26.59 | 28.54 | 1.95 | — | — |
| group 1 plate 1 | | | | | |
| Experimental | 26.10 | 27.85 | 1.75 | — | — |
| group 1 plate 2 | | | | | |
| Experimental | 26.29 | 28.41 | 2.12 | — | — |
| group 1 plate 3 | | | | | |
| Experimental | | | 1.94 ± | −12.00 ± | 4096.00 |
| group 1 mean | | | 0.15 | 0.15 | (3691.52-4544.80) |
| Experimental | 26.32 | 30.51 | 4.19 | — | — |
| group 2 plate 1 | | | | | |
| Experimental | 26.42 | 31.03 | 4.61 | — | — |
| group 2 plate 2 | | | | | |
| Experimental | 26.25 | 30.20 | 3.95 | — | — |
| group 2 plate 3 | | | | | |
| Experimental | | | 4.25 ± | −9.69 ± | 826.00 |
| group 2 mean | | | 0.27 | 0.27 | (685.02-996.00) |

From Table 40, it can be seen that compared with the control group, the relative copy number of experiment group 1 was significantly higher than that of the control group, with statistical significance (P<0.05), indicating that the ORF1p and ORF2p expressed by LRE2 in human L1 can also achieve the corresponding gene editing purpose. Meanwhile, compared with the control group, the relative copy number of experiment group 2 was significantly higher than that of the control group, with statistical significance (P<0.05), indicating that the ORF1p and ORF2p expressed in mouse L1 can also achieve the corresponding gene editing purpose. Through this Embodiment, it is demonstrated that ORF1p and/or ORF2p expressed by different L1 species in the human genome, or ORF1p and/or ORF2p expressed by L1 of different species, can be applied to gene editing in the present invention to achieve the corresponding gene editing objectives. At the same time, the ORF1p and ORF2p of human LRE1, the ORF1p and ORF2p of human LRE2, and the ORF1p and ORF2p of mouse also serve as modification sequences for other ORF1p and ORF2p coding sequences to each other. Therefore, this Embodiment also supports the application of modification sequences for ORF1p coding sequences and ORF2p coding sequences.

Embodiment 10 Inspection of the Effect of Gene Editing by Introducing Specific RNA (Partial Alu at 3′ Part) Produced by In Vitro Transcription into a Target System by the Form of RNP with Binding of ORF1p and/or ORF2p Outside the Target System

The Alu used in embodiment 2-9 is Alu Ya5 in the Alu element. In this Embodiment, to test the effects of other types of Alu elements, the sequence of Alu Yb8 was selected for gene editing. The DNA sequence of Alu Yb8 is shown in Seq ID No.51:

GGCCGGGCGCGGTGGCTCACGCCTGTAATCCCAGCACTTTGGGAGGCCG
AGGCGGGTGGATCATGAGGTCAGGAGATCGAGACCATCCTGGCTAACAA
GGTGAAACCCCGTCTCTACTAAAAATACAAAAAAAAAAAATTAGCCGGG
CGCGGTGGCGGGCGCCTGTAGTCCCAGCTACTCGGGAGGCTGAGGCAGG
AGAATGGCGTGAACCCGGGAAGCGGAGCTTGCAGTGAGCCGAGATTGCG
CCACTGCAGTCCGCAGTCCGGCCTAGGCGACAGAGCGAGACTCCGTCTC
AAAAAAAAAAAAAAAAAAAATGTGGCCAAAAGTGTCAGAAA;

Extract partial DNA sequence from Alu Yb8, as shown in Seq ID No.52:

AAAAATACAAAAAAAAAAAATTAGCCGGGCGCGGTGGCGGGCGCCTGTA
GTCCCAGCTACTCGGGAGGCTGAGGCAGGAGAATGGCGTGAACCCGGGA
AGCGGAGCTTGCAGTGAGCCGAGATTGCGCCACTGCAGTCCGCAGTCCG
GCCTAGGCGACAGAGCGAGACTCCGTCTCAAAAAAAAAAAAAAAAAAAA
TGTGGCCAAAAGTGTCAGAAA.

Select a 405 bp sequence from the Lman1 gene in the human genome as shown in Seq ID No.8, insert the sequence to be inserted as shown in Seq ID No.9, and obtain the sequence as shown in Seq ID No.10. Add the T7 promoter sequence as shown in Seq ID No.11 upstream of the sequence shown in Seq ID No.10, and add a partial Alu Yb8 sequence as shown in Seq ID No.52 downstream to obtain the sequence shown in Seq ID No.53:

TAATACGACTCACTATAGGGTAGAGATTCACTGCCTTAGTCTCATGTAGTCT
CGTGTAGTCTTTTGAGTAAATAACATAAAGTATCTCAAGACTTTTTCATAACTTG
ATATTATTTTAGTCTTCCTGAATTTTTAAATATTGAAAAGCTGAGTGTCTTGTCTG
TTTTAGGTGCCTGCACATACTGCATGTGAGAGTCTGGAGACGCCAGACTGTTCTG
AGTCCTGACCTGCTCAGGGGTGAGGTCCCTCTGAGCCTGAGCAAGCATTTCGTAG
CCAACCATGAATTTCCGGACAGTGGCAGAGCGCAGGAGCGGAGGCCTCCCCCTT
ACACTATAGTGACGGGGCTAGTCAAGCTTTGGCAAGTTGCCAGAGGGACTTCCG
CAACAAACCCTATCCTGTCCGAGCAAAGATTACCTATTACCAGAACACACTGAC
AGTAAGTAACATCTATTTAGAGAGAATCAAATAAACAATGTTACAGTATCACTTT
TCATTTTGAATTTTTGATAGAAATTAAATGCACTTAAATTTGGATATGCTTACATA

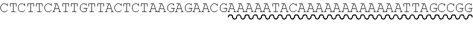

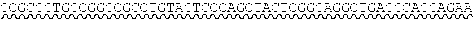

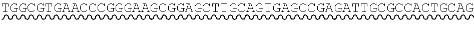

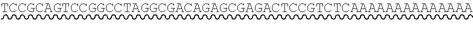

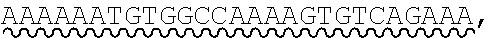

Among them, the underline represents the sequence to be inserted as shown in Seq ID No.9, the italicized bold represents the T7 promoter sequence as shown in Seq ID No.11, and the wavy line represents the partial Alu Yb8 sequence as shown in Seq ID No.52. The sequence was obtained through chemical synthesis and named precursor DNA of RNA+partial Alu Yb8.

According to the instructions of the MEGAscript™ T7 Transcription Kit, the linear precursor DNA of RNA+partial Alu Yb8 was transcribed to obtain corresponding RNA. Then, the residual DNA was degraded by DNase from the kit and resuspended in RNase free water. The RNA concentration was measured by UV spectrophotometer, and the concentration of the solution was further configured to 100 ng/μL by adding RNase free water.

The RNA+partial Alu obtained from the above transcription belongs to the RNA framework structure in FIG. 4, where the ORF2p functional initiating (initiation) part is another type of partial short interspersed element RNA.

Resuspend the hLRE1-ORF1p and hLRE1-ORF2p prepared in Embodiment 1 with Opti-MEM solution pre added with 1 U/μL RNase inhibitor into 500 ng/μL hLRE1-ORF1p solution or hLRE1-ORF2p solution, respectively.

Prepare RNP of RNA+partial Alu Yb8 binding to ORF1p and ORF2p: RNA+partial Alu Yb8+hLRE1-ORF1p+hLRE1-ORF2p, follow the following steps:

Configure the reaction system as shown in Table 41:

TABLE 41
Reaction System

| Component | Addition Amount | |
|---|---|---|
| hLRE1-ORF1p solution | 3.0 | μL |
| RNA + partial Alu Yb8 solution | 21.0 | μL |

| | |
|---|---|
| Opti-MEM ™ I medium | Make up to 50 μL |

Mix the components gently and incubate the reaction system at room temperature (25° C.) for 10 minutes to obtain RNA+partial Alu Yb8+hLRE1-ORF1p solution.

Then, mix the RNA+partial Alu Yb8+hLRE1-ORF1p solution according to the system shown in Table 42.

TABLE 42
Reaction System

| Component | Addition Amount | |
|---|---|---|
| RNA + partial Alu Yb8 + hLRE1-ORF1p solution | 50.0 | μL |
| hLRE1-ORF2p solution | 5.5 | μL |

| | |
|---|---|
| Opti-MEM ™ I medium | Make up to 75 μL |

After gently mixing each component, the reaction system was incubated at room temperature (25° C.) for 10 minutes to obtain RNA+partial Alu Yb8+hLRE1-ORF1p+hLRE1-ORF2p solution.

Mix RNA+partial Alu Yb8+hLRE1-ORF1p+hLRE1-ORF2p solution in equal volume ratio with the transfection solution system prepared in Table 7. Gently mix and incubate at room temperature (25° C.) for 20 minutes to obtain RNA+partial Alu Yb8+hLRE1-ORF1p+hLRE1-ORF2p-liposome complex.

### Transfection

Firstly, Hela cells were passaged and placed on a 24 well plate, cultured in complete medium. The next day after passaging, when Hela cells grew to about 60% confluence, replacing the medium with Opti-MEM™ I medium. According to the instructions of the RNAiMAX transfection reagent, RNA+partial Alu Yb8+hLRE1-ORF1p+hLRE1-ORF2p-liposome complex was added to Hela cells for transfection, with three parallels for each group. Replace the medium with complete medium 6 hours after transfection. Continue to culture cells until they grew to about 90% confluence before passaging. After passaging, repeat transfection once (following the previous steps). After the cells grew again to about 90% confluence, proceeded with subsequent operations.

### Experimental Grouping

There are two groups: the control group in Embodiment 2 was used as the control group in this Embodiment, and the group transfected with RNA+partial Alu Yb8+hLRE1-ORF1p+hLRE1-ORF2p was used as the experimental group. Each group had three parallels, each of which was a 24 well plate cultured with Hela cells.

Extraction of transfected cell DNA in each group: After the medium was aspirated away, the cells were rinsed twice with PBS, digested with an appropriate amount of 0.25% trypsin, and digested at 37° C. for 20 min, with 15 times of pipetting every 5 min. After the cells were suspended, complete medium containing serum was added to stop the reaction (digestion). Thereafter, extraction of cellular DNA was performed according to the product instruction of the blood/cell/tissue genomic DNA extraction kit, and the DNA concentration was determined by an ultraviolet spectrophotometer.

qPCR Detection:

Since the copy number of GAPDH gene is stable, the GAPDH gene was used as a reference gene.

The upstream primer sequence for detecting the GAPDH gene is shown in Seq ID No.19 as follows: 5′-CACTGCCACCCAGAAGACTG-3′. The downstream primer sequence is shown in Seq ID No.20: 5′-CCTGCTTCACCACCTTCTTG-3′.

A primer pair 1 was designed, wherein an upstream primer sequence of the primer pair 1 is shown as Seq ID No.21: 5′-GACTTATCCATGTGCCTGTT-3′; and the downstream primer sequence is shown in Seq ID No.22: 5′-TTGGCTACGAAATGCTTG-3′. The upstream primer sequence of primer pair 1 is located in the complete Lman1 gene, further upstream of the upstream sequence of the insertion site (target site) used on the RNA prepared, not in the RNA prepared, but only in the genome, and the downstream primer sequence of primer pair 1 is located on the exogenous sequence to be inserted (the sequence to be inserted).

The primers were all obtained through chemical synthesis.

The qPCR reaction system is shown in Table 9. Perform qPCR according to the qPCR reaction cycle in embodiment 2. GAPDH primers react under the same conditions.

The exponential growth phases in the amplification curves of GAPDH and the detection of the insertion of the sequence to be inserted were observed and were confirmed to be approximately parallel, the obtained data were analyzed by the 2^{-ΔΔCt} method, and the results are shown in Table 43. The PCR products were verified to be correct by sequencing.

TABLE 43
Results of primer pair 1 (n = 3, x̅ ± s)

| | | | | ΔΔCt (mean | |
|---|---|---|---|---|---|
| | | | | ΔCt of the | |
| | | Insertion | | experimental | Relative |
| | | of sequence | | group − mean | amount of |
| | | to be | | ΔCt of the | copy number |
| Ct value | GAPDH | inserted | ΔCt | control group) | (2^{-ΔΔCt}) |
| Control group | 26.23 | N/A | 13.77 | — | — |
| plate 1 | | | | | |
| Control group | 26.14 | N/A | 13.86 | — | — |
| plate 2 | | | | | |
| Control group | 25.80 | N/A | 14.20 | — | — |
| plate 3 | | | | | |
| Control group | | | 13.94 ± | 0.00 ± | 1.00 |
| mean | | | 0.19 | 0.19 | (0.88-1.14) |
| Experimental | 26.69 | 27.47 | 0.78 | — | — |
| group plate 1 | | | | | |
| Experimental | 26.37 | 27.09 | 0.72 | — | — |
| group plate 2 | | | | | |
| Experimental | 26.24 | 27.22 | 0.98 | — | — |
| group plate 3 | | | | | |
| Experimental | | | 0.83 ± | −13.11 ± | 8841.04 |
| group mean | | | 0.11 | 0.11 | (8192.00-9541.50) |

As shown in Table 43, compared with the control group, the relative amount of the copy number in the experimental group was significantly higher than that in the control group, which was statistically significant (P<0.05), indicating that even if the type of Alu element is changed, the purpose of gene editing can still be achieved. This shows that the present invention can be performed using all types of Alu elements and short interspersed elements in all species.

It can be seen from the above embodiments that the RNA framework provided by the present invention can be expressed in eukaryotic or prokaryotic systems and cells, tissues, organisms or in vitro to produce RNA, and produce the required proteins ORF1p and/or ORF2p in the target system or outside the target system (in vitro), and can be introduced into the target system in the form of RNA or RNP vectors to achieve the goal of gene editing, which is convenient for industrial mass production and commercialization.

In addition, since the splicing mechanism of the precursor mRNA in the eukaryotic system does not exist in the prokaryotic system or in vitro expression, the RNA framework and the downstream connectable short interspersed element RNA, short interspersed element derivative RNA, long interspersed element, long interspersed element derivative RNA, and/or the initiating ORF2p splicing and reverse transcription functional structure can be expressed unimpeded without suffering from potential splicing risks, thereby improving the production efficiency of the present invention and the effect of gene editing.

Therefore, the present invention can perform accurate sequence deletion, sequence replacement (substitution) and replacement (substitution) of individual sites through homologous recombination or genome repair mechanism of the receiving system itself on the basis of targeted insertion of the desired sequence into the genome. At the same time, based on the technical principles of the present invention, it can be known that the present invention can continue to design a vector and insert it through the new site formed after the previous sequence is inserted by the present invention. The progressive insertion makes the length of the inserted sequence theoretically unlimited, and can complete various types and forms of sequence insertion, deletion, replacement and site replacement and other gene editing purposes, and the use method is flexible. In addition, the present invention can also be used to perform gene editing on CNV and its end to stabilize, extend, shorten or change its expression sequence, etc., thereby achieving the purpose of changing or stabilizing the gene expression and self-state of cells or organisms.

Since short interspersed element, long interspersed element and the proteins it expresses are widely present in eukaryotic organisms, the present invention can be used to perform gene editing operations on a wide range of eukaryotic organisms. In addition, it can also be applied to the treatment of diseases with genetic changes and to change or stabilize the state of cells or organisms related to genetic changes, etc. In addition, the present invention can also be used for gene editing of various prokaryotes.

Other gene editing tools such as TALEN, ZFN, Targetron, CRISPR or CRISPR/Cas9 and other technologies introduce sequences into the genome mainly by cutting the genomic target site, and then the template DNA is homologously recombined with the genomic target site and its surrounding area, which is easy to introduce random sequences and mutations, and the efficiency of introducing the target sequence is low. The RNA framework and corresponding RNP provided by the present invention do not cause double-strand breaks, and perform genome integration through homologous recombination, which is relatively safer and convenient for practical application.

The present invention can give the exogenous sequence to the target system in the form of RNA and insert it into the genome, so it can be shown that the RNA is converted into DNA and has the ability to generate template DNA.

Therefore, if the DNA that can express the RNA framework and/or its improved form RNA of the present invention, the RNA framework and/or its improved form RNA of the present invention, and the RNP produced by the RNA framework and/or its improved form RNA combined with ORF2p, ORF1p, ORF2p derived protein and/or ORF1p derived protein of the present invention are given to the target system, the template DNA can be generated without introducing the template DNA, or the template DNA can be generated (amplified) in large quantities. Therefore, the present invention can also improve the gene editing function of other gene editing tools such as TALEN, ZFN, Targetron, CRISPR or CRISPR/Cas9 and other technologies.

In addition, according to the results and principles of the embodiments, the RNA required for gene editing in the present invention can be produced in vitro and combined with ORF2p, ORF1p, ORF2p-derived protein and/or ORF1p-derived protein, and introduced into a prokaryotic system or a eukaryotic system; single-stranded DNA or double-stranded DNA combined with ORF2p, ORF1p, ORF2p-derived protein and/or ORF1p-derived protein is produced and introduced into a target system (such as a prokaryotic system or a eukaryotic system), which can also achieve the purpose of gene editing.

The above-mentioned embodiments only express several implementation methods of the present invention, and the descriptions thereof are relatively specific and detailed, but they cannot be understood as limiting the scope of the patent of the present invention. It should be pointed out that, for ordinary technicians in this field, several variations and improvements can be made without departing from the concept of the present invention, and these all belong to the scope of protection of the present invention. Therefore, the scope of protection of the patent of the present invention shall be subject to the attached claims.

1. An RNA framework for gene editing, comprising an upstream sequence of target site, a sequence to be inserted and a downstream sequence of target site along the direction of 5′→3′;
wherein the upstream sequence of target site on the RNA framework or a complementary sequence of the upstream sequence of target site is used for hybridizing with the upstream sequence of target site or the complementary sequence of the upstream sequence of target site for the target site in an eukaryotic genome or a prokaryotic genome;
the downstream sequence of target site or a complementary sequence of the downstream sequence of target site on the RNA framework is used for hybridizing with the downstream sequence of target site or the complementary sequence of the downstream sequence of target site for the target site in an eukaryotic genome or a prokaryotic genome;
the upstream sequence of target site and the downstream sequence of target site on the RNA framework are directly connected in the corresponding sequence in the genome; and the target site is between the upstream sequence of target site and the downstream sequence of target site in the sequence on the genome.

2. The RNA framework for gene editing according to claim 1, further comprising: one or more ORF2p functional initiation parts are directly or indirectly connected downstream of the downstream sequence of target site, or the downstream sequence of target site of the RNA framework for gene editing is replaced or partially replaced with one or more ORF2p functional initiation parts; wherein the multiple ORF2p functional initiation parts are directly or indirectly connected; wherein the sequence of the ORF2p functional initiation parts is a sequence of a short interspersed element RNA, a long interspersed element RNA, a short interspersed element derivative RNA, a long interspersed element derivative RNA, or a initiating ORF2p splicing and reverse transcription functional structure.

3. The RNA framework for gene editing according to claim 2, wherein one or more pan-ORF1p coding sequences and/or one or more pan-ORF2p coding sequences are further inserted into the ORF2p functional initiation part(s); wherein when one pan-ORF1p coding sequence or one pan-ORF2p coding sequence is inserted into the ORF2p functional initiation part(s), the ORF2p functional initiation part(s) are directly or indirectly connected with the pan-ORF1p coding sequence or the pan-ORF2p coding sequence;
when a) multiple pan-ORF1p coding sequences, or b) multiple pan-ORF2p coding sequences, or c) the sum of the number of the pan-ORF1p coding sequence(s) and the pan-ORF2p coding sequence(s) is greater than or equal to two, pan-ORF1p coding sequence(s) and pan-ORF2p coding sequence(s) are inserted into the ORF2p functional initiation part(s), the pan-ORF1p coding sequences and the pan-ORF2p coding sequences are directly or indirectly connected, the pan-ORF1p coding sequences are directly or indirectly connected, the pan-ORF2p coding sequences are directly or indirectly connected, and the ORF2p functional initiation part(s) are directly or indirectly connected with the pan-ORF1p coding sequence(s) or the pan-ORF2p coding sequence(s),
wherein the sequence of the ORF2p functional initiation parts is a sequence of a short interspersed element RNA, a long interspersed element RNA, a short interspersed element derivative RNA, a long interspersed element derivative RNA, or a initiating ORF2p splicing and reverse transcription functional structure;
wherein the pan-ORF1p coding sequence is a modified sequence of the ORF1p coding sequence or the ORF1p coding sequence, and the pan-ORF2p coding sequence is a modified sequence of the ORF2p coding sequence or the ORF2p coding sequence.

4. The RNA framework for gene editing according to claim 1, further comprising: one or more pan-ORF1p coding sequences and/or one or more pan-ORF2p coding sequences are directly or indirectly connected upstream of the upstream sequence of target site, and/or inside the upstream sequence of target site, and/or inside the downstream sequence of target site, and/or downstream of the downstream sequence of target site,
wherein when the one or more pan-ORF1p coding sequences and/or one or more pan-ORF2p coding sequences are located upstream of the upstream sequence of target site, inside the upstream sequence of target site, inside the downstream sequence of target site, and downstream of the downstream sequence of target site, and when a) the sum of the number of multiple pan-ORF1p coding sequences is greater than or equal to two, or b) the sum of the number of multiple pan-ORF2p coding sequences is greater than or equal to two, or c) the sum of the number of pan-ORF1p coding sequences and pan-ORF2p coding sequences is greater than or equal to two in the same position, the pan-ORF1p coding sequences and the pan-ORF2p coding sequences are directly or indirectly connected, the pan-ORF1p coding sequences are directly or indirectly connected and the pan-ORF2p coding sequences are directly or indirectly connected;
the RNA framework for gene editing further comprising: one or more ORF2p functional initiation parts are directly or indirectly connected downstream of the downstream sequence of target site;
wherein when the one or more pan-ORF1p coding sequences and/or one or more pan-ORF2p coding sequences are located upstream of the upstream sequence of target site, inside the upstream sequence of target site and inside the downstream sequence of target site,
and when a) the sum of the number of multiple pan-ORF1p coding sequences is greater than or equal to two, or b) the sum of the number of multiple pan-ORF2p coding sequences is greater than or equal to two, or c) the sum of the number of pan-ORF1p coding sequences and pan-ORF2p coding sequences is greater than or equal to two in the same position, the pan-ORF1p coding sequences and the pan-ORF2p coding sequences are directly or indirectly connected, the pan-ORF1p coding sequences are directly or indirectly connected and the pan-ORF2p coding sequences are directly or indirectly connected;
wherein when the one or more pan-ORF1p coding sequences and/or one or more pan-ORF2p coding sequences are located downstream of the downstream sequence of target site:
a) when there is one or more ORF2p functional initiation parts and one or more pan-ORF1p coding sequences, the one or more ORF2p functional initiation parts are located before or after the one or more pan-ORF1p coding sequences, or the ORF2p functional initiation parts are spaced apart from the pan-ORF1p coding sequences, the ORF2p functional initiation parts and the pan-ORF1p coding sequences are directly or indirectly connected, the multiple pan-ORF1p coding sequences are directly or indirectly connected, and the multiple ORF2p functional initiation parts are directly or indirectly connected; or
b) when there is one or more ORF2p functional initiation parts and one or more pan-ORF2p coding sequences, the one or more ORF2p functional initiation parts are located before or after the one or more pan-ORF2p coding sequences, or the ORF2p functional initiation parts are spaced apart from the pan-ORF2p coding sequences, the ORF2p functional initiation parts and the pan-ORF2p coding sequences are directly or indirectly connected, the multiple pan-ORF2p coding sequences are directly or indirectly connected, and the multiple ORF2p functional initiation parts are directly or indirectly connected; or
c) when there is one or more ORF2p functional initiation parts, one or more pan-ORF1p coding sequences and one or more pan-ORF2p coding sequences, the ORF2p functional initiation parts are located before or after the one or more pan-ORF1p coding sequences, or before or after the one or more pan-ORF2p coding sequences, or the one or more pan-ORF1p coding sequences are located before or after the one or more pan-ORF2p coding sequences, or the ORF2p functional initiation parts, the pan-ORF1p coding sequences and/or the pan-ORF2p coding sequences are arranged at intervals: the ORF2p functional initiation parts and the pan-ORF1p coding sequences are directly or indirectly connected, the ORF2p functional initiation parts and the pan-ORF2p coding sequences are directly or indirectly connected, and the multiple pan-ORF1p coding sequences are directly or indirectly connected: the multiple pan-ORF2p coding sequences are directly or indirectly connected, the multiple ORF2p functional initiation parts are directly or indirectly connected, and the pan-ORF1p coding sequences and the pan-ORF2p coding sequences are directly or indirectly connected,
wherein when one or more pan-ORF1p coding sequences and/or one or more pan-ORF2p coding sequences are further directly or indirectly connected into a single ORF2p functional initiation part of one or more ORF2p functional initiation parts in the RNA framework, and when one pan-ORF1p coding sequence or one pan-ORF2p coding sequence is inserted into the ORF2p functional initiation parts, the ORF2p functional initiation parts are directly or indirectly connected with the pan-ORF1p coding sequence or the pan-ORF2p coding sequence;
when a) multiple pan-ORF1p coding sequences, or b) multiple pan-ORF2p coding sequences, or c) the pan-ORF1p coding sequence(s) and the pan-ORF2p coding sequence(s), the sum of the number of the pan-ORF1p coding sequences and the pan-ORF2p coding sequences is greater than or equal to two, are inserted into the ORF2p functional initiation parts, the pan-ORF1p coding sequences and the pan-ORF2p coding sequences are directly or indirectly connected, the pan-ORF1p coding sequences are directly or indirectly connected, the pan-ORF2p coding sequences are directly or indirectly connected, and the ORF2p functional initiation parts are directly or indirectly connected with the pan-ORF1p coding sequences or the pan-ORF2p coding sequences;
the RNA framework for gene editing wherein the downstream sequence of target site in the RNA framework is replaced or partially replaced with one or more ORF2p functional initiation parts: wherein when there are multiple ORF2p functional initiation parts, the ORF2p functional initiation parts are directly or indirectly connected;
wherein the sequence of the ORF2p functional initiation parts is a sequence of a short interspersed element RNA, a long interspersed element RNA, a short interspersed element derivative RNA, a long interspersed element derivative RNA, or a initiating ORF2p splicing and reverse transcription functional structure;
wherein the pan-ORF1p coding sequence is a modified sequence of the ORF1p coding sequence or the ORF1p coding sequence, and the pan-ORF2p coding sequence is a modified sequence of the ORF2p coding sequence or the ORF2p coding sequence.

5-33. (canceled)

34. The RNA framework for gene editing according to claim 1, which is obtained by prokaryotic system transcription, eukaryotic system transcription or chemical synthesis.

35. The RNA framework for gene editing according to claim 1, which is a linear RNA or located in a linear RNA, or a circRNA or located in a circRNA;
wherein the linear RNA in which the RNA framework is located or the circRNA in which the RNA framework is located is obtained by prokaryotic system transcription, eukaryotic system transcription or chemical synthesis.

36. The RNA framework for gene editing according to claim 34, wherein the prokaryotic transcription is transcription by an RNA polymerase of a prokaryote; and the eukaryotic transcription is transcription by an RNA polymerase I of an eukaryote, an RNA polymerase II of an eukaryote, or an RNA polymerase III of an eukaryote.

37. An RNP, obtained by binding of the RNA framework for gene editing according to any claim 1 with ORF1p, ORF2p, ORF1p derivative proteins and/or ORF2p derivative proteins.

38. A DNA sequence, transcribing the RNA framework for gene editing according to claim 1.

39. The DNA sequence according to claim 38, wherein a prokaryotic promoter or an eukaryotic promoter is further directly or indirectly connected upstream of, downstream of and/or inside the DNA sequence;
wherein the prokaryotic promoter is T7, T3, T71ac, Sp6, araBAD, trp, lac, Ptac, pL, LacUV5, Tac, pBAD or pR.
wherein the eukaryotic promoter is CMV, pCMV, EF1a, SV40, human PGK1, mouse PGK1, Ubc, human beta actin, CAG, EFT3, TRE, UAS, Ac5, Polyhedrin, CaMKIIa, GAL1, GAL10, GAL1 and GAL10, GAL4, GAL80, TEF1, GDS, ADH1, CaMV35S, Ubi, H1, human U6 or mouse U6 promoter.

40. A DNA vector, having the DNA sequence according to claim 38.

41. A gene editing method, comprising the following steps:
1) selecting the target site to be edited in a genome, and determining an upstream sequence of target site and a downstream sequence of target site on both sides of the target site;
2) preparing the RNA framework for gene editing according to claim 1;
3a) transforming or transfecting the RNA framework into a cell, a tissue, an organ, or an organism to achieve gene editing;
or 3b) transforming or transfecting the linear RNA or the circRNA in which the RNA framework is located into a cell, a tissue, an organ or an organism to achieve gene editing;
or 3c) co-transforming or co-transfecting a variety of the RNA framework, and/or the linear RNA or the circRNA in which the RNA framework is located into a cell, a tissue, an organ or an organism to achieve gene editing;
or 3d) co-transforming or co-transfecting one or more RNA framework, the linear RNA or the circRNA in which the RNA framework is located, and ORF1p, ORF2p, ORF1p derivative proteins and/or ORF2p derivative proteins into a cell, a tissue, an organ or an organism to achieve gene editing.

42. A gene editing method, comprising the following steps:
1) selecting the target site to be edited in a genome, and determining an upstream sequence of target site and a downstream sequence of target site on both sides of the target site;
2) preparing the RNP according to claim 37;
3a) transforming or transfecting the RNP into a cell, a tissue, an organ or an organism to achieve gene editing;
or 3b) co-transforming or co-transfecting one or more RNP, and ORF1p, ORF2p and ORF1p derivative proteins and/or ORF2p derivative proteins into a cell, a tissue, an organ or an organism to achieve gene editing.

43. A gene editing method, comprising the following steps:
1) selecting the target site to be edited in a genome, and determining an upstream sequence of target site and a downstream sequence of target site on both sides of the target site;
2) preparing the DNA sequence according to claim 38; wherein the DNA sequence could be located on a DNA vector and be transcribed by a promoter;
3a) transforming or transfecting the DNA vector into a cell, a tissue, an organ or an organism to achieve gene editing;
or 3b) co-transforming or co-transfecting one or more DNA vector, and ORF1p, ORF2p and ORF1p derivative proteins and/or ORF2p derivative proteins into a cell, a tissue, an organ or an organism to achieve gene editing.

44. A gene editing method, comprising the following steps:
1) selecting the target site to be edited in a genome, and determining an upstream sequence of target site and a downstream sequence of target site on both sides of the target site;
2) preparing the RNA framework for gene editing according to claim 1;
3) preparing one or more auxiliary RNAs comprising ORF2p functional initiation part sequences, one or more pan-ORF1p coding sequences and/or one or more pan-ORF2p coding sequences, and/or one or more auxiliary RNPs obtained by binding the auxiliary RNAs with ORF1p, ORF2p, ORF1p derivative proteins and/or ORF2p derivative proteins, and/or one or more auxiliary DNA vectors that transcribe the ORF2p functional initiation parts, the pan-ORF1p coding sequences and/or the pan-ORF2p coding sequences;
4a) co-transforming or co-transfecting the RNA framework, and the auxiliary RNA, the auxiliary RNP and/or the auxiliary DNA vector prepared in step 3) into a cell, a tissue, an organ or an organism to achieve gene editing;
or 4b) co-transforming or co-transfecting the linear RNA or the circRNA in which the RNA framework for gene editing is located, and the auxiliary RNA, the auxiliary RNP and/or the auxiliary DNA vector prepared in step 3) into a cell, a tissue, an organ or an organism to achieve gene editing;
or 4c) co-transforming or co-transfecting a variety of the RNA framework, the linear RNA or the circRNA in which the RNA framework for gene editing is located, and the auxiliary RNA, the auxiliary RNP and/or the auxiliary DNA vector prepared in step 3) into a cell, a tissue, an organ or an organism to achieve gene editing;
or 4d) co-transforming or co-transfecting one or more the RNA framework, the linear RNA or the circRNA in which the RNA framework for gene editing is located, one or more the auxiliary RNA, the auxiliary RNP and/or the auxiliary DNA vector prepared in step 3), and one or more ORF1p, ORF2p, ORF1p derivative proteins and/or ORF2p derivative proteins into a cell, a tissue, an organ or an organism to achieve gene editing.

45. A gene editing method, comprising the following steps:
1) selecting the target site to be edited in a genome, and determining an upstream sequence of target site and a downstream sequence of target site on both sides of the target site;
2) preparing the RNP according to claim 37;
3) preparing one or more auxiliary RNAs comprising ORF2p functional initiation part sequences, one or more pan-ORF1p coding sequences and/or one or more pan-ORF2p coding sequences, and/or one or more auxiliary RNPs obtained by binding the auxiliary RNAs with ORF1p, ORF2p, ORF1p derivative proteins and/or ORF2p derivative proteins, and/or one or more auxiliary DNA vectors that transcribe the ORF2p functional initiation parts, the pan-ORF1p coding sequences and/or the pan-ORF2p coding sequences;
or 4a) co-transforming or co-transfecting the RNP, and the auxiliary RNA, the auxiliary RNP and/or the auxiliary DNA vector prepared in step 3) into a cell, a tissue, an organ or an organism to achieve gene editing;
or 4b) co-transforming or co-transfecting a variety of the RNP, and the auxiliary RNA, the auxiliary RNP and/or the auxiliary DNA vector prepared in step 3) into a cell, a tissue, an organ or an organism to achieve gene editing;
or 4c) co-transforming or co-transfecting one or more the RNP, one or more the auxiliary RNA, the auxiliary RNP and/or the auxiliary DNA vector prepared in step 3), and one or more ORF1p, ORF2p, ORF1p derivative proteins and/or ORF2p derivative proteins into a cell, a tissue, an organ or an organism to achieve gene editing.

46. A gene editing method, comprising the following steps:
1) selecting the target site to be edited in a genome, and determining an upstream sequence of target site and a downstream sequence of target site on both sides of the target site;
2) preparing the DNA sequence according to claim 38; wherein the DNA sequence could be located on a DNA vector and be transcribed by a promoter.
3) preparing one or more auxiliary RNAs comprising ORF2p functional initiation part sequences, one or more pan-ORF1p coding sequences and/or one or more pan-ORF2p coding sequences, and/or one or more auxiliary RNPs obtained by binding the auxiliary RNAs with ORF1p, ORF2p, ORF1p derivative proteins and/or ORF2p derivative proteins, and/or one or more auxiliary DNA vectors that transcribe the ORF2p functional initiation parts, the pan-ORF1p coding sequences and/or the pan-ORF2p coding sequences;
or 4a) co-transforming or co-transfecting the DNA vector, and the auxiliary RNA, the auxiliary RNP and/or the auxiliary DNA vector prepared in step 3) into a cell, a tissue, an organ or an organism to achieve gene editing;
or 4b) co-transforming or co-transfecting a variety of the DNA vector, and the auxiliary RNA, the auxiliary RNP and/or the auxiliary DNA vector prepared in step 3) into a cell, a tissue, an organ or an organism to achieve gene editing;
or 4c) co-transforming or co-transfecting one or more the DNA vector, one or more the auxiliary RNA, the auxiliary RNP and/or the auxiliary DNA vector prepared in step 3), and one or more ORF1p, ORF2p, ORF1p derivative proteins and/or ORF2p derivative proteins into a cell, a tissue, an organ or an organism to achieve gene editing.

47. The gene editing method according to claim 41, wherein the RNA framework, the linear RNA or the circRNA in which the RNA framework for gene editing is located, transformed, transfected, co-transformed or co-transfected into the cell, the tissue, the organ or the organism is one or more;
when there is one RNA framework or one linear RNA in which the RNA framework for gene editing is located or one circRNA in which the RNA framework for gene editing is located, a single place on the genome is edited;
when the sum of the RNA framework and the linear RNA or the circRNA in which the RNA framework for gene editing is located is greater than or equal to 2, and the upstream sequences of target site and/or the downstream sequences of target site in the RNA framework, the linear RNA or the circRNA in which the RNA framework for gene editing is located are different, multiple places on the genome are edited or operated.

48. An application of the RNA framework for gene editing according to claim 1 or the linear RNA or the circRNA in which the RNA framework for gene editing is located as a drug for preventing and/or treating cancer, a gene-related disease or a neurodegenerative disease;
wherein the cancer is a glioma, a breast cancer, a cervical cancer, a lung cancer, a stomach cancer, a colorectal cancer, a duodenal cancer, a leukemia, a prostate cancer, an endometrial cancer, a thyroid cancer, a lymphoma, a pancreatic cancer, a liver cancer, a melanoma, a skin cancer, a pituitary tumor, a germinoma, a meningioma, a meningeal cancer, a glioblastoma, various astrocytomas, various oligodendrogliomas, an astrodendrocytomas, various ependymomas, a choroid plexus papilloma, a choroid plexus cancer, a chordoma, various gangliocytomas, a olfactory neuroblastoma, a sympathetic nervous system neuroblastoma, a pinealocytoma, a pineal blastoma, a medulloblastoma, a retina blastoma, a trigeminal schwannoma, a facial acoustic neuroma, a Glomus jugulare tumor, an angioreticuloma, a craniopharyngioma or a granular cell tumor;
wherein the gene-related disease is Huntington's disease, fragile X syndrome, phenylketonuria, pseudohypertrophic progressive muscular dystrophy, Duchenne muscular dystrophy, mitochondrial encephalomyopathy, mucopolysaccharidosis type I, mucopolysaccharidosis type II, mucopolysaccharidosis type IIIA, mucopolysaccharidosis type IIIB, mucopolysaccharidosis type IIIC, mucopolysaccharidosis type IIID, mucopolysaccharidosis type IVA, mucopolysaccharidosis type IVB, mucopolysaccharidosis type VI, mucopolysaccharidosis type VII, mucopolysaccharidosis type IX, spinal muscular atrophy, Parkinson's disease Syndrome, albinism, red green blindness, chondrodysplasia, enuresis, congenital deafness, thalassemia, sickle cell anemia, hemophilia, epilepsy related to genetic changes, myoclonus, dystonia, stroke and schizophrenia, vitamin D resistant rickets Familial colonic polyposis, 21 hydroxylase deficiency, arginase deficiency, Alport syndrome, Angelman syndrome, Reyna syndrome, atypical hemolytic uremia, autoimmune encephalitis, autoimmune pituitary inflammation, autoimmune insulin receptor disease, β-ketolytic enzyme deficiency, biotinidase deficiency, cardiac ion channel disease, primary carnitine deficiency, Castleman's disease, Charcot Marie Tooth disease, citrullinemia, congenital adrenal dysplasia, congenital hyperinsulinemia, congenital myasthenia gravis syndrome, non nutritive muscular rigidity syndrome, congenital scoliosis, coronary artery ectasia, congenital pure red blood cell aplasia Anemia Erdheim Chester's disease, Fabre's disease, familial Mediterranean fever, Fanconi anemia, galactosemia, Gaucher's disease, systemic myasthenia gravis, Gitelman syndrome, glutaratemia type I, glycogen storage disease (type I, type II), hemophilia, Wilson's disease, hereditary angioedema, hereditary epidermolysis bullosa, hereditary fructose intolerance, hereditary hypomagnesemia, hereditary multiple cerebral infarction dementia, hereditary spastic paraplegia, holocarboxylase synthase deficiency, homocysteinemia, homozygous familial hypercholesterolemia, HHH syndrome, hyperphenylalaninemia, hypoalkaline phosphatasia, hypophosphatemic rickets Diseases, idiopathic cardiomyopathy, idiopathic hypogonadotropic hypogonadism, idiopathic pulmonary hypertension, idiopathic pulmonary fibrosis IgG4 related diseases, congenital bile acid synthesis disorder, isovaleric acidemia, Kalman syndrome, Langerhans histiocytosis, Leren's syndrome, Leber hereditary optic neuropathy, long-chain 3-hydroxyacyl coenzyme A dehydrogenase deficiency, lymphangiomyomatosis, lysinuria protein intolerance, lysosomal acid lipase deficiency, maple syrup urine syndrome, Marfan syndrome, McCune Albright syndrome, medium chain acyl coenzyme A dehydrogenase deficiency, methylmalonic acidemia, multifocal motor neuropathy, multifocal acyl coenzyme A dehydrogenase deficiency, multiple sclerosis, Ankylosing muscular dystrophy, N-acetylglutamate synthase deficiency, neonatal diabetes, neuromyelitis optica, Niemann Pick disease, nonsyndromic deafness Noonan syndrome, ornithine aminotransferase deficiency, osteogenesis imperfecta, juvenile Parkinson's disease, early-onset Parkinson's disease, paroxysmal nocturnal hemoglobinuria, black spot polyp syndrome, POEMS syndrome, porphyria, Prader Willi syndrome, primary combined immunodeficiency, primary hereditary dystonia, primary light chain amyloidosis, progressive familial intrahepatic cholestasis, progressive muscular dystrophy, propionemia, alveolar proteinosis, pulmonary cystic fibrosis, retinitis pigmentosa, severe congenital neutropenia, severe infantile myoclonic epilepsy, Dravet syndrome Silver Russell syndrome, sitosterolemia, spinal medullary muscular atrophy, spinal muscular atrophy, spinocerebellar ataxia, systemic sclerosis, tetrahydrobiopterin deficiency, tuberous sclerosis, primary tyrosinemia, very long-chain acyl CoA dehydrogenase deficiency, Williams syndrome, eczema thrombocytopenia with immunodeficiency syndrome, X-linked agammaglobulinemia, X-linked adrenal leukodystrophy, X-linked lymphoproliferative disorder, arteriosclerotic cerebral small vessel disease, cerebral amyloid angiopathy, common cerebral artery disease with subcortical infarction and white matter encephalopathy, hidden cerebral artery disease with subcortical infarction and white matter encephalopathy, Cathepsin A-related arterial disease in stroke and white matter encephalopathy, pyridoxine dependent epilepsy, AADC enzyme deficiency in serotonin metabolism AADC deficiency or hereditary nephritis;
wherein the neurodegenerative disease is Parkinson's disease, Alzheimer's disease, Huntington's disease, amyotrophic lateral sclerosis, spinocerebellar ataxia, multiple system atrophy, primary lateral sclerosis, Pick's disease, frontotemporal dementia, Lewy body dementia, or progressive supranuclear palsy.

49. An application of the RNA framework for gene editing according to claim 1 or the linear RNA or the circRNA in which the RNA framework for gene editing is located as a tool for insertion of target sequences, deletion of target sequences, replacement of target sequences, deletion of target sites, addition of target sites, addition of target sequences, replacement of target sites, inversion of target gene sequences, and/or inversion correction of target gene sequences.

50. An application of the RNP according to claim 37 as a tool for insertion of target sequences, deletion of target sequences, replacement of target sequences, deletion of target sites, addition of target sites, addition of target sequences, replacement of target sites, inversion of target gene sequences, and/or inversion correction of target gene sequences.

51. An application of the DNA sequence according to claim 38 or the DNA vector transcribing the DNA sequence on it as a tool for insertion of target sequences, deletion of target sequences, replacement of target sequences, deletion of target sites, addition of target sites, addition of target sequences, replacement of target sites, inversion of target gene sequences, and/or inversion correction of target gene sequences.

52. An application of the RNA framework for gene editing according to claim 1 or the linear RNA or the circRNA in which the RNA framework for gene editing is located in production or amplification of a DNA template comprising the sequence of the RNA framework.

53. An application of the RNP according to claim 37 in production or amplification of a DNA template comprising the sequence of the RNA framework.

54. An application of the DNA vector according to claim 38 in production or amplification of a DNA template comprising the sequence of the RNA framework.

55. An application of the RNA framework for gene editing according to claim 1 or the linear RNA or the circRNA in which the RNA framework for gene editing is located as a tool for increasing the gene editing efficiency of TALEN, ZFN, Targetron, Prime Editor, Twin Prime Editor, CRISPR or CRISPR/Cas9 technologies.

56. An application of the RNP according to claim 37 as a tool for increasing the gene editing efficiency of TALEN, ZFN, Targetron, Prime Editor, Twin Prime Editor, CRISPR or CRISPR/Cas9 technologies.

57. An application of the DNA vector according to claim 38 as a tool for increasing the gene editing efficiency of TALEN, ZFN, Targetron, Prime Editor, Twin Prime Editor, CRISPR or CRISPR/Cas9 technologies.

58. An application of the DNA template according to claim 52 as a tool for increasing the gene editing efficiency of TALEN, ZFN, Targetron, Prime Editor, Twin Prime Editor, CRISPR or CRISPR/Cas9 technologies.

59. An application of the DNA template according to claim 53 as a tool for increasing the gene editing efficiency of TALEN, ZFN, Targetron, Prime Editor, Twin Prime Editor, CRISPR or CRISPR/Cas9 technologies.

60. An application of the DNA template according to claim 54 as a tool for increasing the gene editing efficiency of TALEN, ZFN, Targetron, Prime Editor, Twin Prime Editor, CRISPR or CRISPR/Cas9 technologies.
